(19) United States
(12) Patent Application Publication
(10) Pub. No.: US 2007/0142345 A1
(43) Pub. Date: Jun. 21, 2007

(54) Steroids for cancer treatment

(76) Inventor: Lars Pettersson, Sodra Sandby (SE)

Correspondence Address: FISH & RICHARDSON P.C., PO BOX 1022, MINNEAPOLIS, MN, 55440-1022, US

(21) Appl. No.: 10/587,561

(22) PCT Filed: Feb. 11, 2005
(86) PCT No.: PCT/SE05/00188

§ 371 Date: Jul. 31, 2006

Related U.S. Application Data
(60) Provisional application No. 60/543,922, filed on Feb. 13, 2004.

(30) Foreign Application Priority Data
Feb. 13, 2004 (SE) 0400346-3

(51) Int. Cl. A61K 31/56; C07J 53/00

(52) U.S. Cl. 514/182; 552/508

(57) ABSTRACT

The present invention relates to novel compounds which are 7α-substituted 17-alkylene-16α-hydroxy steroidal estrogens. This invention specifically relates to estrogen derivatives which contain 7α-substituents and which exhibit anti-estrogenic properties. The present invention also relates to use of said compounds as a medicament, and for the treatment of estrogen dependent disorders, a pharmaceutical composition comprising one or more of said compounds and a method of treatment.

### FIELD OF THE INVENTION

The present invention relates to novel compounds which are 7α-substituted 17-alkylene-16αhydroxy steroidal estrogens. This invention specifically relates to estrogen derivatives which contain a non-standard D-ring substitution pattern and which exhibit anti-estrogenic properties. The present invention also relates to use of said compounds as a medicament, and for the treatment of estrogen dependent disorders, a pharmaceutical composition comprising one or more of said compounds and a method of treatment.

### BACKGROUND

Estrogens are small molecule ligands that bind to the ligand-binding domain (LBD) of the estrogen receptors ER-α and ER-β. The ligand-receptor complex regulates the transcription of certain genes by binding to response elements in the promotor regions of the genes. The receptor protein activates the transcription machinery by a complex mechanism, through the activating functions AF-1 and AF-2 in the ER. For a comprehensive review on (anti)-estrogens, their receptors, structure and function, see ref 1.

There are broadly speaking three types of ligands, all binding to the LBD but showing different pharmacological profiles: the full agonists, e.g. estradiol, which activate through both the AF-1 and the AF-2 activating functions of the receptor; the mixed agonists/antagonists or the so called SERMs (selective ER modulators), e.g. raloxifen, which activate only through the AF-1 and behave either as agonists or as antagonists depending on the cellular context and tissue; the full antagonists, e.g. ICI 182,780, which inhibit both the AF-1 and the AF-2 activating functions.

The full antagonists, the so-called pure anti-estrogens, were first described by Bowler et al. (ref 2) and are especially useful for the treatment of breast cancer.

The molecular mechanisms at the level of ligand-receptor complex differentiating the full agonist, the SERM, and the full antagonist have recently been elucidated by X-ray crystallography (ref 3,4).

It has been speculated that the 11- and 7(-substituents, both for antagonists and agonists, may bind to a common pocket in the receptor protein (ref 5).

Recently it was shown that the full antagonist ICI 164,384 binds to the LBD of ERP in a 180° flipped orientation around the 03-017 axis, compared with the estra-diol-ER complex (ref 4). In this orientation the 7α-substituent of ICI 164,384 can occupy the so-called 11β-pocket of the receptor LBD.

In order to show potent agonistic effects steroidal estrogens should have a 17-hydroxy group, preferably a 17β-hydroxy, or a 17-keto group. The 17β-hydroxy group in such compounds is often combined with e.g. 17α-alkyl (or -alkynyl) or 16α-halide substituents. This type of D-ring substitution pattern has also been used in the 11β- or 7α-substituted steroidal anti-estrogens reported in the literature, including 7α-substituted steroidal compounds.

In EP0138504 17β-hydroxy substituted, optionally derivatized, or 17-keto substituted steroidal compounds are reported. This document includes the compound ICI 182,780 (3,17β-dihydroxy -7α-(9-[[(4,4,5,5,5-pentafluoro-n-pentyl)sulfinyl]nonyl]-estra-1,3,5(10)-triene))

EP0280618 describes 7α-aryl substituted steroids, including anti-estrogens, which all are 17β-hydroxy, 17β-acyloxy, or 17β-alkoxy substituted compounds.

EP0367576 discloses compounds for use in the inhibition of sex steroid activity. Among these compounds are 7α-substituted estratrienes, preferably substituted with a 17-hydroxy or a 17-keto group.

WO9920646 reports steroidal estrogens and anti-estrogens. The compounds are 17-hydroxy, 17-acyloxy, 17-alkoxy, or 17-keto substituted in the D-ring. The 17β-derivatives are preferred.

In WO0142186 compounds having hydroxycarbonyl-haloge-noalkyl side chains are reported. Some of these compounds are described as 7w-substituted steroidal anti-estrogens, all of which have the 17β-hydroxy substitution pattern.

In EP0410554 7α-substituted 14,17α-ethano- and -ethenoestratrienes are reported as anti-estrogenic compounds. The compounds are all 17β-hydroxy derivatives.

EP0906332 (DE 19622457) reports on 7α-(5-methyl-aminopentyl)-estratrienes and W09933855 reports on 11β-halogen-7α-substituted estrogens. All compounds are 17β-hydroxy or 17β-acyloxy derivatives.

In WO9807740 7α-aminoalkyl-estratrienes are described, all compounds being 17-hydroxy or -acyloxy derivatives. The vast majority of cited compounds are 17β-hydroxy derivatives.

### SUMMARY OF THE INVENTION

As can be seen, known anti-estrogenic compounds contain a hydroxy group or a hydroxy derivative at the 17-position, particularly a 17β-hydroxy. This is considered to be essential to obtain high binding affinity. Indeed, replacing the 17β-hydroxy substitution pattern of a regular steroidal estrogen with a 17-alkylene-16-α-hydroxyl substitution leads to steroidal estrogens with low “sex hormonal” activities, as has been described in WO9708188. This indicates a low binding affinity and/or low estrogenic agonistic potency of compounds with this D-ring substitution pattern.

The objective problem of the present invention is to develop novel steroidal anti-estrogen compounds with a new D-ring substitution pattern, that does not include the above mentioned substitution pattern known for potent estrogens, but still with a retained or higher affinity for the estrogen receptor in comparison with the above disclosed compounds.

Novel compounds with these properties take the form of new high affinity steroidal anti-estrogens according to formula I. These contain a 17-alkylene-16α-hydroxyl substitution pattern in the D-ring in combination with a side-chain at the 7α-position.

The inventor of the present invention have unexpectedly found that the compounds of the present invention show higher affinity to the ERα-receptor, compared with known anti-estrogens. In other words—contrary to expectations—the affinity of the compounds has not been lost when altering the substitution pattern of the D-ring. Particularly of interest are those compounds showing activity which is suprisingly higher than those of the prior art.

Compounds of the present invention that show pure antiestrogenic activity are especially useful for the treatment of estrogen dependent breast cancer and other estrogen related disorders such as anovulatory infertility, menstrual disorders, male pattern baldness, dysfunctional uterine bleeding, endometrial polyps, benign breast disease, uterine leiomyomas, adenomyosis, ovarian cancer, endometrial cancer, melanoma, prostate cancer, cancers of the colon, CNS cancers, endometriosis, polycystic ovary syndrome, infertility, and can also be used for contraception in males.

The phrases “antagonistic properties” and “antiestrogenic properties” used in the present application relates to compounds that antagonise the action of an estrogen at the receptor level.

### DETAILED DESCRIPTION OF THE INVENTION

The object of the present invention is to provide novel compounds which are 7α-substituted 17-alkylene-16α-hydroxy steroidal estrogens.

In a first aspect the present invention relates to an anti-estrogenic compound of the general formula I 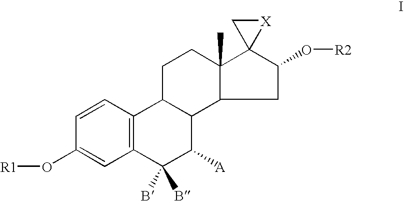
wherein
A is a 8-22 atoms long substituent, which substituent A is defined by D₁₋₆, wherein D is chosen from the group comprising R4-C(O)R4, R4S(O)₀₋₂R4, N(R4)₃, R4OR4 and R4(C6H4)R4
wherein R4 independently represents a bond, or H, or a halogenated or non-halogenated, saturated or unsaturated, mono-, di-, or trivalent C1-C12 hydrocarbon
B′,B″ are H,H or H,O—R3 or O—R3,H or H,F or together represent ═O;
R1 is H, or a potentially metabolically unstable group chosen from the group comprising a straight, branched, or cyclic C1-C6 alkyl, C1-C6 acyl, benzoyl, sulphamoyl, or N-acetyl-sulphamoyl;
R2 is H, or a potentially metabolically unstable group chosen from the group comprising C1-C6 acyl or benzoyl;
R3 is H, or C1-C3 alkyl, or a metabolically unstable group chosen from the group comprising C1-C6 acyl, benzoyl, sulphamoyl, or N-acetyl-sulphamoyl; and
X is methylene or a single bond, or pharmaceutically acceptable salts of the compounds of the general formula I.

In embodiments of the present invention, A is
—(CH₂)₄₋₆N((CH₂)₀₋₂H)(CH₂)₂₋₄S(O)₀₋₂(CH₂)₂₋₄(CF₂)₁₋₃CF₃
   or A is
—(CH₂)₇₋₁₁S(O)₀₋₂(CH₂)₂₋4(CF₂)₁₋₃CF₃
   or A is
—(CH₂)₈₋₁₂C(O)N((CH₂)₀₋₂H)(CY₂)₂₋₆Y

wherein Y is chosen from H or F.

In a further embodiment, R1 is hydrogen, or methyl, or acetyl, or benzoyl, or sulphamoyl, or N-acetyl-sulphamoyl.

Furthermore, R3 may be H, or methyl, or a potentially metabolically unstable group chosen from the group comprising C1-C6 acyl, benzoyl, sulphamoyl, or N-acetyl-sulphamoyl.

In one preferred embodiment of the present invention, A is
—(CH₂)₄₋₆N((CH₂)₀₋₂H)(CH₂)₂₋₄S(O)₀₋₂(CH₂)₂₋₄(CF₂)₁₋₃CF₃
   or
—(CH₂)₇₋₁₁S(O)₀₋₂(CH₂)₂₋₄(CF₂)₁₋₃CF₃
   or
—(CH₂)₈₋₁₂C(O)N((CH₂)₀₋₂H)(CY₂)₂₋₆Y

wherein Y is chosen from H or F

or

—(CH₂)₈₋₉CH(CO₂H)(CH₂)₂₋₅(CF₂)₁₋₃CF₃
   or
—C₆H₄-p-O(CH₂)₃₋₆S(O)₀₋₂(CH₂)₂₋₄(CF₂)₁₋₃CF₃
   or
—C₆H₄-p-O(CH₂)₂NMe₂;
   R1 is hydrogen, or methyl, or acetyl, or benzoyl, or sulphamoyl, or N-acetyl-sulphamoyl;
   R2 is hydrogen; and
   R3 is H, or methyl, or a potentially metabolically unstable group chosen from the group comprising C1-C6 acyl, benzoyl, sulphamoyl, or N-acetyl-sulphamoyl.

In another preferred embodiment A is
—(CH₂)₄₋₆N(CH₃)(CH₂)₂₋₄S(O)₀₋₂(CH₂)₂₋₄(CF₂)₁₋₃CF₃
   or
—(CH₂)₇₋₁₁S(O)₀₋₂(CH₂)₂₋₄(CF₂)₁₋₃CF₃
   or
—(CH₂)₁₀C(O)N(CH₃)(CY₂)₂₋₆Y

wherein Y is chosen from H or F

or

—(CH₂)₈₋₉CH(CO₂H)(CH₂)₂₋₅(CF₂)₁₋₃CF₃;
   B′,B″ are H,H or H,O—R3 or O—R3,H or H,F;
   R1 is H, or methyl, or acetyl, or sulphamoyl; and
   R3 is H, or methyl, or acyl;
   In still another preferred embodiment of the present invention A is
—(CH₂)₄₋₆N(CH₃)(CH₂)₃S(O)₀₋₂(CH₂)₃CF₂CF₃
   or
—(CH₂)₈₋₁₀S(O)₀₋₂(CH₂)₂₋₄(CF₂)₁₋₃CF₃
   or
—(CH₂)₈₋₉CH(CO₂H)(CH₂)₂₋₅(CF₂)₁₋₃CF₃
   and
   R3 is H.

In yet another embodiment the new compound discribed above is chosen from the group comprising
11-(3,16α-Dihydroxy-17-methylene-estra-1,3,5(10)-triene-7α-yl)-undecanoic acid n-butyl-methyl-amide, 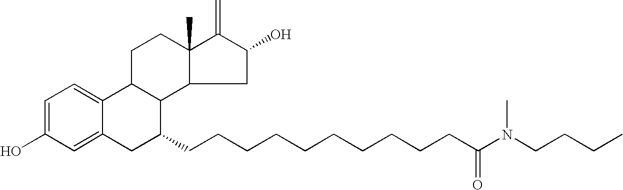
11-(3,16α-Dihydroxy-17-methylene-estra-1,3,5(10)-triene-7α-yl)-undecanoic acid n-butyl-methyl-amide 3-O-benzoate, 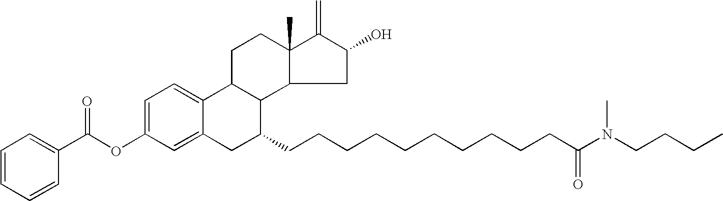
11-(3,16α-Dihydroxy-17-methylene-estra-1,3,5(10)-triene-7α-yl)-undecanoic acid (2,2,3,3,4,4,4-heptafluoro)-n-butyl-methyl-amide, 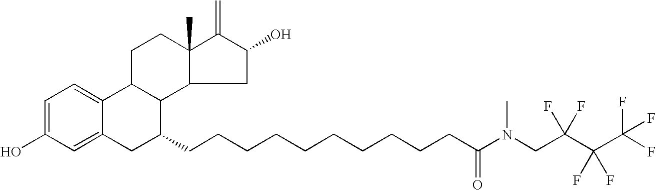
3,16α-Dihydroxy-17-methylene-7α-[9-[(4,4,5,5,5-penta-fluoro-n-pentyl)thio]nonyl]-estra-1,3,5(10)-triene, 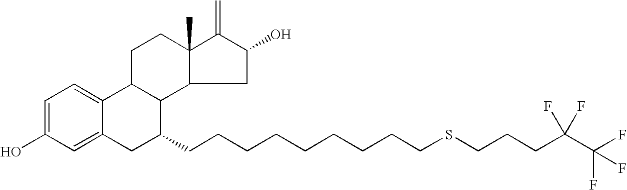
3,16α-Dihydroxy-17-methylene-7α-[9-[(4,4,5,5,5-penta-fluoro-n-pentyl)sulfinyl]nonyl]-estra-1,3,5(10)-triene, 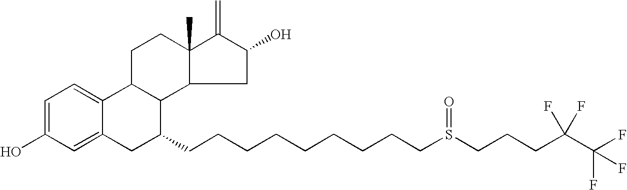
3,16α-Dihydroxy-17-methylene-7α-[9-[(4,4,5,5,5-penta-fluoro-n-pentyl)sulfinyl]nonyl]-estra-1,3,5(10)-triene 3-)O-acetate, 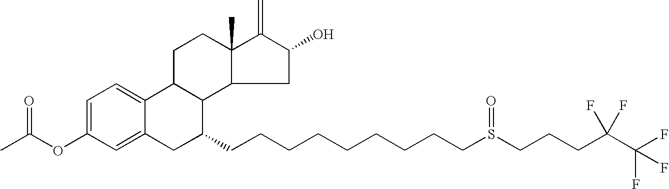
3,16α-Dihydroxy-17-methylene-7α-[9-[(4,4,5,5,5-penta-fluoro-n-pentyl)sulfinyl]nonyl]-estra-1,3,5(10)-triene 3-O-sulfamate, 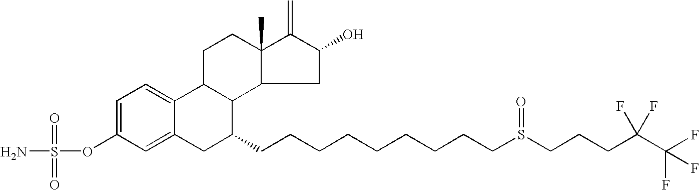
3,16α-Dihydroxy-17-methylene-7α-[9-[(4,4,5,5,5-penta-fluoro-n-pentyl)sulfinyl]nonyl]-estra-1,3,5(10)-triene 3-O-benzoate, 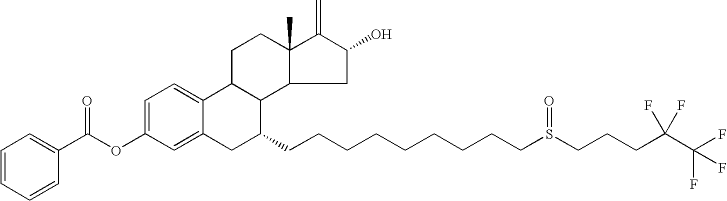
3,16α-Dihydroxy-17-methylene-7α-[9-[(4,4,5,5,5-penta-fluoro-n-pentyl)sulfonyl]nonyl]-estra-1,3,5(10)-triene, 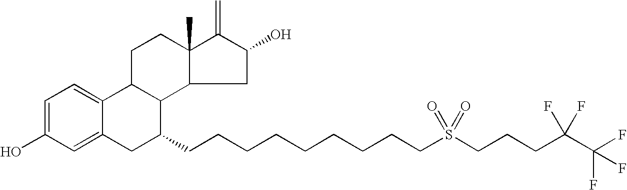
3,16α-Dihydroxy-17-methylene-7α-[9-[(4,4,5,5,5-penta-fluoro-n-pentyl)sulfinyl]octyl]-estra-1,3,5(10)-triene, 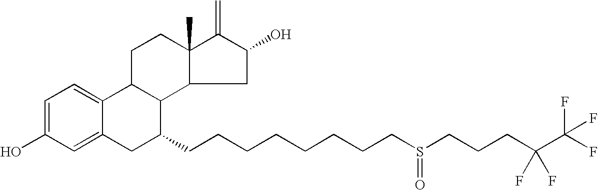
7α-[9-[(2,2,3,3,4,4,4-Heptafluoro-n-butyl)sulfinyl]nonyl]-3,16α-dihydroxy-17-methylene-estra-1,3,5(10)-triene, 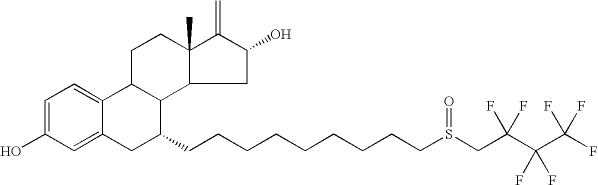
3,16α-Dihydroxy-17-methylene-7α-[9-[(3,3,4,4,5,5,6,6,6-nonafluoro-n-hexyl)sulfonyl]nonyl]-estra-1,3,5(10)-triene, 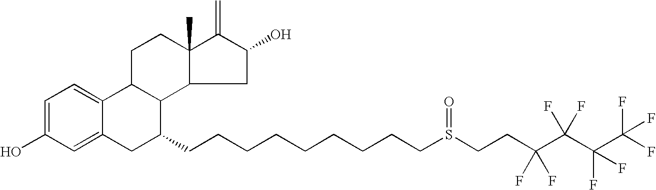
3,16α-Dihydroxy-17-methylene-7α-[9-[(4,4,5,5,6,6,7,7,7-nonafluoro-n-heptyl)sulfonyl]nonyl]-estra-1,3,5(10)-triene, 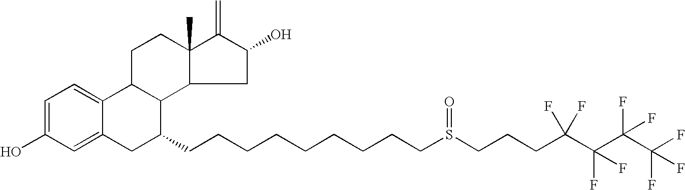
3,16α-Dihydroxy-17-methylene-7α-[5-[N-methyl-N-3-(4,4,5,5,5-pentafluoro-n-pentylthio)-propylamino]-pentyl]-estra-1,3,5(10)-triene, 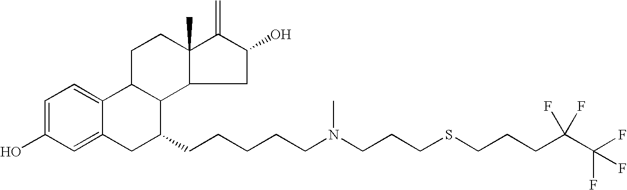
3,16α-Dihydroxy-17-methylene-7α-[5-[N-methyl-N-3-(4,4,5,5,5-pentafluoro-n-pentylsulfinyl)-propylamino]-pentyl]-estra-1,3,5(10)-triene, 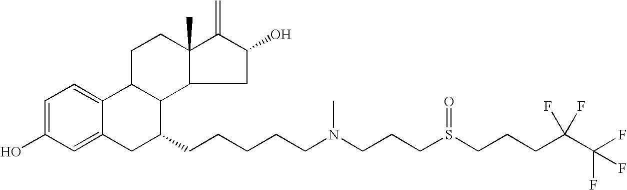
3,16α-Dihydroxy-17-methylene-7α-[5-[N-methyl-N-3-(4,4,5,5,5-pentafluoro-n-pentylsulfinyl)-propylamino]-pentyl]-estra-1,3,5(10)-triene 3-O-sulfamate, 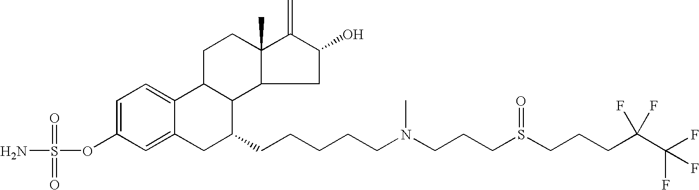
3,16α-Dihydroxy-17-methylene-7α-[5-[N-methyl-N-3-(4,4,5,5,5-pentafluoro-n-pentylsulfinyl)-propylamino]-pentyl]-estra-1,3,5 (10)-triene 3-O-benzoate, 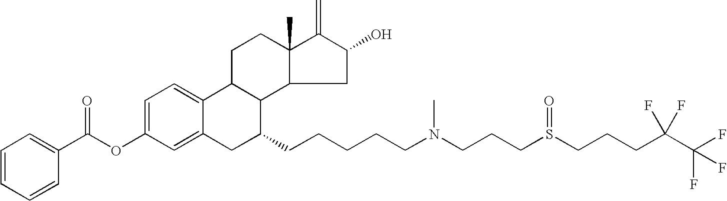
3,16α-Dihydroxy-17-methylene-7α-[5-[N-methyl-N-3-(4,4,5,5,5-pentafluoro-n-pentylsulfonyl)-propylamino]-pentyl]-estra-1,3,5(10)-triene, 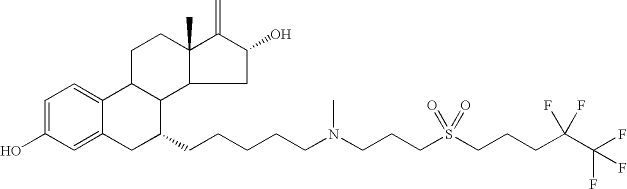
3,16α-Dihydroxy-17-methylene-7α-[5-[N-methyl-N-3-(3,3,4,4,5,5,6,6,6-nonafluoro-n-hexyl)-propylamino]-pentyl]-estra-1,3,5(10)-triene, 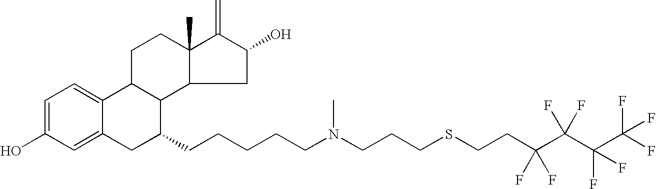
3,16α-Dihydroxy-17-methylene-7α-[5-[N-methyl-N-3-(4,4,5,5,6,6,7,7,7-nonafluoro-n-heptyl)-propylamino]-pentyl]-estra-1,3,5(10)-triene, 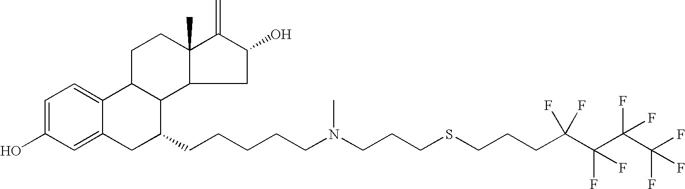
11-(3,16α-Dihydroxy-17-methylene-estra-1,3,5(10)-triene-7α-yl)-2-(4,4,5,5,5-pentafluoro-n-pentyl)-undecanoic acid, 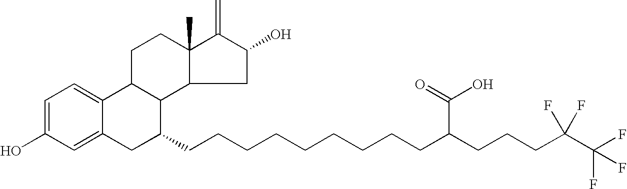
11-(3,16α-Dihydroxy-17-methylene-estra-1,3,5(10)-triene-7α-yl)-2-(4,4,5,5,6,6,7,7,7-nonafluoro-n-heptyl)-undecanoic acid, 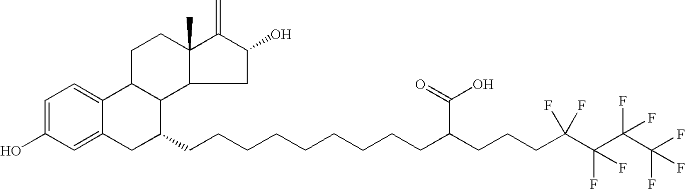
11-(3,16α-Dihydroxy-17-methylene-estra-1,3,5(10)-triene-7α-yl)-2-(3,3,4,4,5,5,6,6,6-nonafluoro-n-hexyl)-undecanoic acid, 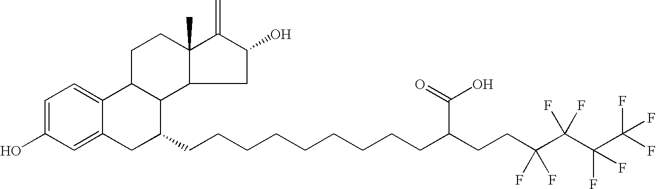
10-(3,16α-Dihydroxy-17-methylene-estra-1,3,5(10)-triene-7α-yl)-2-(3,3,4,4,5,5,6,6,6-nonafluoro-n-hexyl)-decanoic acid, 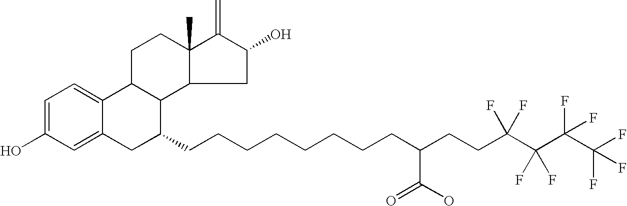
11-(3,16α-Dihydroxy-17-methylene-estra-1,3,5(10)-triene-7α-yl)-2-(3,3,4,4,5,5,6,6,6-nonafluoro-n-hexyl)-undecanoic acid methylester, 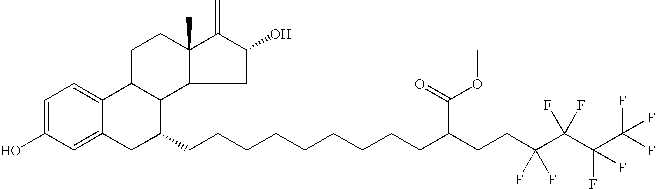
2-[9-(3,16α-Dihydroxy-17-methylene-estra-1,3,5(10)-triene-7α-yl)-nonyl]-2-(3,3,4,4,5,5,6,6,6-nonafluoro-n-hexyl)-malonic acid, 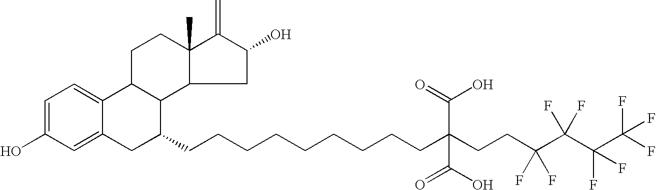
11-(3,6α,16α-Trihydroxy-17-methylene-estra-1,3,5(10)-triene-7α-yl)-undecanoic acid n-butyl-methyl-amide, 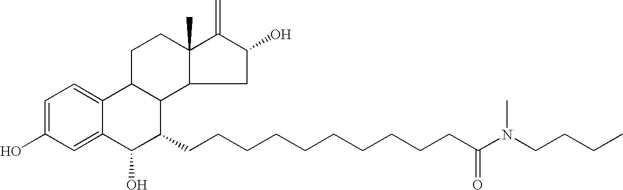
3,6α,16α-Trihydroxy-17-methylene-7α-[9-[(4,4,5,5,5-pentafluoro-n-pentyl)thio]nonyl]-estra-1,3,5(10)-triene, 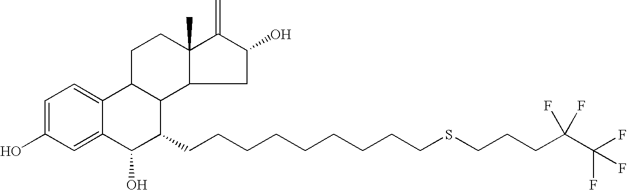
3,6α,16α-Trihydroxy-17-methylene-7α-[9-[(4,4,5,5,5-pentafluoro-n-pentyl)sulfinyl]nonyl]-estra-1,3,5 (10)-triene, 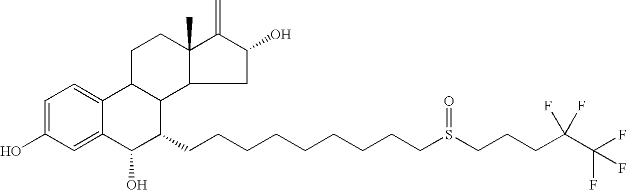
3,6α,16α-Trihydroxy-17-methylene-7α-[9-[(4,4,5,5,5-pentafluoro-n-pentyl)sulfinyl]nonyl]-estra-1,3,5(10)-triene 3-O-sulfamate, 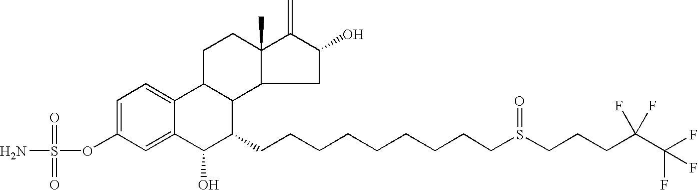
3,6α,16α-Trihydroxy-17-methylene-7α-[5-[N-methyl-N-3-(4,4,5,5,5-pentafluoro-n-pentylthio)-propylamino]-pentyl]-estra-1,3,5(10)-triene, 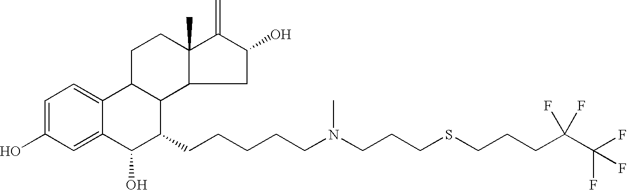
3,6α,16α-Trihydroxy-17-methylene-7α-[5-[N-methyl-N-3-(4,4,5,5,5-pentafluoro-n-pentylthio)-propylamino]-pentyl]-estra-1,3,5(10)-triene 3-O-sulfamate, 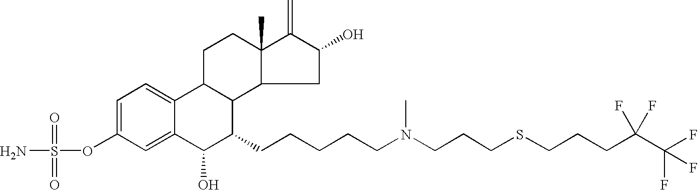
3,6α,16α-Trihydroxy-17-methylene-7α-[5-[N-methyl-N-3-(4,4,5,5,5-pentafluoro-n-pentylsulfinyl)-propylamino]-pentyl]-estra-1,3,5(10)-triene, 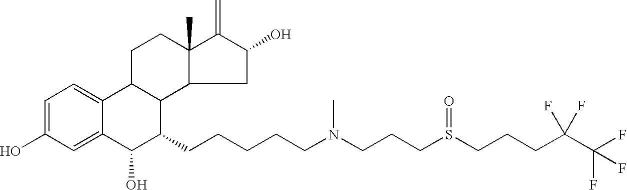
3,6α,16α-Trihydroxy-17-methylene-7α-[5-[N-methyl-N-3-(3,3,4,4,5,5,6,6,6-nonafluoro-n-hexyl)-propylamino]-pentyl]-estra-1,3,5(10)-triene, 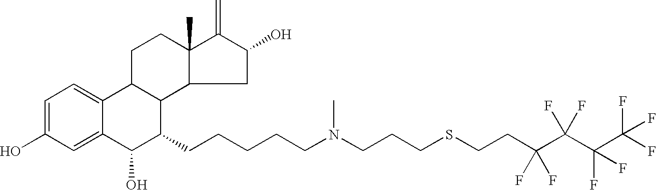
11-(3,6α,16α-Trihydroxy-17-methylene-estra-1,3,5(10)-triene-7α-yl)-2-(3,3,4,4,5,5,6,6,6-nonafluoro-n-hexyl)-undecanoic acid, 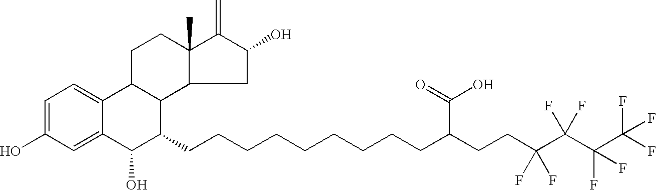
10-(3,6α,16α-Trihydroxy-17-methylene-estra-1,3,5(10)-triene-7α-yl)-2-(3,3,4,4,5,5,6,6,6-nonafluoro-n-hexyl)-decanoic acid, 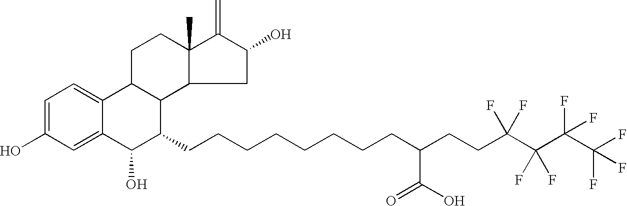
11-(6β-Fluoro-3,16α-dihydroxy-17-methylene-estra-1,3,5(10)-triene-7α-yl)-undecanoic acid n-butyl-methyl-amide, 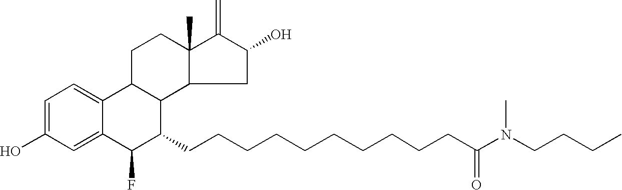
16β-Fluoro-3,16α-dihydroxy-17-methylene-7α-[9-[(4,4,5,5,5-pentafluoro-n-pentyl)thio]nonyl]-estra-1,3,5(10)-triene, 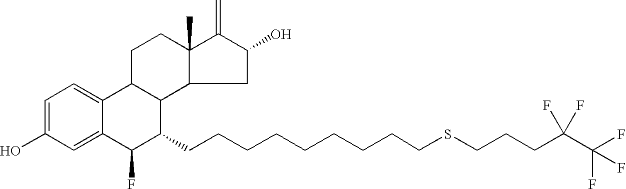
6β-Fluoro-3,16α-dihydroxy-17-methylene-7α-[9-[(4,4,5,5,5-pentafluoro-n-pentyl)sulfinyl]nonyl]-estra-1,3,5(10)-triene, 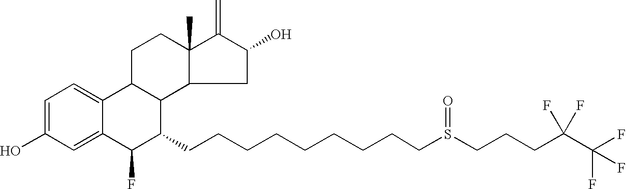
6β-Fluoro-3,16α-dihydroxy-17-methylene-7α-[5-[N-methyl-N-3-(4,4,5,5,5-pentafluoro-n-pentylthio)-propylamino]-pentyl]-estra-1,3,5(10)-triene, 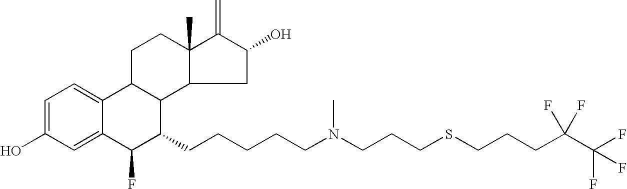
6β-Fluoro-3,16α-dihydroxy-17-methylene-7α-[5-[N-methyl-N-3-(4,4,5,5,5-pentafluoro-n-pentylsulfinyl)-propylamino]-pentyl]-estra-1,3,5(10)-triene, 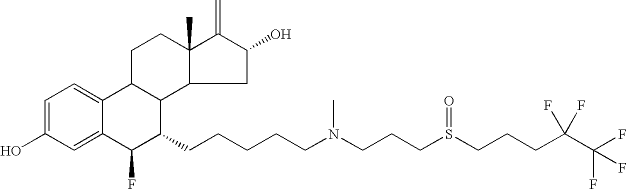
6β-Fluoro-3,16α-dihydroxy-17-methylene-7α-[5-[N-methyl-N-3-(3,3,4,4,5,5,6,6,6-nonafluoro-n-hexyl)-propylamino]-pentyl]-estra-1,3,5(10)-triene, 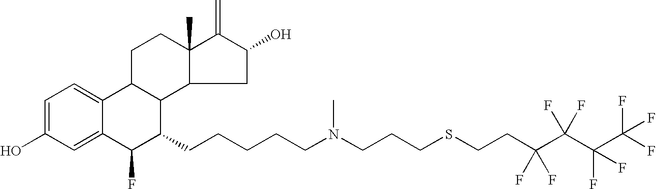
11-(6β-Fluoro-3,16α-dihydroxy-17-methylene-estra-1,3,5(10)-triene-7α-yl)-2-(3,3,4,4,5,5,6,6,6-nonafluoro-n-hexyl)-undecanoic acid, 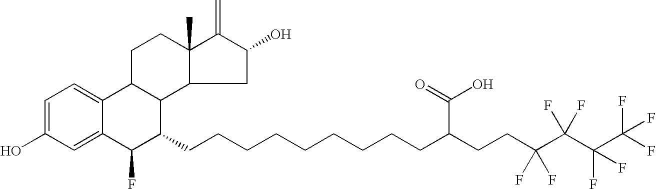
10-(6β-Fluoro-3,16α-dihydroxy-17-methylene-estra-1,3,5(10)-triene-7α-yl)-2-(3,3,4,4,5,5,6,6,6-nonafluoro-n-hexyl)-decanoic acid, 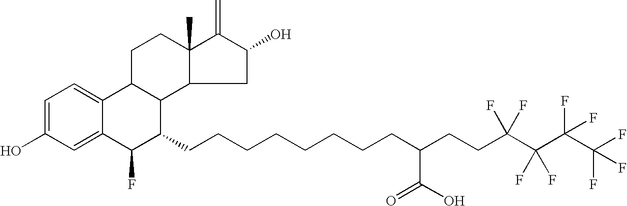
3,6β,16α-Trihydroxy-17-methylene-7α-[9-[(4,4,5,5,5-pentafluoro-n-pentyl)sulfinyl]nonyl]-estra-1,3,5(10)-triene, 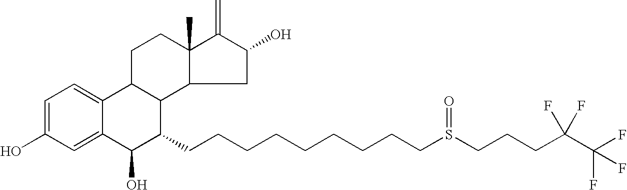
3,6β,16α-Trihydroxy-17-methylene-7α-[5-[N-methyl-N-3-(4,4,5,5,5-pentafluoro-n-pentylthio)-propylamino]-pentyl]-estra-1,3,5(10)-triene, 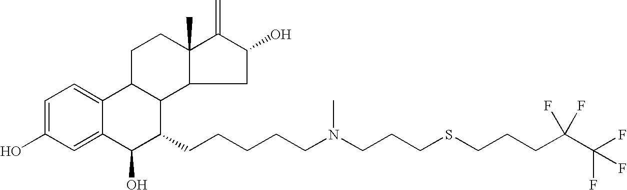
3,6β,16α-Trihydroxy-17-methylene-7α-[5-[N-methyl-N-3-(4,4,5,5,5-pentafluoro-n-pentylsulfinyl)-propylamino]-pentyl]-estra-1,3,5(10)-triene, 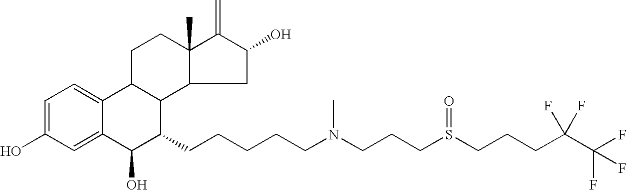
11-(3,6β,16α-Trihydroxy-17-methylene-estra-1,3,5(10)-triene-7α-yl)-2-(3,3,4,4,5,5,6,6,6-nonafluoro-n-hexyl)-undecanoic acid, 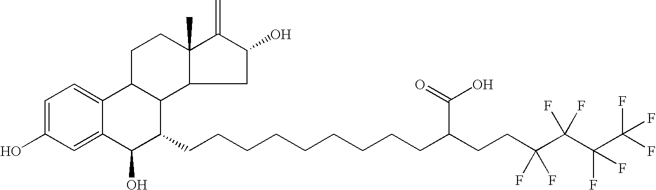
11-(17-(1,2-Ethylene)-3,16α-dihydroxy-estra-1,3,5(10)-triene-7α-yl)-undecanoic acid n-butyl-methyl-amide, 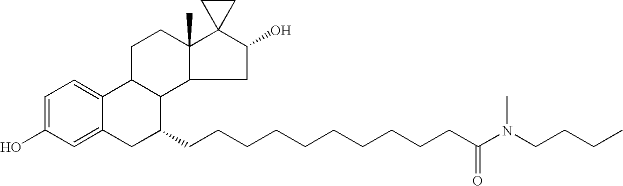
11-(17-(1,2-Ethylene)-3,16α-dihydroxy-estra-1,3,5(10)-triene-7α-yl)-undecanoic acid n-butyl-methyl-amide 3-O-benzoate, 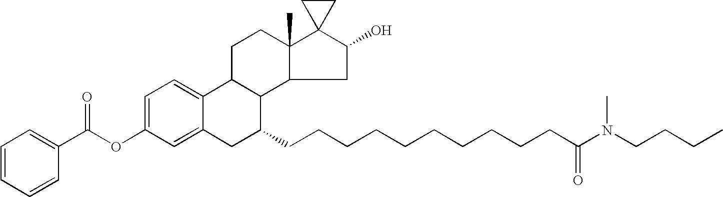
11-(17-(1,2-Ethylene)-3,16α-dihydroxy-estra-1,3,5(10)-triene-7α-yl)-undecanoic acid (2,2,3,3,4,4,4-hepta-fluoro)-n-butyl-methyl-amide, 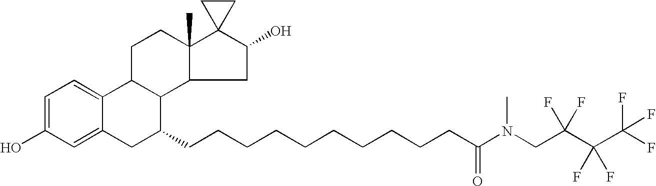
17-(1,2-Ethylene)-3,16α-dihydroxy-7α-[9-[(4,4,5,5,5-pentafluoro-n-pentyl)thio]nonyl]-estra-1,3,5(10)-triene, 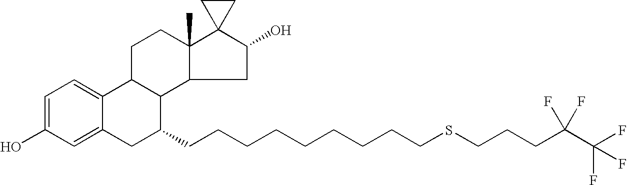
17-(1,2-Ethylene)-3,16α-dihydroxy-7α-[9-[(4,4,5,5,5-pentafluoro-n-pentyl)sulfinyl]nonyl]-estra-1,3,5(10)-triene, 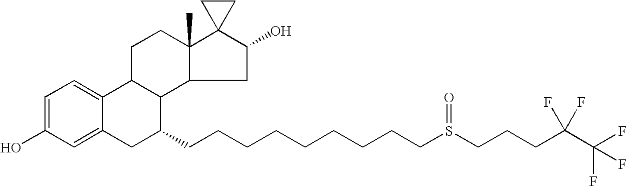
17-(1,2-Ethylene)-3,16α-dihydroxy-7α-[9-[(4,4,5,5,5-pentafluoro-n-pentyl)sulfinyl]nonyl]-estra-1,3,5(10)-triene 3-O-acetate, 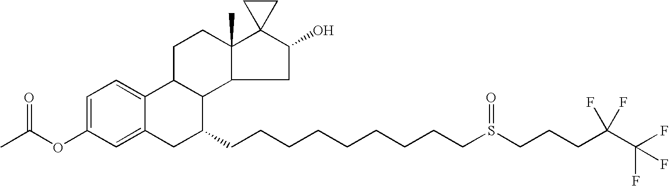
17-(1,2-Ethylene)-3,16α-dihydroxy-7α-[9-[(4,4,5,5,5-pentafluoro-n-pentyl)sulfinyl]nonyl]-estra-1,3,5(10)-triene 3-O-sulfamate, 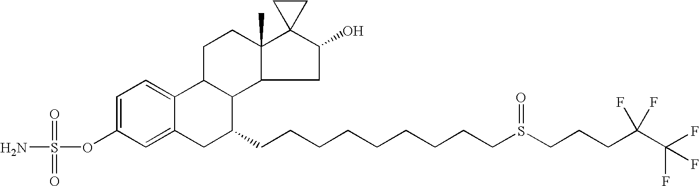
17-(1,2-Ethylene)-3,16α-dihydroxy-7α-[9-[(4,4,5,5,5-pentafluoro-n-pentyl)sulfonyl]nonyl]-estra-1,3,5(10)-triene, 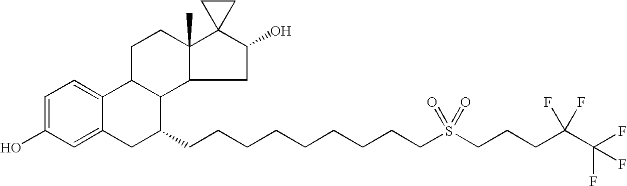
17-(1,2-Ethylene)-3,16α-dihydroxy-7α-[9-[(4,4,5,5,5-pentafluoro-n-pentyl)sulfinyl]octyl]-estra-1,3,5(10)-triene, 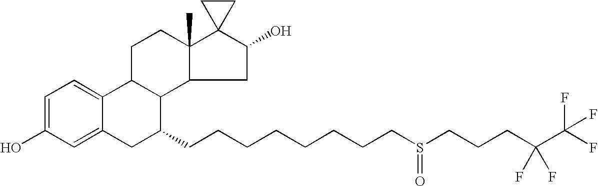
17-(1,2-Ethylene)-7α-[9-[(2,2,3,3,4,4,4-heptafluoro-n-butyl)sulfinyl]nonyl]-3,16α-dihydroxy-estra-1,3,5(10)-triene, 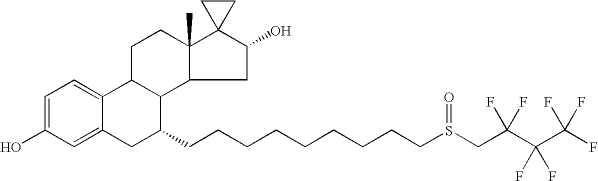
17-(1,2-Ethylene)-3,16α-dihydroxy-7α-[9-[(3,3,4,4,5,5,6,6,6-nonafluoro-n-hexyl) sulfonyl]nonyl]-estra-1,3,5(10)-triene, 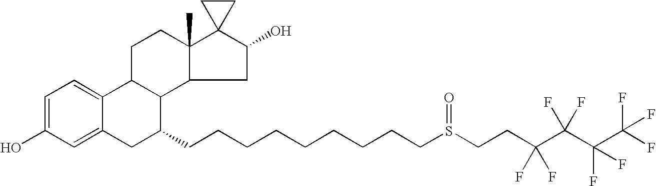
17-(1,2-Ethylene)-3,16α-dihydroxy-7α-[9-[(4,4,5,5,6,6,7,7,7-nonafluoro-n-heptyl) sulfonyl]nonyl]-estra-1,3,5(10)-triene, 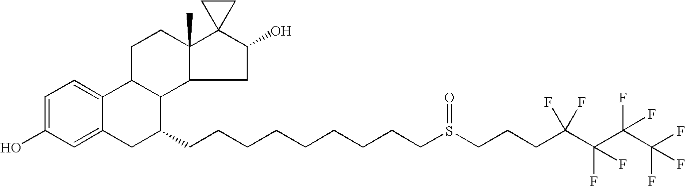
17-(1,2-Ethylene)-3,16α-dihydroxy-7α-[5-[N-methyl-N-3-(4,4,5,5,5-pentafluoro-n-pentylthio)-propylamino]-pentyl]-estra-1,3,5(10)-triene, 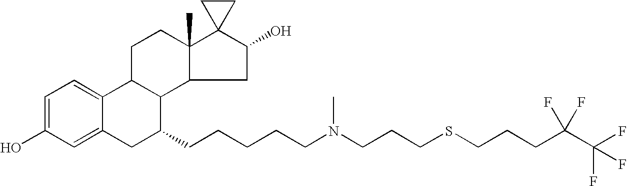
17-(1,2-Ethylene)-3,16α-dihydroxy-7α-[5-[N-methyl-N-3-(4,4,5,5,5-pentafluoro-n-pentylthio)-propylamino]-pentyl]-estra-1,3,5(10)-triene 3-O-benzoate, 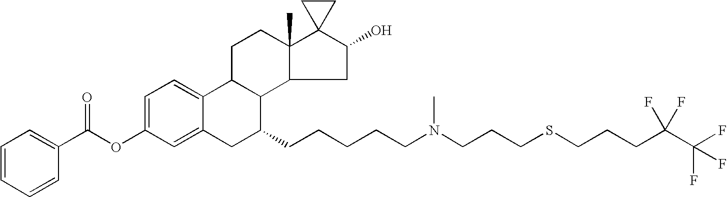
17-(1,2-Ethylene)-3,16α-dihydroxy-7α-[5-[N-methyl-N-3-(4,4,5,5,5-pentafluoro-n-pentylthio)-propylamino]-pentyl]-estra-1,3,5(10)-triene 3-O-acetate, 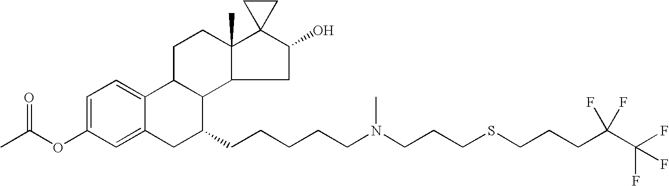
17-(1,2-Ethylene)-3,16α-dihydroxy-7α-[5-[N-methyl-N-3-(4,4,5,5,5-pentafluoro-n-pentylthio)-propylamino]-pentyl]-estra-1,3,5(10)-triene 3-O-sulfamate, 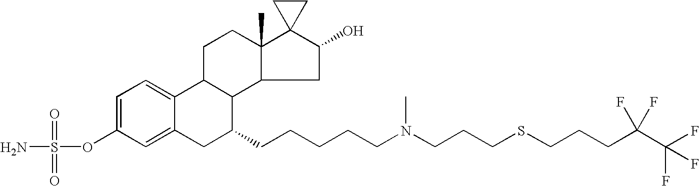
17-(1,2-Ethylene)-3,16α-dihydroxy-7α-[5-[N-methyl-N-3-(4,4,5,5,5-pentafluoro-n-pentylsulfinyl)-propylamino]-pentyl]-estra-1,3,5(10)-triene, 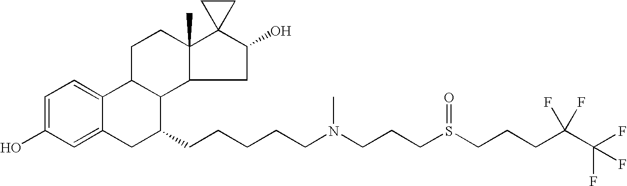
17-(1,2-Ethylene)-3,16α-dihydroxy-7α-[5-[N-methyl-N-3-(4,4,5,5,5-pentafluoro-n-pentylsulfonyl)-propylamino]-pentyl]-estra-1,3,5(10)-triene, 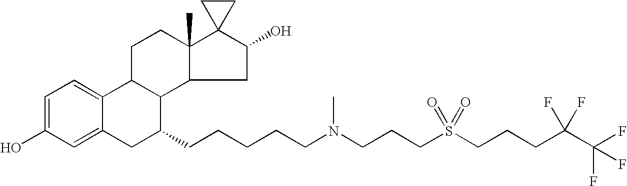
17-(1,2-Ethylene)-3,16α-dihydroxy-7α-[5-[N-methyl-N-3-(3,3,4,4,5,5, 6,6,6-nonafluoro-n-hexyl)-propylamino]-pentyl]-estra-1,3,5(10)-triene, 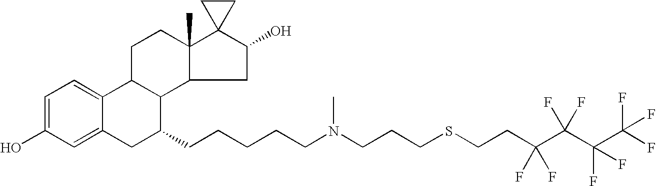
17-(1,2-Ethylene)-3,16α-dihydroxy-7α-[5-[N-methyl-N-3-(4,4,5,5,6,6,7,7,7-nonafluoro-n-heptyl)-propylamino]-pentyl]-estra-1,3,5(10)-triene, 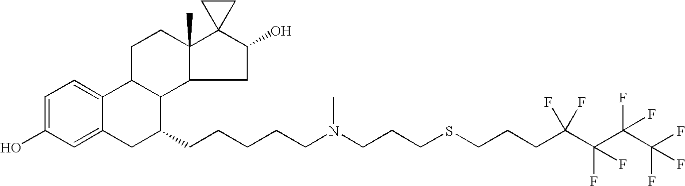
11-(17-(1,2-Ethylene)-3,16α-dihydroxy-estra-1,3,5(10)-triene-7α-yl)-2-(4,4,5,5,5-pentafluoro-n-pentyl)-undecanoic acid, 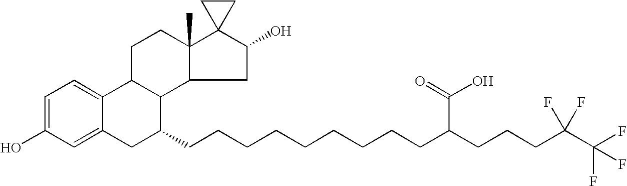
11-(17-(1,2-Ethylene)-3,16α-dihydroxy-estra-1,3,5(10)-triene-7α-yl)-2-(4,4,5,5,6,6,7,7,7-nonafluoro-n-heptyl)-undecanoic acid, 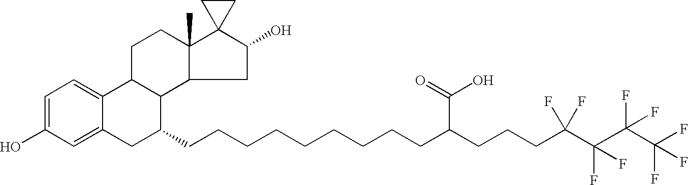
11-(17-(1,2-Ethylene)-3,16α-dihydroxy-estra-1,3,5(10)-triene-7α-yl)-2-(3,3,4,4,5,5,6,6,6-nonafluoro-n-hexyl)-undecanoic acid, 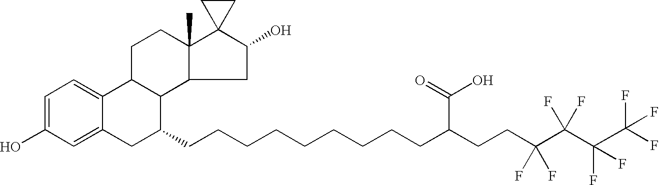
10-(17-(1,2-Ethylene)-3,16α-dihydroxy-estra-1,3,5(10)-triene-7α-yl)-2-(3,3,4,4,5,5,6,6,6-nonafluoro-n-hexyl)-decanoic acid, 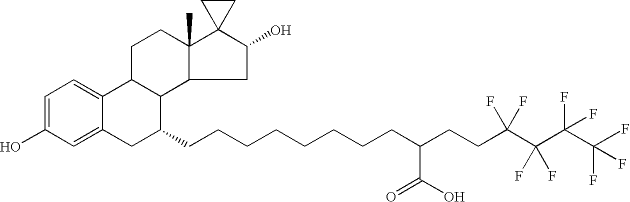
11-(17-(1,2-Ethylene)-3,16α-dihydroxy-estra-1,3,5(10)-triene-7α-yl)-2-(3,3,4,4,5,5,6,6,6-nonafluoro-n-hexyl)-undecanoic acid methylester, 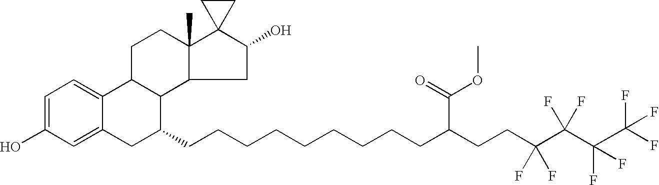
2-[9-(17-(1,2-Ethylene)-3,16α-dihydroxy-estra-1,3,5(10)-triene-7α-yl)-nonyl]-2-(3,3,4,4,5,5,6,6,6-nonafluoro-n-hexyl)-malonic acid, 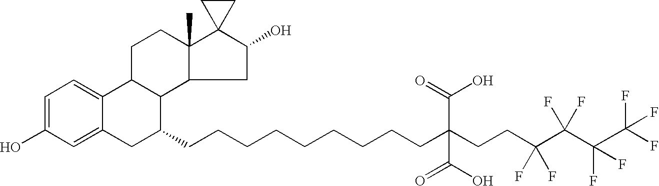
11-(17-(1,2-Ethylene)-3,6α,6α-trihydroxy-estra-1,3,5(10)-triene-7α-yl)-undecanoic acid n-butyl-methyl-amide, 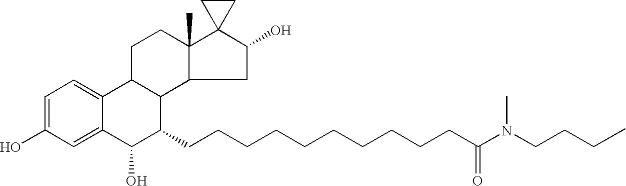
11-(17-(1,2-Ethylene)-3,6α,6α-trihydroxy-estra-1,3,5(10)-triene-7α-yl)-undecanoic acid (2,2,3,3,4,4,4-hepta-fluoro)-n-butyl-methyl-amide, 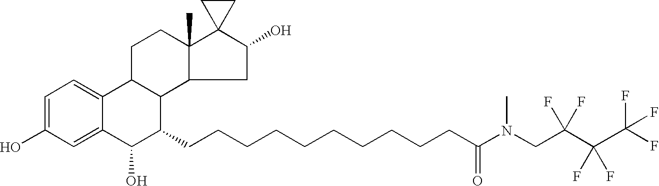
17-(1,2-Ethylene)-3,6α,6α-trihydroxy-7α-[9-[(4,4,5,5,5-pentafluoro-n-pentyl)thio]nonyl]-estra-1,3,5(10)-triene, 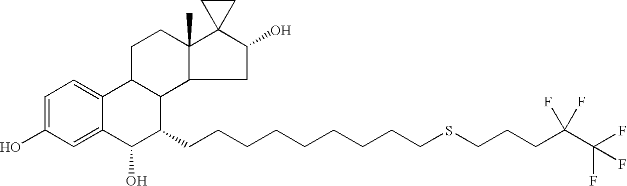
17-(1,2-Ethylene)-3,6α,6α-trihydroxy-7α-[9-[(4,4,5,5,5-pentafluoro-n-pentyl)sulfinyl]nonyl]-estra-1,3,5(10)-triene, 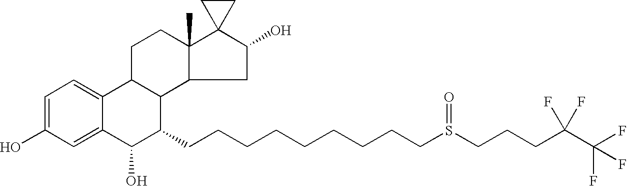
17-(1,2-Ethylene)-3, 6α,6α-trihydroxy-7α-[9-[(4,4,5,5,5-pentafluoro-n-pentyl)sulfinyl]nonyl]-estra-1,3,5(10)-triene 3-O-sulfamate, 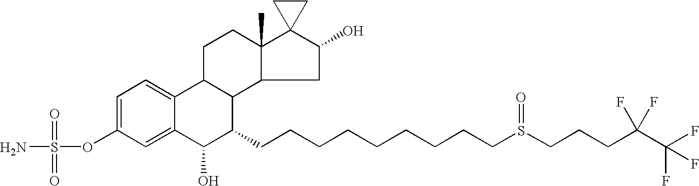
17-(1,2-Ethylene)-3,6α,6α-trihydroxy-7α-[9-[(4,4,5,5,5-pentafluoro-n-pentyl)sulfonyl]nonyl]-estra-, 1,3,5(10)-triene, 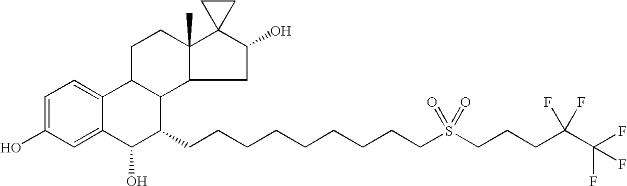
17-(1,2-Ethylene)-7α-[9-[(2,2,3,3,4,4,4-heptafluoro-n-butyl)sulfinyl]nonyl]-3,6α,6α-trihydroxy-estra-1,3,5(10)-triene, 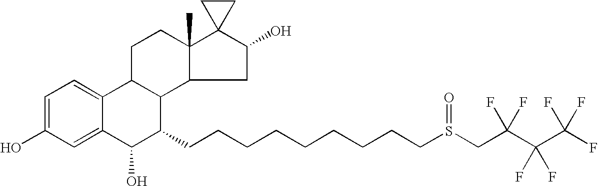
17-(1,2-Ethylene)-3,6α,6α-trihydroxy-7α-[5-[N-methyl-N-3-(4,4,5,5,5-pentafluoro-n-pentylthio)-propylamino]-pentyl]-estra-1,3,5(10)-triene, 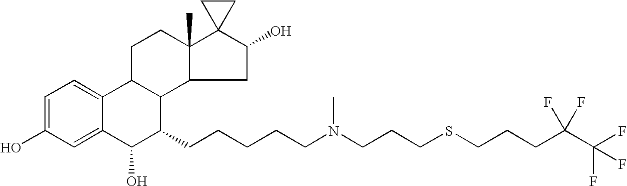
17-(1,2-Ethylene)-3,6α,6α-trihydroxy-7α-[5-[N-methyl-N-3-(4,4,5,5,5-pentafluoro-n-pentylthio)-propylamino]-pentyl]-estra-1,3,5(10)-triene 3-O-sulfamate, 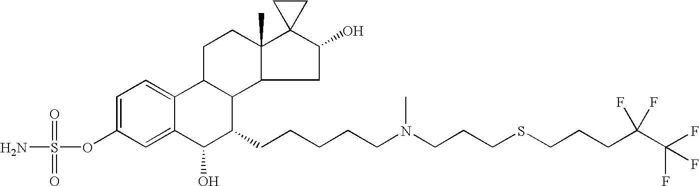
17-(1,2-Ethylene)-3,6α, 6α-trihydroxy-7α-[5-[N-methyl-N-3-(4,4,5,5,5-pentafluoro-n-pentylsulfinyl)-propylamino]-pentyl]-estra-1,3,5(10)-triene, 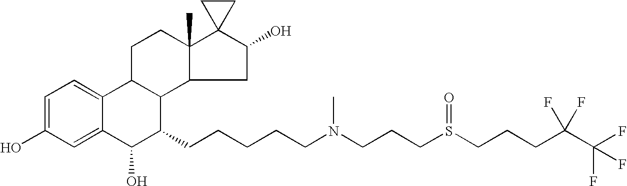
17-(1,2-Ethylene)-3,6α,6α-trihydroxy-7α-[5-[N-methyl-N-3-(4,4,5,5,5-pentafluoro-n-pentylsulfonyl)-propylamino]-pentyl]-estra-1,3,5(10)-triene, 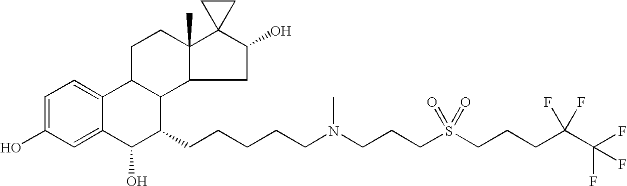
11-(17-(1,2-Ethylene)-3,6α,6(-trihydroxy-estra-1,3,5(10)-triene-7α-yl)-2-(4,4,5,5,5-pentafluoro-n-pentyl)-unde-canoic acid, 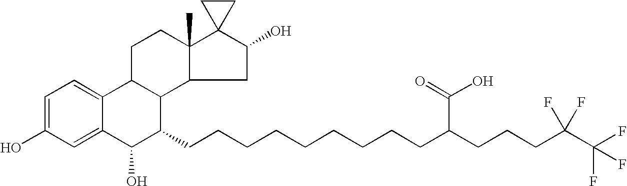
11-(17-(1,2-Ethylene)-3,6α,6(-trihydroxy-estra-1,3,5(10)-triene-7α-yl)-2-(4,4,5,5,6,6,7,7,7-nonafluoro-n-heptyl)-undecanoic acid, 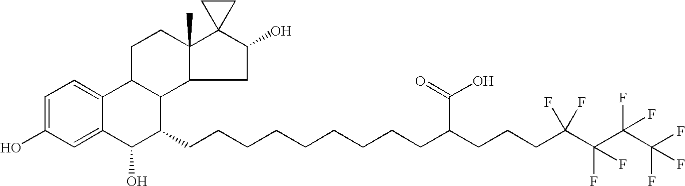
10-(17-(1,2-Ethylene)-3,6α,6(-trihydroxy-estra-1,3,5(10)-triene-7α-yl)-2-(3,3,4,4,5,5,6,6,6-nonafluoro-n-hexyl)-decanoic acid, 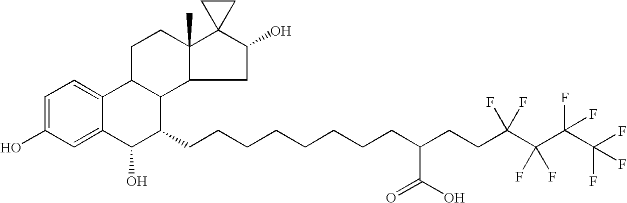
11-(17-(1,2-Ethylene)-3,6α,6α-trihydroxy-estra-1,3,5(10)-triene-7α-yl)-2-(4,4,5,5,5-pentafluoro-n-pentyl)-undecanoic acid methylester, 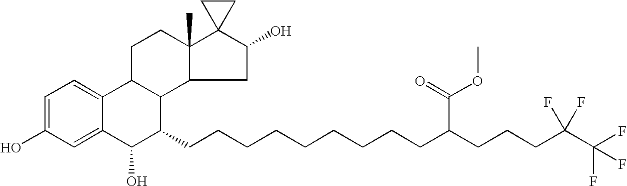
11-(17-(1,2-Ethylene)-3,6α, 6α-trihydroxy-estra-1,3,5(10)-triene-7α-yl)-2-(4,4,5,5,6,6,7,7,7-nonafluoro-n-heptyl)-undecanoic acid methylester, 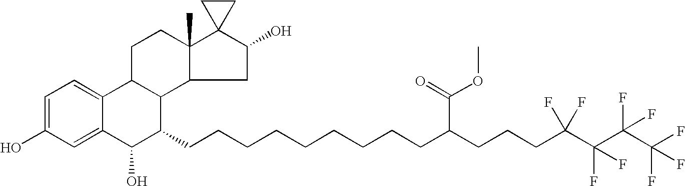
2-[9-(17-(1,2-Ethylene)-3,6α,6α-trihydroxy-estra-1,3,5(10)-triene-7α-yl)-nonyl]-2-(3,3,4,4,5,5,6,6,6-nonafluoro-n-hexyl)-malonic acid, 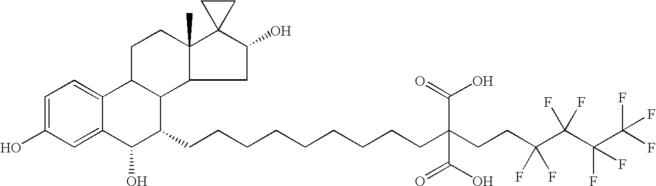
11-(17-(1,2-Ethylene)-6β-fluoro-3,16α-dihydroxy-estra-1,3,5(10)-triene-7α-yl)-undecanoic acid n-butyl-methyl-amide, 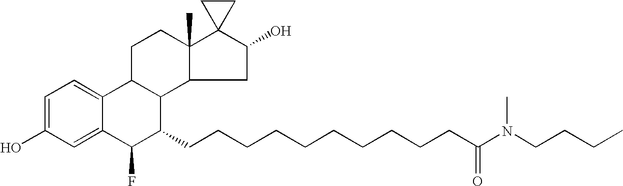
11-(17-(1,2-Ethylene)-6β-fluoro-3,16α-dihydroxy-estra-1,3,5(10)-triene-7α-yl)-undecanoic acid (2,2,3,3,4,4,4-heptafluoro)-n-butyl-methyl-amide, 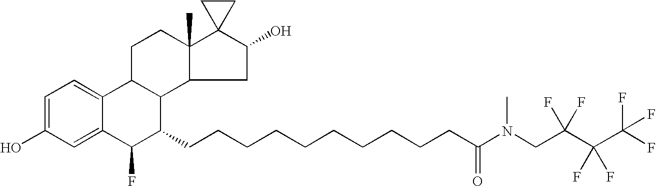
17-(1,2-Ethylene)-6β-fluoro-3,16α-dihydroxy-7α-[9-[(4,4,5,5,5-pentafluoro-n-pentyl) thio]nonyl]-estra-1,3,5 (10)-triene, 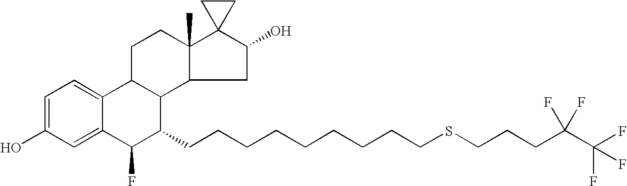
17-(1,2-Ethylene)-6β-fluoro-3,16α-dihydroxy-7α-[9-[(4,4,5,5,5-pentafluoro-n-pentyl) sulfinyl]nonyl]-estra-1,3,5(10)-triene, 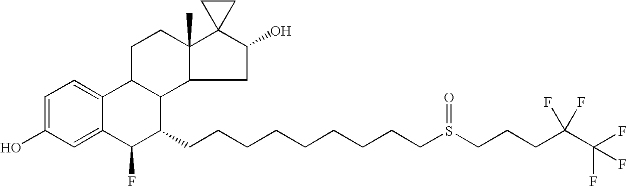
17-(1,2-Ethylene)-6β-fluoro-3,16α-dihydroxy-7α-[9-[(4,4,5,5,5-pentafluoro-n-pentyl) sulfinyl]nonyl]-estra-1,3,5(10)-triene 3-O-sulfamte, 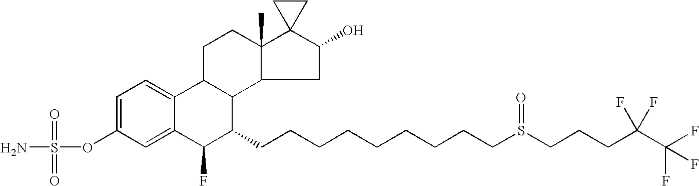
17-(1,2-Ethylene)-6β-fluoro-3,16α-dihydroxy-7α-[9-[(4,4,5,5,5-pentafluoro-n-pentyl) sulfonyl]nonyl]-estra-1,3,5(10)-triene, 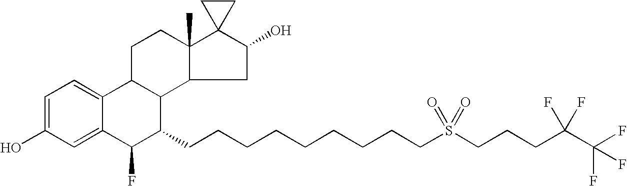
17-(1,2-Ethylene)-6β-fluoro-3,16α-dihydroxy-7α-[5-[N-methyl-N-3-(4,4,5,5,5-pentafluoro-n-pentylthio)-propyl-amino]-pentyl]-estra-1,3,5(10)-triene, 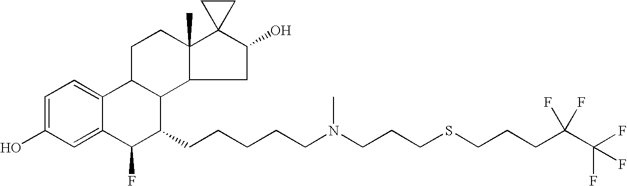
17-(1,2-Ethylene)-6β-fluoro-3,16α-dihydroxy-7α-[5-[N-methyl-N-3-(4,4,5,5,5-pentafluoro-n-pentylthio)-propyl-amino]-pentyl]-estra-1,3,5(10)-triene 3-O-sulfamate, 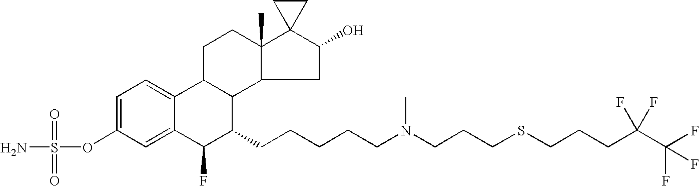
17-(1,2-Ethylene)-6β-fluoro-3,16α-dihydroxy-7α-[5-[N-methyl-N-3-(4,4,5,5,5-pentafluoro-n-pentylsulfinyl)-propylamino]-pentyl]-estra-1,3,5(10)-triene, 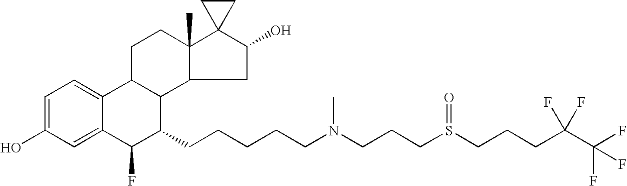
17-(1,2-Ethylene)-6β-fluoro-3,16α-dihydroxy-7α-[5-[N-methyl-N-3-(4,4,5,5,5-pentafluoro-n-pentylsulfonyl)-propylamino]-pentyl]-estra-1,3,5(10)-triene, 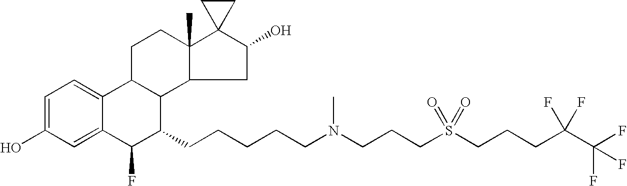
11-(17-(1,2-Ethylene)-6β-fluoro-3,16α-dihydroxy-estra-1,3,5(10)-triene-7α-yl)-2-(4,4,5,5,5-pentafluoro-n-pentyl)-undecanoic acid, 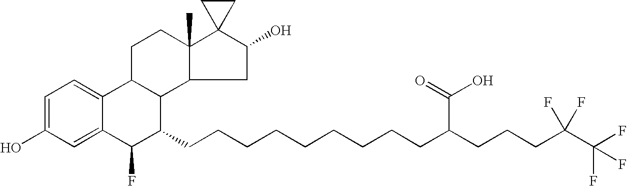
11-(17-(1,2-Ethylene)-6β-fluoro-3,16α-dihydroxy-estra-1,3,5(10)-triene-7α-yl)-2-(4,4,5,5,6,6,7,7,7-nonafluoro-n-heptyl)-undecanoic acid, 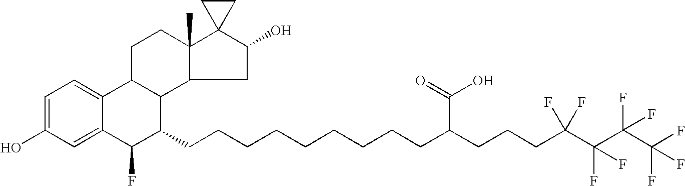
11-(17-(1,2-Ethylene)-6β-fluoro-3,16α-dihydroxy-estra-1,3,5(10)-triene-7α-yl)-2-(4,4,5,5,6,6,7,7,7-nonafluoro-n-heptyl)-undecanoic acid methylester, 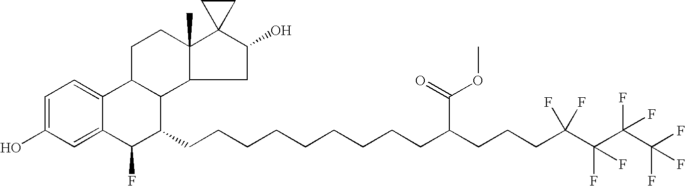
17-(1,2-Ethylene)-3,6β,6α-trihydroxy-7α-[9-[(4,4,5,5,5-pentafluoro-n-pentyl)thio]nonyl]-estra-1,3,5(10)-triene, 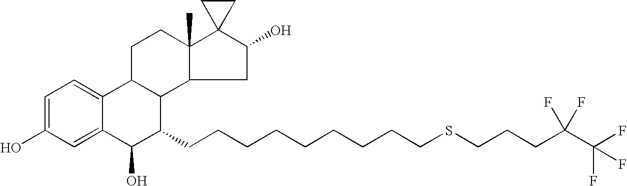
17-(1,2-Ethylene)-3,6β,6α-trihydroxy-7α-[9-[(4,4,5,5,5-pentafluoro-n-pentyl)sulfinyl]nonyl]-estra-1,3,5(10)-triene, 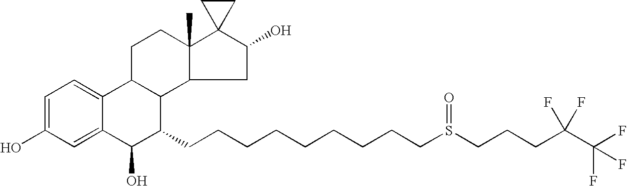
17-(1,2-Ethylene)-3,6β,6α-trihydroxy-7α-[5-[N-methyl-N-3-(4,4,5,5,5-pentafluoro-n-pentylthio)-propylamino]-pentyl]-estra-1,3,5(10)-triene, 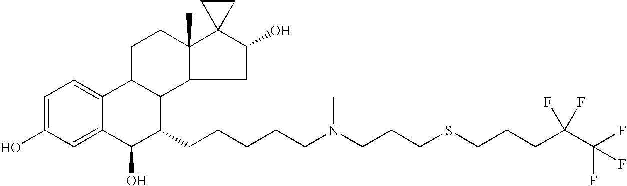
17-(1,2-Ethylene)-3,6β,6α-trihydroxy-7α-[5-[N-methyl-N-3-(4,4,5,5,5-pentafluoro-n-pentylsulfinyl)-propylamino]-pentyl]-estra-1,3,5(10)-triene, 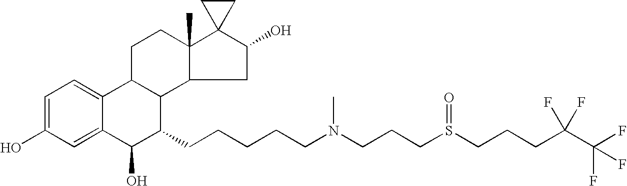
11-(17-(1,2-Ethylene)-3,6β,6α-trihydroxy-estra-1,3,5(10)-triene-7α-yl)-2-(4,4,5,5,5-pentafluoro-n-pentyl)-undecanoic acid, 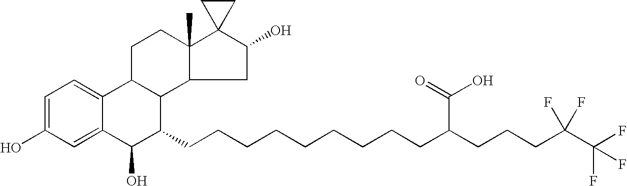
11-(17-(1,2-Ethylene)-3,6β,6α-trihydroxy-estra-1,3,5(10)-triene-7α-yl)-2-(4,4,5,5,6,6,7,7,7-nonafluoro-n-heptyl)-undecanoic acid, 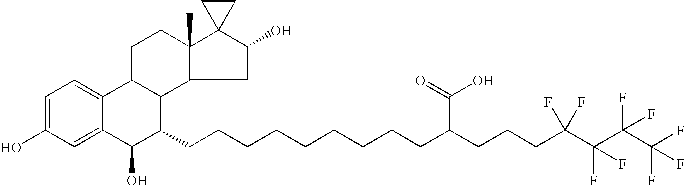
17-(1,2-Ethylene)-3,16α-dihydroxy-6-keto-7α-[9-(4,4,5,5,5-pentafluoro-n-pentyl)thiononyl]-estra-1,3,5(10)-triene, 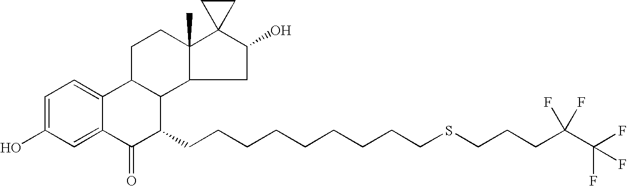
17-(1,2-Ethylene)-3,16α-dihydroxy-6-keto-7α-[9-[(4,4,5,5,5-pentafluoro-n-pentyl) sulfinyl]nonyl]-estra-1,3,5(10)-triene, 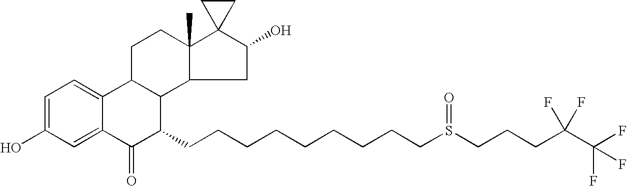
17-(1,2-Ethylene)-3,16α-dihydroxy-6-keto-7α-[5-[N-methyl-N-3-(4,4,5,5,5-pentafluoro-n-pentylthio)-propylamino]-pentyl]-estra-1,3,5(10)-triene, 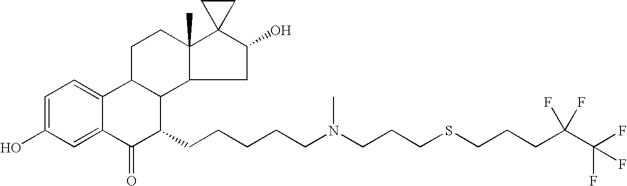
17-(1,2-Ethylene)-3,16α-dihydroxy-6α-methoxy-7α-[9-(4,4,5,5,5-pentafluoro-n-pentyl)thiononyl]-estra-1,3,5(10)-triene, 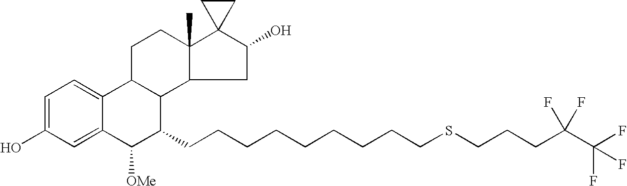
17-(1,2-Ethylene)-3,16α-dihydroxy-6α-methoxy-7α-[9-[(4,4,5,5,5-pentafluoro-n-pentyl) sulfinyl]nonyl]-estra-1,3,5(10)-triene, 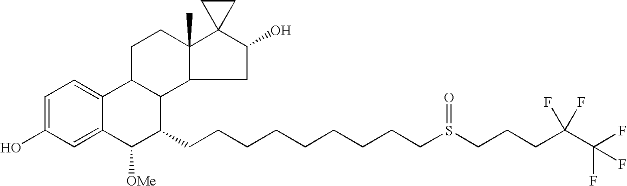
17-(1,2-Ethylene)-3,16α-dihydroxy-6β-methoxy-7(-[9-(4,4,5,5,5-pentafluoro-n-pentyl)thiononyl]-estra-1,3,5(10)-triene, 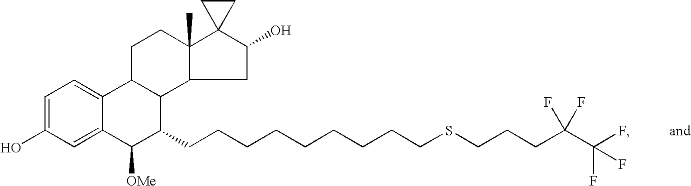
17-(1,2-Ethylene)-3,16α-dihydroxy-6β-methoxy-7α-[9-[(4,4,5,5,5-pentafluoro-n-pentyl) sulfinyl]nonyl]-estra-1,3,5(10)-triene

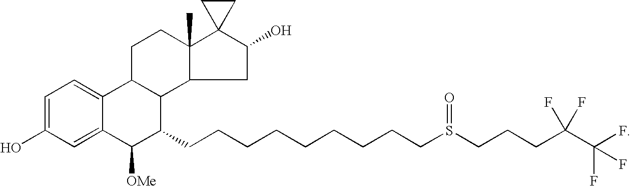

In a further aspect, the invention relates to an intermediate compound of the general formula VIII:

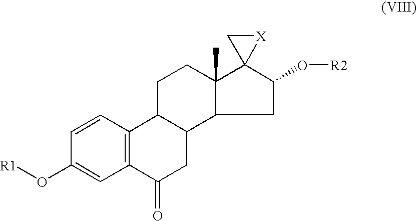

wherein R1, R2 and X are as defined above.

In a second aspect the present invention relates to a new compound as described above for use as a medicament.

In a third aspect the present invention relates to the use of a new compound as described above for the manufacturing of a medicament for the treatment of an estrogen related disorder or condition that benefits from antiestrogen treatment.

In one preferred embodiment the estrogen related disorder or condition is chosen from the group comprising estrogen dependent breast cancer, anovulatory infertility, menstrual disorders, male pattern baldness, dysfunctional uterine bleeding, endometrial polyps, benign breast disease, uterine leiomyomas, adenomyosis, ovarian cancer, endometrial cancer, melanoma, prostate cancer, cancers of the colon, CNS cancers, endometriosis, polycystic ovary syndrome, infertility and contraception in males.

In another preferred embodiment the estrogen related disorder is estrogen dependent breast cancer.

In a fourth aspect the present invention relates to a pharmaceutical composition comprising a new compound as described above admixed with one or more pharmaceutically acceptable excipients or carriers.

In one preferred embodiment the excipients are chosen from the group comprising filling agents, lubricants, flavours, colourings, sweetenings, buffers, acidifying agents, diluents and preservatives.

In another prefered embodiment the pharmaceutical composition is administered orally, intramuscularly, intravenously, intraperitoneally or subcutaneously, via implants, rectally, intranasally, transdermally, or vaginally, preferably orally, transdermally or intranasally.

In a fifth aspect the present invention relates to a method of treatment comprising administration of a pharmaceutically effective amount of a new compound as described above or a pharmaceutical composition as described above to a subject suffering from an estrogen dependent disorder or condition.

In one embodiment the estrogen dependent disorder or condition to be treated is chosen from the group comprising estrogen dependent breast cancer, anovulatory infertility, menstrual disorders, male pattern baldness, dysfunctional uterine bleeding, endometrial polyps, benign breast disease, uterine leiomyomas, adenomyosis, ovarian cancer, endometrial cancer, melanoma, prostate cancer, cancers of the colon, CNS cancers, endometriosis, polycystic ovary syndrome, infertility and contraception in males.

In another preferred embodiment the estrogen dependent disorder is estrogen dependent breast cancer.

The compounds of the present invention may be given in doses about 0.1-1000 mg/day, preferably in doses about 1-100 mg/day. The compounds of the present invention may be administered orally, by injections, e.g. intramuscular, intravenous, intraperitoneal, or subcutaneous, via implants, rectally, intranasally, transdermally, vaginally or by any other route suitable to deliver an therapeutically active amount of the compound.

The pharmaceutical composition of the present invention comprises a pharmaceutically effective dose of at least one of the compounds according to the present invention, preferably in admixture with one or more pharmaceutically acceptable excipients, diluents or carriers. The amount administered will vary depending on various factors, eg age, sex, weight, which disorder or condition that is treated and the compound used. Both local and systemic administration is possible.

With “pharmaceutically acceptable” is meant that the excipients, diluents or carriers must be compatible with the other ingredients of the formulation, and not deleterious to the receipient thereof.

The pharmaceutical composition can be prepared according to any of the methods well known by a person skilled in the art of pharmacy. Such methods may include the step of bringing the novel compounds of the present invention in contact with liquid carriers, solid matrices, semi-solid carriers, finely diveded solid carriers or combinations thereof, and then, if necessary, introducing or shaping the product into the desired delivery system.

One or more suitable unit dosage forms comprising a pharmaceutically effective dose of at least one of the compounds according to the present invention, optionally formulated for sustained release, can be administered by a variety of routes e. g. orally, intramuscularly, intravenously, intraperitoneally or subcutaneously, via implants, rectally, intranasally, transdermally, or vaginally. Preferably, the novel compounds according to the invention are administrated orally, transdermally or intranasally.

The invention is also intended to encompass pro-drugs of the compounds with formula I which are transformed into compounds with formula I in vivo (under physiological conditions or via metabolic pathways). Prodrugs may show improved effects as regards uptake, stability, hydrophilicity/hydrophobicity, chemical stability or delayed/prolonged release. Suitable pro-drugs and their methods of manufacture are known in the literature and will be routine for persons skilled in the art. A simple example of a pro-drug might be an alkyl ester of an alcohol functionality, as ester groups are known to hydrolyse under physiological conditions.

### EMBODIMENTS OF THE PRESENT INVENTION

The present invention will now be described in more detail by the following examples, which are included in order to disclose some embodiments of the invention, but not in any way to limit the scope of the invention.

In the description of the preparative methods, the manipulation of protecting groups is not included. It is obvious for the person skilled in the art that some functional groups, e.g. hydroxy groups, need to be protected, e.g. as acetals, ethers, or silyl ethers, during the synthetic steps.

The novel steroidal anti-estrogens according to the invention can be prepared from 7α-substituted estradiol or estrone derivatives by methods described in the literature (Scheme 1, WO9708188).

The 7α-substituted estradiol or estrone derivatives can be prepared by nucleophilic addition to steroidal 6-en derivatives or by alkylating 6-keto-estra-1,3,5(10)-triene derivaties with electrophilic reagents (ref 6). 6-Keto-derivatives can be prepared by oxidation methods descibed in the literature, e.g. the 2 step procedure using H₂O₂ and PCC as oxidizing agents (ref 6).

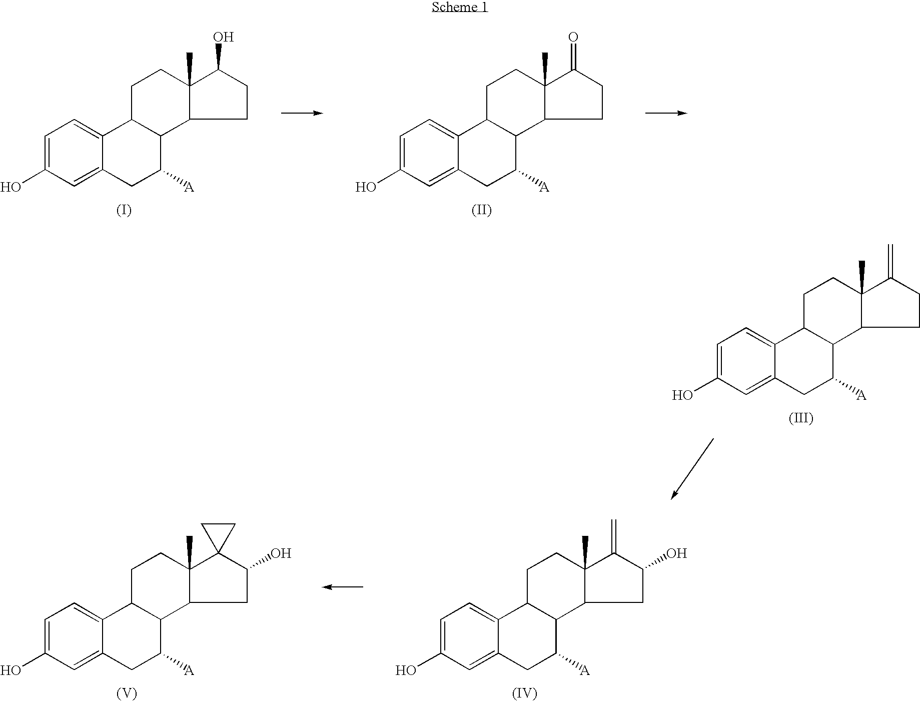

Thus, the 7α-substituted estradiol derivative (I) may be oxidized to the estrone derivative (II) by known methods, e.g. by pyridinium chlorochromate (PCC) or tetrapropylammonium perruthenate/N-methylmorpholine N-oxide (TPAP/NMNO) in inert solvents like CH₂Cl₂. The estrone derivative (II) may be reacted with a Wittig-type reagent, like Ph₃PCH₂, preferably in DMSO or toluene as solvent, to give the exo-methylene derivative (III).

Allylic oxidation of (III) by SeO₂ then stereoselectively gives the 17-methylene-16α-hydroxy derivative (IV). This can also be prepared from 16α-hydroxy-17-one derivatives by Wittig-type reactions, e.g. using the Tebbe reagent. Cyclopropanation of (IV) to give the 17-(1′,2′-ethylene)-16α-hydroxy derivative (V) may be accomplished by Simmons-Smith like reagents, e.g. by CH₂I₂/ZnEt₂ in CH₂Cl₂.

Alternatively, the manipulation of the D-ring can be done prior to the introduction of the 7α-side chain (Scheme 2) using the same methods as described above.

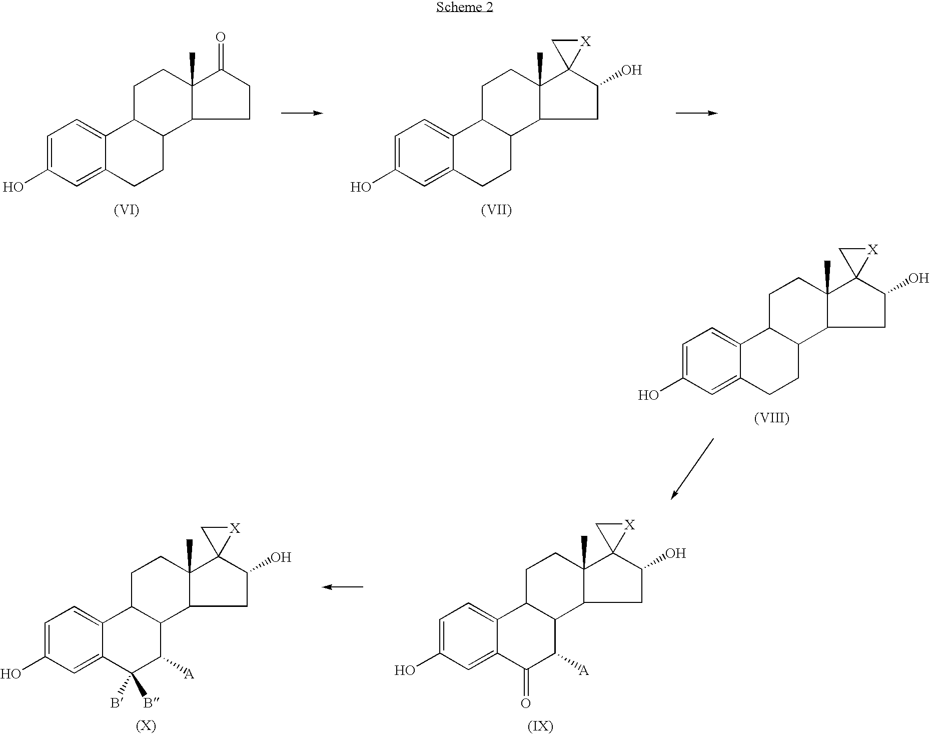

The 17-alkylene-16α-hydroxy derivative (VII) can be oxidized to give the 6-keto derivative (VIII), which may be 7α-alkylated to give (IX), e.g. by reacting the enolate of (VIII) with alkyl iodides in an inert solvent. Further transformations of (IX) into 6α- or 6β-derivatives may be accomplished by methods known to a person skilled in the art. Thus (IX) can be subjected to reduction methods, e.g. by hydride reagents, to give the 6α-hydroxy derivative (B′=—OH) or the methylene derivative (B′,B″=H,H). The 6α-hydroxy derivative (B′=—OH) may be epimerized by Mitsunobu-reactions to give 6β-hydroxy derivatives. The 6α-hydroxy derivative can also be transformed into 6-halo derivatives, e. g. by thionyl chloride or by the DAST reagent, or reduced to the methylene derivative by, e.g. hydride reagents like Et₃SiH or Bu₃SnH under acidic or radical-initiated conditions. The 6-halo derivatives can be reacted with nucleophiles, e.g. hydride reagents like LiEt₃BH to give the methylene derivative or with alcohols to give 6-alkoxy derivatives.

In the preparative examples column chromatography separations were performed using Merck SiO₂ 60 (0.040-0.063 mm) silica gel. TLC analyses were performed on Merck SiO₂ 60 F254 precoated aluminium sheets and the spots were visualized by charring with 10% aqueous H₂SO₄. Microwave-assisted reactions were performed in sealed tubes using a PersonalChemistry Smith Synthesizer. MS spectra were recorded with a ThermoFinnigan LCQ. NMR spectra were recorded with a Bruker ARX 400 (400 MHz) with TMS as internal standard.

Preparation of Starting Materials (SM)

SM1

### 11-Iodo-undecanoic acid n-butyl-methyl-amide

### a. 11-Bromo-undecanoic acid n-butyl-methyl-amide

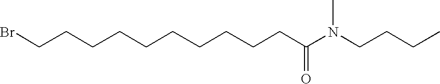

n-Butylmethylamine (1.31 g, 15.0 mmol) was added to a solution of 11-bromo-undecanoic acid (2.65 g, 10.0 mmol), dimethylaminopyridine (DMAP, 0.10 g, 0.82 mmol) and N-(3-dimethylaminopropyl)-N′-ethylcarbodiimide hydrochloride (2.20 g, 11.5 mmol) in CH₂Cl₂ (10 ml). The reaction mixture was stirred for 3 h, concentrated at reduced pressure and purified on column chromatography (heptane-EtOAc, 3:2) to give the title compound (2.75 g, 82%) as an oil.

¹H NMR (CDCl₃) δ 0.93, 0.96 (2t, J=7.3 Hz, 3H), 1.38-1.68 (m, 18H), 1.44-1.63 (m, 4H), 1.86 (p, J=7.2, 2H), 2.29 (m, 2H), 2.91, 2.97 (2s, 3H), 3.26, 3.36 (2t, J=7.6 Hz, 2H) 3.41 (t, J=7.0 Hz, 2H).

### b. 11-Iodo-undecanoic acid n-butyl-methyl-amide

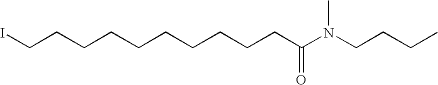

NaI (11.0 g, 73.4 mmol) was added to solution of 11-bromo-undecanoic acid n-butyl-methyl-amide (15.0 g, 44.9 mmol) in acetone (150 ml) under N₂. The solution was stirred at 60° C. over night to give a slurry. Heptane (300 ml) was added and most of the acetone was evaporated. The slurry was filtered through a short column of silica. The silica was washed with heptane/EtoAc (1:1) and the eluate was concentrated at reduced pressure to give the title compound (17.0 g, 99%) as an oil.

¹H NMR (CDCl₃) δ 0.92, 0.95 (2t, J=7.3 Hz, 3H), 1.25-1.42 (m, 14H), 1.44-1.63 (m, 4H), 1.82 (p, J=7.2, 2H), 2.29 (m, 2H), 2.91, 2.96 (2s, 3H), 3.19 (t, J=7.0 Hz, 2H), 3.25, 3.36 (2t, J=7.6 Hz, 2H).

SM2

### 1-Iodo-9-(4,4,5,5,5-pentafluoro-pentylsulfanyl)-nonane

### a. Thiobenzoic acid S-(4,4,5,5,5-pentafluoro-pentyl)ester

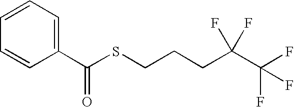

Diisopropyl azodicarboxylate (DIAD, 3.94 ml, 20.0 mmol) was added to a solution of triphenylphosphine (5.25 g, 20.0 mmol) in THF (120 ml) under N₂ at 0° C. After stirring for 30 min a solution of thiobenzoic acid (2.34 ml, 20.0 mmol) and 4,4,5,5,5-pentafluoro-pentanol (1.78 g, 10.0 mmol) in THF (60 ml) was added. The reaction mixture was stirred 0° C. for 1 h and then at room temperature over night. The reaction mixture was concentrated at reduced pressure and was purified on column chromatography (heptane-EtOAc, 20:1) to give the title compound (2.95 g, 99%) as an oil.

Rf (heptane-EtOAc, 20:1)=0.37 ¹H NMR (CDCl₃) δ 1.96-2.05 (m, 2H), 2.11-2.27 (m, 2H), 3.16 (t, J=7.1 Hz, 2H), 7.47 (t, J=7 Hz, 2H), 7.59 (t, J=7 Hz, 1H), 7.97 (t, J=7 Hz, 2H).

### b. 9-(4,4,5,5,5-Pentafluoro-pentylsulfanyl)-1-nonananol

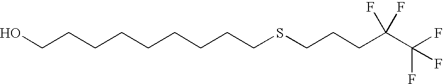

Thiobenzoic acid S-(4,4,5,5,5-pentafluoro-pentyl) ester (8.26 g, 27.7 mmol) was added to a solution of t-BuOK (4.49 g, 40.0 mmol) in MeOH (30 ml). After stirring for 30 min a solution of 9-bromo-1-nonanol (6.18 g, 27.7 mmol) in MeOH (30 ml) was added. The reaction mixture was stirred over night, concentrated at reduced pressure and partitioned between Et₂O and water. The organic phase was washed with water and brine, dried (Na₂SO₄) and concentrated at reduced pressure. The residue was purified on column chromatography (heptane-EtOAc, 3:1) to give the title compound (7.70 g, 83%) as an oil which crystallized on standing.

R_{f} (heptane-EtOAc, 3:1)=0.24 ¹H NMR (CDCl₃) δ 1.28-1.42 (m, 10H), 1.53-1.62 (m, 4H), 1.89 (m, 2H), 2.18 (m, 2H), 2.51 (t, J=7.4 Hz, 2H), 2.59 (t, J=7.0 Hz, 2H), 3.64 (t, J=6.6 Hz, 2H).

### c. Methanesulfonic acid 9-(4,4,5,5,5-pentafluoro-pentyl-sulfanyl)-nonyl ester

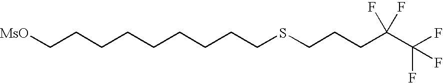

Methanesulphonic acid anhydride (4.35 g, 25.0 mmol) was added to a solution of 9-(4,4,5,5,5-pentafluoro-pentylsulfanyl)-1-nonananol (7.70 g, 22.9 mmol) and EtNiPr₂ (4.28 ml, 25.0 mmol) in CH₂Cl₂ (50 ml). The reaction mixture was stirred for 2 h, concentrated at reduced pressure and purified on column chromatography (heptane-EtOAc, 3:1) to give the title compound (9.42 g, 99%) as an oil which crystallized on standing.

R_{f} (heptane-EtOAc, 3:1)=0.28 ¹H NMR (CDCl₃) δ 1.25-1.45 (m, 10H), 1.53-1.62 (m, 2H), 1.75 (m, 2H), 1.88 (m, 2H), 2.17 (m, 2H), 2.51 (t, J=7.3 Hz, 2H), 2.59 (t, J=7.1 Hz, 2H), 3.00 (s, 3H), 4.22 (t, J=6.6 Hz, 2H).

### d. 1-Iodo-9-(4,4,5,5,5-pentafluoro-pentylsulfanyl)-nonane

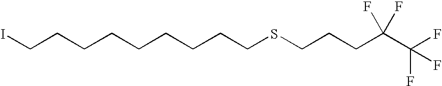

Prepared as described for SM1-b using methanesulfonic acid 9-(4,4,5,5,5-pentafluoro-pentylsulfanyl)-nonyl ester (8.48 g, 20.5 mmol) as starting material to give the title compound (8.93 g, 98%) as an oil.

R_{f} (heptane-EtOAc, 3:1)=0.72 ¹H NMR (CDCl₃) δ 1.25-1.43 (m, 10H), 1.58 (m, 2H), 1.77-1.92 (m, 4H), 2.17 (m, 2H), 2.51 (t, J=7.5 Hz, 2H), 2.59 (t, J=7.0 Hz, 2H), 3.19 (t, J=7.0 Hz, 2H).

SM3

### 1-Methylamino-3-(4,4,5,5,5-pentafluoro-pentylsulfanyl)-propane

### a. Thioacetic acid S-(4,4,5,5,5-pentafluoro-pentyl)ester

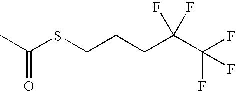

Prepared as described for SM2-a using thioacetic acid (18.2 g, 239 mmol) and 4,4,5,5,5-pentafluoro-pentanol (21.3 g, 120 mmol) as starting materials. The crude product was purified by distillation (b.p. 68° C./20 mmHg, 19.9 g, 70%).

¹H NMR (CDCl₃) δ 1.89 (m, 2H), 2.10 (m, 2H), 2.35 (s, 3H), 2.95 (t, J=7.0 Hz, 2H).

### b. 1-Chloro-3-(4,4,5,5,5-pentafluoro-pentylsulfanyl)-propane

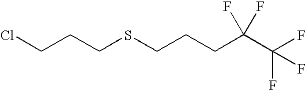

Prepared as described for SM2-b using thioacetic acid S-(4,4,5,5,5-pentafluoro-pentyl) ester (15.0 g, 63.5 mmol) and 1-chloro-3-iodopropane (19.5 g, 95.3 mmol) as starting materials. The crude product (17.8 g) was used in the next step.

¹H NMR (CDCl₃) δ 1.90 (m, 2H), 2.04 (m, 2H), 2.18 (m, 2H), 2.61 (t, J=7.0 Hz, 2H), 2.68 (t, J=7.0 Hz, 2H), 3.66 (t, J=6.3 Hz, 2H).

### c. 1-Iodo-3-(4,4,5,5,5-pentafluoro-pentylsulfanyl)-propane

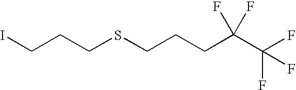

Prepared as described for SM1-b using 1-chloro-3-(4,4,5,5,5-pentafluoro-pentylsulfanyl)-propane (17.8 g, 65.8 mmol) and NaI (14.8 g, 98.6 mmol) as starting materials to give the title compound (20.0 g, 84%).

¹H NMR (CDCl₃) δ 1.90 (m, 2H), 2.07 (m, 2H), 2.18 (m, 2H), 2.61 (t, J=7.2 Hz, 2H), 2.63 (t, J=7.0 Hz, 2H), 3.29 (t, J=6.7 Hz, 2H).

### d. 1-Methylamino-3-(4,4,5,5,5-pentafluoro-pentylsulfanyl)-propane

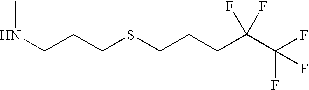

1-Iodo-3-(4,4,5,5,5-pentafluoro-pentylsulfanyl)-propane (20.0 g, 55.2 mmol) was added to a solution of MeNH₂ (90 mL, aq. 40%) and MeCN (400 mL). The solution was stirred at 90° C. over night and was then concentrated at reduced pressure. The residue was partitioned between CH₂Cl₂ and NaHCO₃ (sat.). The aqueous phase was extracted with CH₂Cl₂ and the combined organic phases were dried (Na₂SO₄) and concentrated at reduced pressure to give the title compound (13.0 g, 89%) as an oil.

¹H NMR (CDCl₃) δ 1.77 (m, 2H), 1.89 (m, 2H), 2.17 (m, 2H), 2.44 (s, 3H), 2.58 (t, J=7.3 Hz, 2H), 2.60 (t, J=7.1 Hz, 2H), 2.68 (t, J=7.0 Hz, 2H).

SM4

### 11-(3,17β-Dihydroxy-estra-1,3,5(10)-triene-7(-yl)-undecanoic acid n-butyl-methyl-amide (ICI 164.384)

### a. 3,17β-Di(tetrahydropyranyloxy)-estra-1,3,5(10)-triene

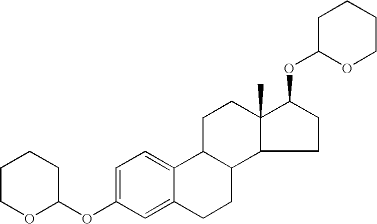

2,3-Dihydropyran (30 ml, 328 mmol) was added to a solution of 3,17β-dihydroxy-estra-1,3,5(10)-triene (20.0 g, 73.5 mmol) and p-TSA (0.2 g) in CH₂Cl₂ (200 ml). The reaction mixture was stirred for 3 h at room temperature. EtN(iPr)₂ (0.5 ml) was added and the reaction mixture was 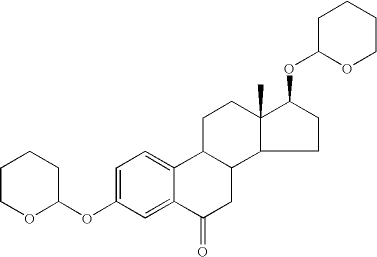 concentrated at reduced pressure and purified on column chromatography (heptane-CH₂Cl₂, 1:1 then CH₂Cl₂) to give the title compound (32.3 g, 100%) as an oil, which crystallized on standing.

R_{f} (heptane-EtOAc, 1:1)=0.79 ¹H NMR (CDCl₃) δ 0.80, 0.82 (2s, 3H), 2.83 (m, 2H), 3.49 (m, 1H), 3.59 (m, 1H), 3.71, 3.72 (2t, J=8 Hz, 1H), 3.92 (m, 2H), 4.65, 4.67 (2m, 1H), 5.38 (broad s, 1H), 6.78 (d, J=2 Hz, 1H), 6.84 (d, J=8,6 Hz, 2 Hz, 1H), 7.18, 7.20 (2d, J=8.6 Hz, 2 Hz, 1H).

### b. 3,17β-Di(tetrahydropyranyloxy)-6-keto-estra-1,3,5(10)-triene

HN(iPr)₂ (17.3 ml, 123 mmol) was added to a solution of n-BuLi (56.0 ml, 2.2 M in hexanes, 123 mmol) in THF (170 ml) under N2 at -20° C. The temperature was lowered to −78° C. and a solution of t-BuOK (13.8 g, 123 mmol) in THF (125 ml) was added. After stirring for 10 min a solution of 3,17β-di(tetrahydropyranyloxy)-estra-1,3,5(10)-triene (13.6 g, 30.9 mmol) in THF (70 ml) was added dropwise under 15 min. The reaction mixture was stirred at −78° C. for 3 h. B(OMe)₃ (45.0 ml, 396 mmol) was added dropwise and the reaction mixture was then stirred at 0° C. for 1.5 h. H₂O₂ (85 ml, aq 30%) was added to give first a turbid reaction mixture then a white precipitated gum (borates, mechanical stirrer or big magnetic stirring bar recommended). After stirring for 1 h at room temperature, the reaction mixture was cooled to 0° C. and aq. Na₂S₂O₃ (100 ml, 1.0 M) was added in portions. After stirring for 20 min the reaction mixture was partitioned between EtOAc and water. The organic phase was washed with water and brine, dried (Na₂SO₄) and concentrated at reduced pressure to give the 6-hydroxy derivative (14.8 g, quant., R_{f} (heptane-EtOAc, 1:1)=0.58, contained 15-20% starting material by NMR).

The 6-hydroxy derivative (14.7 g) was dissolved in CH₂Cl₂ (150 ml) and pyridinium chlorochromate (PCC, 14.7 g, 68 mmol) was added at 0° C. under N₂ in portions for 15 min. The reaction mixture was stirred at 0° C. for 15 min, then at room temperature for 1.5 h. Et₂O (150 ml) was added and after 5 min stirring, the slurry was filtered through silica. The filtrate was concentrated at reduced pressure and purified on column chromatography (heptane-EtOAc, 5:1) to give the title compound (7.50 g, 51%) as a syrup.

R_{f} (heptane-EtOAc, 3:1)=0.38 ¹H NMR (CDCl₃) δ 0.81, 0.82 (2s, 3H), 2.20 (m, 1H), 2.35 (m, 1H), 2.47 (m, 1H), 2.73 (dd, J=16.9, 3.4 Hz, 1H), 3.50 (m, 1H), 3.60 (m, 1H), 3.72, 3.75 (2t, J=8.5 Hz, 1H), 3.90 (m, 2H), 4.64, 4.68 (2m, 1H), 5.47 (m, 1H), 7.22 (m, 1H), 7.34 (m, 1H), 7.71,7.72 (2d, J=2.7 Hz, 1H).

### c. 11-(3,17β-Di(tetrahydropyranyloxy)-6-keto-estra-1,3,5(10)-triene-7α-yl)-undecanoic acid n-butyl-methyl-amide

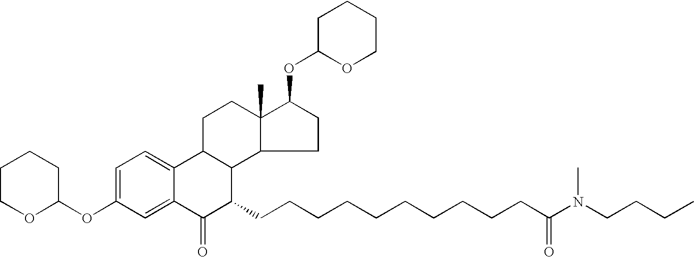

t-BuOK (2.04 g, 18.2 mmol) was added to a solution of 3,17β-di(tetrahydropyranyloxy)-6-keto-estra-1,3,5(10)-triene (7.50 g, 16.5 mmol) in dimethoxyethane (75 ml) under N₂. After 10 min stirring BEt₃ (20.0 ml, 1.0 M in THF, 20.0 mmol) was added and the reaction mixture was stirred for 1 h. A solution of 11-iodo-undecanoic acid n-butyl-methyl-amide (6.48 g, 17.0 mmol) in dimethoxyethane (10 ml) was added. The reaction mixture was stirred for 1 h and then a second batch of t-BuOK (2.04 g, 18.2 mmol) was added. The reaction mixture was stirred over night and was then partitioned between Et₂O and water. The organic phase was washed with water and brine, dried (Na₂SO₄) and concentrated at reduced pressure. The residue was purified on column chromatography (heptane-EtOAc, 3:1 then 2:1) to give the title compound (6.87 g, 59%) as an oil.

R_{f} (heptane-EtOAc, 2:1)=0.29 ¹H NMR (CDCl₃) δ 0.80, 0.82 (2s, 3H), 0.92, 0.95 (2t, J=7.2 Hz, 3H), 2.28 (m, 2H), 2.35 (m, 1H), 2.44 (m, 1H), 2.70 (m, 1H), 2.90, 2.96 (2s, 3H), 3.25, 3.26 (2t, J=7.5 Hz, 2H), 3.49 (m, 1H), 3.61 (m, 1H), 3.74, 3.77 (2t, J=8.5 Hz, 1H), 3.91 (m, 2H), 4.65, 4.68 (m, 1H), 5.46 (m, 1H), 7.20 (d, J=8.6 Hz, 1H), 7.31,7.32 (2d, J=8.6, 1H), 7.69 (broad s, 1H).

### d. 11-(3,17β-Dihydroxy-estra-1,3,5(10)-triene-7α-yl)-undecanoic acid n-butyl-methyl-amide (ICI 164.384)

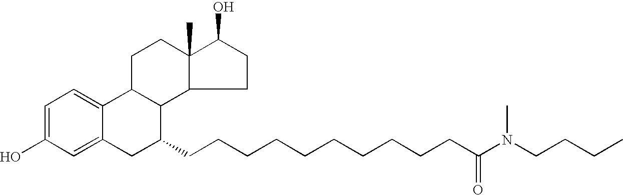

BF₃.OEt₂ (195 ml) was added dropwise to a solution of 11-(3,17β-di(tetrahydropyranyloxy)-6-keto-estra-1,3,5(10)-triene-7α-yl)-undecanoic acid n-butyl-methyl-amide (6.87 g, 9.70 mmol) and HSiEt₃ (97 ml) in CH₂Cl₂ (500 ml) at 0° C. under N₂. The reaction mixture was stirred over night at room temperature and was then slowly poored into aq. K₂CO₃ (1000 ml, 1.0 M) at 0° C. Et₂O (500 ml) was added and after stirring for 30 min the organic phase was washed with water and brine, dried (Na₂SO₄) and concentrated at reduced pressure. The residue was purified on column chromatography (heptane-EtOAc, 1:1) to give the title compound (3.91 g, 77%) as an oil.

R_{f} (heptane-EtOAc, 1:1)=0.21 ¹H NMR (CDCl₃) δ 0.78 (s, 3H), 0.92, 0.95 (2t, J=7.3 Hz, 3H), 1.90 (bd, J=12 Hz, 1H), 2.07-2.18 (m, 1H), 2.25-2.30 (m, 4H), 2.76 (d, J=16.8, 1H), 2.85 (dd, J=16.8, 5.0 Hz, 1H), 2.93, 2.98 (2s, 3H), 3.26 (t, J=7.5 Hz, 1H), 3.38 (m, 1H), 3.75 (broad t, J=7.5 Hz, 1H), 6.41,6.47 (2 bs, 1H), 6.59 (d, J=2.6 Hz, 1H), 6.65 (dd, J=8.5, 2.6 Hz, 1H), 7.13 (d, J=8.5 Hz, 1H).

SM5

### 3,17β-Dihydroxy-7α-[9-(4,4,5,5,5-pentafluoro-n-pentyl)thiononyl]-estra-1,3,5(10)-triene

### a. 3,17β-Di(tetrahydropyranyloxy-6-keto-7α-[9-(4,4,5,5,5-pentafluoro-n-pentyl)thiononyl]-estra-1,3,5(10)-triene

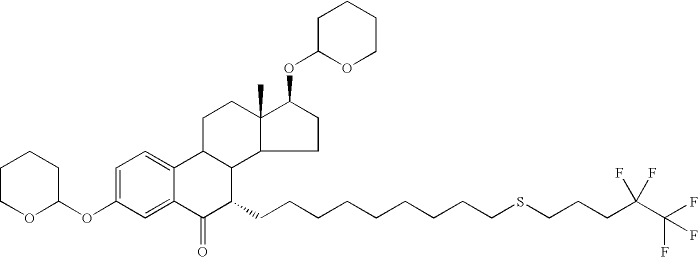

Prepared as described for SM4-c using 3,17β-di(tet-rahydropyranyloxy)-6-keto-estra-1,3,5(10)-triene (4.79 g, 10.5 mmol) and 1-iodo-9-(4,4,5,5,5-pentafluoro-pentyl-sulfanyl)-nonane (4.91 g, 11.0 mmol) as starting materials. The crude product was purified on column chromatography (heptane-EtOAc, 10:1) to give the title compound (3.8 g, 49%) as an oil.

R_{f} (heptane-EtOAc, 1:1)=0.77 ¹H NMR (CDCl₃) δ 0.80, 0.82 (2s, 3H), 2.35 (m, 1H), 2.44 (m, 1H), 2.49 (t, J=7.4 Hz, 2H), 2.58 (t, J=7.0 Hz, 2H), 2.70 (m, 1H), 3.50 (m, 1H), 3.61 (m, 1H), 3.74, 3.77 (2t, J=8 Hz, 1H), 3.90 (m, 2H), 4.65, 4.68 (2m, 1H), 5.46 (m, 1H), 7.20 (d, J=8.6 Hz, 1H), 7.31,7.32 (2d, J=8.6 Hz, 1H), 7.69 (broad s, 1H).

### b. 3,17β-Dihydroxy-7α-[9-(4,4,5,5,5-pentafluoro-n-pentyl)thiononyl]-estra-1,3,5(10)-triene

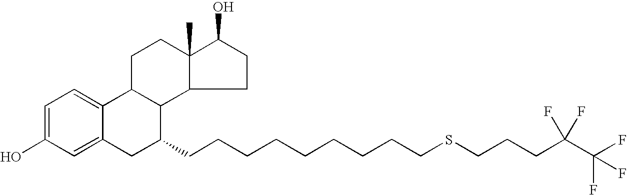

Prepared as described for SM4-d using 3,17β-di(tetrahydropyranyloxy-6-keto-7α-[9-(4,4,5,5,5-pentafluoro-n-pentyl)thiononyl]-estra-1,3,5(10)-triene (3.67 g, 4.75 mmol) as starting material. The crude product was purified on column chromatography (heptane-EtOAc, 2:1) to give the title compound (1.97 g, 70%) as an oil.

R_{f} (heptane-EtOAc, 2:1)=0.32 ¹H NMR (CDCl₃) δ 0.78 (s, 3H), 1.73 (m, 1H), 1.84-1.94 (m, 3H), 2.07-2.24 (m, 3H), 2.25-2.34 (m, 2H), 2.50 (t, J=7.4 Hz, 2H), 2.58 (t, J=7.0 Hz, 2H), 2.71 (d, J=16.8 Hz, 1H), 2.86 (dd, J=16.8, 5.0 Hz, 1H), 3.75 (t, J=8.5 Hz, 1H), 4.68 (broad s, 1H), 6.54 (d, J=2.6 Hz, 1H), 6.62 (dd, J=8.4, 2.6 Hz, 1H), 7.15 (d, J=8.4 Hz, 1H).

SM6

### 16α-(Dimethylthexyl)-silanyloxy-17-(1,2-ethylene)-3-tetrahydropyranyloxy-estra-1,3,5(10)-triene)

### a. 3-Hydroxy-17-methylene-estra-1,3,5(10)-triene

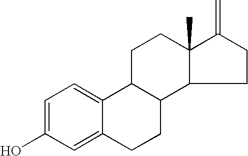

t-BuOK (31.4 g, 280 mmol) was added to a slurry of Ph₃PCH₃Br (100 g, 280 mmol) in dry toluene (350 ml) under N₂. The temperature was raised to 100° C. and the solution was stirred for 30 min. Estrone (25.0 g, 92.5 mmol) was then added in portions and the reaction mixture was stirred for 30 min. After cooling, acetone (30 ml) was added, the reaction mixture was stirred for 20 min and was then filtered through silica gel. The residue was purified on column chromatography (heptane-EtOAc, 3:1) to give the title compound (24.1 g, 97%) as white crystals.

R_{f} (heptane-EtOAc, 2:1)=0.55 ¹H NMR (CDCl₃) δ 0.83 (s, 3H), 1.26 (m, 1H), 1.33-1.61 (m, 5H), 1.82 (m, 1H), 1.90-2.00 (m, 2H), 2.21 (td, J=11,4 Hz, 1H), 2.25-2.40 (m, 2H), 2.55 (m, 1H), 2.78-2.92 (m, 2H), 4.54 (s, 1H), 4.69 (m, 2H), 6.57 (d, J=2.7 Hz, 1H), 6.64 (dd, J=8.4, 2.7 Hz, 1H), 7.18 (d, J=8.4 Hz, 1H).

### b. 3,16α-Dihydroxy-17-methylene-estra-1,3,5(10)-triene 3-O-benzoate

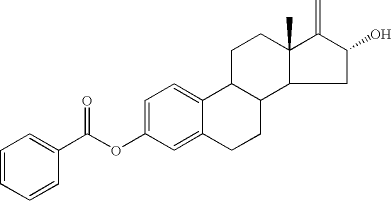

A solution of 3-hydroxy-17-methylene-estra-1,3,5(10)-triene (21.8 g, 81.2 mmol), SeO₂ (300 mg, 2.70 mmol) and t-butylhydroperoxide (150 ml, 150 mmol, 1.0 M in toluene) was stirred over night. The product precipitated from the solution. Heptane (150 ml) was added and the slurry was stirred for 5 min. The precipitate (ca 20 g) was collected by filtration and was dissolved in CH₂Cl₂ (500 ml). NaOH (aq., 500 ml, 1.0 M) and benzoylchloride (20.0 ml, 172 mmol) were added and the reaction mixture was vigorously stirred over night. The organic phase was dried (Na₂SO₄), concentrated at reduced pressure and purified on column chromatography (CH₂Cl₂-EtOAc, 20:1) to give the title compound (16.5 g, 52%) as white crystals.

R_{f} (heptane-EtOAc, 1:1)=0.38 ¹H NMR (CDCl₃) δ 0.84 (s, 3H), 1.41-1.67 (m, 6H), 1.80-2.02 (m, 3H), 2.29-2.45 (m, 2H), 2.85-2.98 (m, 2H), 4.72 (broad s, 1H), 4.94 (d, J=2.1 Hz, 1H), 5.09 (d, J=1.7 Hz, 1H), 6.93 (d, J=2.5 Hz, 1H), 6.97 (dd, J=8.5, 2.5 Hz, 1H), 7.34 (d, J=8.5 Hz, 1H), 7.50 (t, J=7.5 Hz, 2H), 7.63 (tt, J=7.5, 1.3 Hz, 1H), 8.20 (dd, J=7.5, 1.3 Hz, 2H).

### c. 17-(1,2-Ethylene)-3,16α-dihydroxy-estra-1,3,5(10)-triene 3-O-benzoate

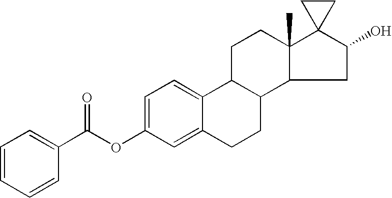

CH₂I₂ (53.6 g, 200 mmol) was added dropwise to a solution of ZnEt₂ (100 ml, 1.0 M in heptane, 100 mmol) in CH₂Cl₂ (250 ml) under N₂ at −10° C. The reaction mixture was stirred for 10 min at −10° C. and then a solution of 3,16α-dihydroxy-17-methylene-estra-1,3,5(10)-triene 3-O-benzoate (19.4 g, 50.0 mmol) in CH₂Cl₂ (125 ml) was slowly added dropwise.

The cooling bath was removed and the reaction mixture was stirred at ambient temperature for 3 h and then partitioned between Et₂O (500 ml) and aq. HCl (400 ml, 0.5 M). The organic phase was washed with water and brine, dried (Na₂SO₄) and concentrated at reduced pressure. The residue was dissolved in EtOAc and precipitated with heptane and collected by filtration to give the title compound (18.6 g, 92%) as yellow crystals.

R_{f} (heptane-EtOAc, 2:1)=0.29 ¹H NMR (CDCl₃) δ 0.42-0.60 (m, 3H), 0.70-0.76 (m, 1H), 0.84 (s, 3H), 2.27-2.36 (m, 2H), 2.85-2.98 (m, 2H), 4.20 (d, J=7.3 Hz, 1H), 6.93 (d, J=2.3 Hz, 1H), 6.97 (dd, J=8.4, 2.3 Hz, 1H), 7.32 (d, J=8.4 Hz, 1H), 7.50 (t, J=7.6 Hz, 2H), 7.63 (t, J=7.6 Hz, 1H), 8.19 (d, J=7.6 Hz, 2H).

### d. 16α-(Dimethylthexyl)-silanyloxy-17-(1,2-ethylene)-3-tetrahydropyranyloxy-estra-1,3,5(10)-triene

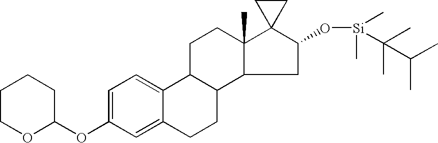

Dimethylthexylchlorosilane (2.75 g, 15.4 mmol) was added to a solution of imidazole (2.19 g, 32.2 mmol) and 17-(1,2-ethylene)-3,16α-dihydroxy-estra-1,3,5(10)-triene 3-O-benzoate (5.18 g, 12.9 mmol) in DMF (10 ml) and CH₂Cl₂ (10 ml). The reaction mixture was stirred over night and was then partitioned between Et₂O and water. The organic phase was washed with aq. HCl (0.5 M), water and brine, dried (Na₂SO₄) and concentrated at reduced pressure to give the crude 16α-O-silylether (7.22 g).

R_{f} (heptane-EtOAc, 10:1)=0.46 ¹H NMR (CDCl₃) δ 0.28-0.39 (m, 2H), 0.45-0.51 (m, 1H), 0.8 (m, 1H), 4.30 (d, J=8.3 Hz, 1H).

The crude 16α-O-silylether (7.22 g) was dissolved in THF (70 ml) and MeOH (30 ml). NaOH (aq., 30 ml, 1.0 M) was added and the reaction mixture was stirred for 1 h. The reaction mixture was partitioned between Et₂O and water. The organic phase was washed with water and brine, dried (Na₂SO₄) and concentrated at reduced pressure. The residue was purified on column chromatography (heptane-EtOAc, 10:1) to give the free phenol (5.88 g) contaminated by ca 4% methylbenzoate.

R_{f} (heptane-EtOAc, 2:1)=0.52 ¹H NMR (CDCl₃) δ 0.01, 0.07 (2s, 6H), 0.32 (m, 2H), 0.46 (m, 1H), 0.77 (m, 1H), 0.82 (s, 3H), 0.82 (s, 6H), 0.87, 0.88 (2d, J=6.9 Hz, 6H), 2.18-2.28 (m, 2H), 2.75-2.88 (m, 2H), 4.29 (d, J=7.9 Hz, 1H), 4.57 (s, 1H), 6.55 (d, J=2.7 Hz, 1H), 6.61 (dd, J=8.4, 2.7 Hz, 1H), 7.13 (d, J=8.4 Hz, 1H).

The free phenol (5.88 g) was dissolved in CH₂Cl₂ (20 ml). 2,3-Dihydropyran (2.0 ml, 21.9 mmol) and p-TSA (20 mg) was added and the reaction mixture was stirred for 30 min. EtN(iPr)₂ (0.1 ml) was added and the reaction mixture was concentrated at reduced pressure. The residue was purified on column chromatography (heptane-EtOAc, 50:1) to give the title compound (6.65 g, 98%) as an oil.

R_{f} (heptane-EtOAc, 10:1)=0.45 ¹H NMR (CDCl₃) δ 0.01, 0.07 (2s, 6H), 0.31 (m, 2H), 0.46 (m, 1H), 0.77 (m, 1H), 0.81 (s, 3H), 0.82 (s, 6H), 0.86, 0.88 (2s, 6H), 2.24 (m, 2H), 2.4 (m, 2H), 3.58 (m, 1H), 3.92 (m, 1H), 4.29 (d, J=8.0 Hz, 1H), 5.38 (s, 1H), 6.78 (s, 1H), 6.83 (d, J=8.6 Hz, 1H), 7.17 (d, J=8.6 Hz, 1H)

### EXAMPLE 1

### 11-(3,16α-Dihydroxy-17-methylene-estra-1,3,5(10)-triene-7α-yl)-undecanoic acid n-butyl-methyl-amide

### a. 11-(3,17β-Dihydroxy-estra-1,3,5(10)-triene-7α-yl)-undecanoic acid n-butyl-methyl-amide 3-O-benzoate

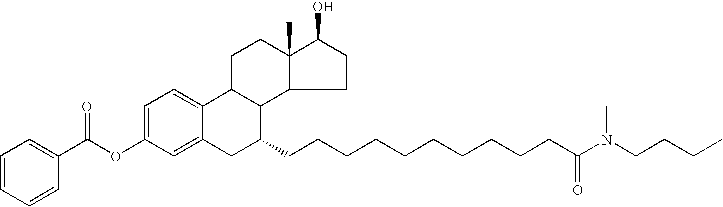

Benzoyl chloride (500 μL, 4.30 mmol) was added to a solution of 11-(3,17β-dihydroxy-estra-1,3,5(10)-triene-7α-yl)-undecanoic acid n-butyl-methyl-amide (1.13 g, 2.15 mmol) in CH₂Cl₂ (20 ml) and NaOH (10 ml, 1.0 M aq.). The reaction mixture was stirred over night and then partitioned between Et₂O and water. The organic phase was washed with water and brine, dried (Na₂SO₄) and concentrated at reduced pressure to give the title compound (1.36 g, quant.) as an oil.

R_{f} (heptane-EtOAc, 1:1)=0.18 ¹H NMR (CDCl₃) δ 0.80 (s, 3H), 0.92, 0.95 (2t, J=7.3 Hz, 3H), 1.77 (m, 1H), 1.93 (m, 1H), 2.14 (m, 1H), 2.28 (m, 2H), 2.33-2.43 (m, 2H), 2.79 (d, J=17.0 Hz, 1H), 2.89-2.98 (m, 1H), 2.90, 2.95 (2s, 3H), 3.24, 3.35 (2t, J=7.5 Hz, 2H), 3.77 (broad t, J=8 Hz, 1H), 6.93 (d, J=2.3 Hz, 1H), 6.98 (dd, J=8.4, 2.3 Hz, 1H), 7.34 (d, J=8.4 Hz, 1H), 7.51 (t, J=8, 2H), 7.63 (t, J=8, 1H), 8.19 (d, J=8, 2H).

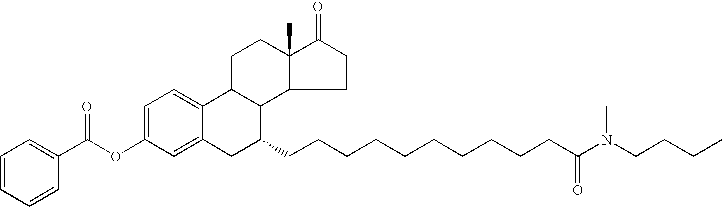

### b. 11-(3-Hydroxy-17-keto-estra-1,3,5(10)-triene-7α-yl)-undecanoic acid n-butyl-methyl-amide 3-O-benzoate

Pyridinium chlorochromate (PCC, 1.00 g, 4.64 mmol) was added in portions to a solution of 11-(3,17β-dihydroxy-estra-1,3,5(10)-triene-7α-yl)-undecanoic acid n-butyl-methyl-amide 3-O-benzoate (1.36 g, 2.16 mmol) in CH₂Cl₂ (15.0 ml) at 0° C. under N₂. The cooling bath was removed and the reaction mixture was stirred at room temperature for 3 h. Et₂O (100 ml) was added and after 10 min stirring, the slurry was purified on column chromatography (Et₂O) to give the title compound (1.22 g, 90%) as an oil.

R_{f} (heptane-EtOAc, 1:1)=0.36 ¹H NMR (CDCl₃) δ 0.92, 0.95 (2t, J=7.4 Hz, 3H), 0.92 (s, 3H), 1.81 (dt, J=2.4, 11 Hz, 1H), 1.87-2.02 (m, 3H), 2.18 (dt, J=19, 8.5 Hz, 1H), 2.28 (m, 2H), 2.40-2.51 (m, 3H), 2.85 (d, J=16.9 Hz, 1H), 2.90, 2.95 (2s, 3H), 2.94-3.02 (m, 1H), 3.24, 3.35 (2t, J=7.5 Hz, 2H), 6.95 (d, J=2.3 Hz, 1H), 7.00 (dd, J=8.5, 2.3 Hz, 1H), 7.34 (d, J=8.5 Hz, 1H), 7.51 (t, J=7.5, 2H), 7.63 (t, J=7.5, 1H), 8.19 (d, J=7.5, 2H).

### c. 11-(3-Hydroxy-17-methylene-estra-1,3,5(10)-triene-7α(-yl)-undecanoic acid n-butyl-methyl-amide

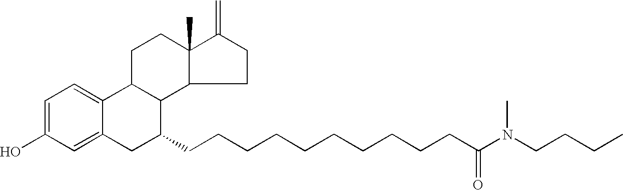

t-BuOK (112 mg, 1.00 mmol) was added to a solution of Ph₃PCH₃Br (357 mg, 1.00 mmol) in dry DMSO (1.0 ml) under N₂. The temperature was raised to 120° C. and a solution of 11-(3-hydroxy-17-keto-estra-1,3,5(10)-triene-7α-yl)-undecanoic acid n-butyl-methyl-amide 3-O-benzoate (207 mg, 0.330 mmol) in dry DMSO (0.5 ml) was added. The reaction mixture was stirred for 30 min, cooled and partitioned between Et₂O and water. The organic phase was washed with water and brine, dried (Na₂SO₄) and concentrated at reduced pressure. The residue was purified on column chromatography (heptane-EtOAc, 2:1) to give the title compound (157 mg, 76%) as an oil.

R_{f} (heptane-EtOAc, 2:1)=0.20 ¹H NMR (CDCl₃) δ 0.82 (s, 3H), 0.92, 0.95 (2t, J=7.3 Hz, 3H), 1.92 (bd, J=11.9 Hz, 1H), 2.25-2.40 (m, 5H), 2.42-2.59 (m, 1H), 2.71 (d, J=16.7 Hz, 1H), 2.87 (dd, J=16.7, 5.0 Hz, 1H), 2.93, 2.98 (2s, 3H), 3.26 (t, J=7.6 Hz, 1H), 3.38 (m, 1H), 4.67 (broad s, 2H), 6.53, 6.58 (2 broad s, 1H), 6.60 (d, J=22.5 Hz, 1H), 6.66 (dd, J=8.4, 2.5 Hz, 1H), 7.14 (d, J=8.4 Hz, 1H).

### d. 11-(3,16α-Dihydroxy-17-methylene-estra-1,3,5(10)-triene-7α-yl)-undecanoic acid n-butyl-methyl-amide

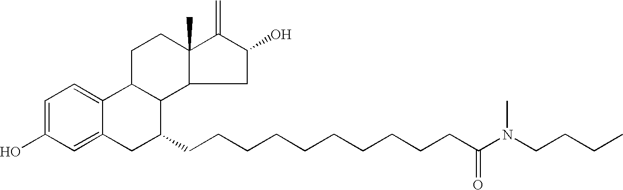

A mixture of 11-(3-hydroxy-17-methylene-estra-1,3,5(10)-triene-7α-yl)-undecanoic acid n-butyl-methyl-amide (232 mg, 0.445 mmol), SeO₂ (15 mg, 0.14 mmol) and t-butyl-hydroperoxide (1.00 ml, 1.00 mmol, 1.0 M in toluene) was stirred for 4 h. The reaction mixture was then partitioned between Et₂O (30 ml) and aq. FeSO₄ (0.5 M, 5 ml). The organic phase was washed with water and brine, dried (Na₂SO₄) and concentrated at reduced pressure. The residue was purified on column chromatography (heptane-EtOAc, 2:1) to give the title compound (127 mg, 53%) as an oil.

R_{f} (heptane-EtOAc, 1:1)=0.38 ¹H NMR (CDCl₃) δ 0.83 (s, 3H), 0.92, 0.95 (2t, J=7.3 Hz, 3H), 2.27-2.42 (m, 4H), 2.72 (d, J=16.7 Hz, 1H), 2.86 (dd, J=16.7, 5.0 Hz, 1H), 2.93, 2.98 (2s, 3H), 3.26 (t, J=7.6 Hz, 1H), 3.38 (m, 1H), 4.72 (broad t, 1H), 4.91 (d, J=2.0 Hz, 1H), 5.08 (d, J=1.5 Hz, 1H), 6.61 (d, J=2.6 Hz, 1H), 6.66 (dd, J=8.3, 2.6 Hz, 1H), 6.71,6.75 (2 bs, 1H), 7.13 (d, J=8.3 Hz, 1H)

### EXAMPLE 2

### 11-(3,16α-Dihydroxy-17-methylene-estra-1,3,5(10)-triene-7α-yl)-undecanoic acid n-butyl-methyl-amide 3-O-benzoate

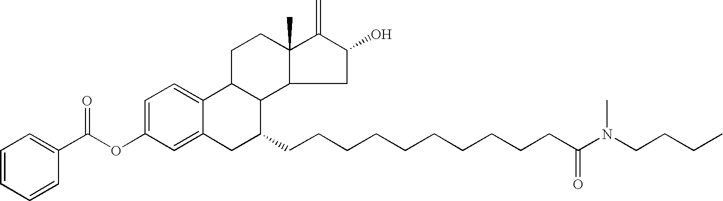

Benzoyl chloride (100 μL, 0.861 mmol) was added to a solution of 11-(3,16α-dihydroxy-17-methylene-estra-1,3,5(10)-triene-7α-yl)-undecanoic acid n-butyl-methyl-amide (106 mg, 0.20 mmol) in CH₂Cl₂ (1.0 ml) and NaOH (1.0 ml, 1.0 M aq.). The reaction mixture was stirred for 9 h and then patitioned between Et₂O and water. The organic phase was dried (Na₂SO₄) and concentrated at reduced pressure. The residue was purified on column chromatography (heptane-EtOAc, 1:1) to give the title compound (124 mg, 98%) as an oil.

R_{f} (heptane-EtOAc, 1:1)=0.42 ¹H NMR (CDCl₃) δ 0.84 (s, 3H), 0.92, 0.95 (2t, J=7.3 Hz, 3H), 2.28 (m, 2H), 2.40-2.52 (m, 2H), 2.81 (d, J=16.7 Hz, 1H), 2.90, 2.96 (2s, 3H), 2.95 (dd, J=16.7, 5.7 Hz, 1H), 3.24, 3.35 (2t, J=7.6 Hz, 2H), 4.74 (broad d, J=6.6 Hz, 1H), 4.93 (d, J=1.9 Hz, 1H), 5.10 (d, J=1.5 Hz, 1H), 6.93 (d, J=2.3 Hz, 1H), 6.99 (dd, J=8.5, 2.3 Hz, 1H), 7.35 (d, J=8.5 Hz, 1H), 7.50 (t, J=7.4 Hz, 2H), 7.63 (t, J=7.4 Hz, 1H), 8.19 (d, J=7.4 Hz, 2H).

### EXAMPLE 3

### 11-(17-(1,2-Ethylene)-3,16α-dihydroxy-estra-1,3,5(10)-triene-7α-yl)-undecanoic acid n-butyl-methyl-amide 3-O-benzoate

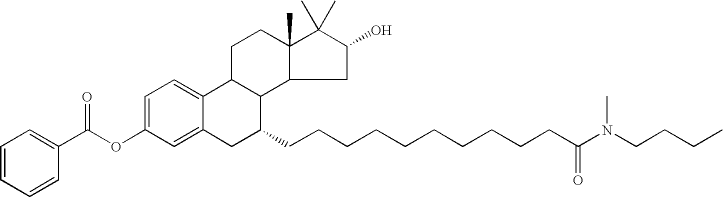

ZnEt₂ (1.0 ml, 1.0 M in heptane,1.0 mmol) was added dropwise to a solution of CH₂I₂ (340 mg, 1.27 mmol) in CH₂Cl₂ (2.5 ml) under N₂ at −10° C. The reaction mixture was stirred for 10 min at −10° C. and then a solution of 11-(3,16α-dihydroxy-17-methylene-estra-1,3,5(10)-triene-7α-yl)-undecanoic acid n-butyl-methyl-amide 3-O-benzoate (124 mg, 0.193 mmol) in CH₂Cl₂ (1.0 ml) was added. The cooling bath was removed and the reaction mixture was stirred at ambient temperature for 5 h and then partitioned between Et₂O (10 ml) and aq. HCl (3 ml, 1.0 M). The organic phase was washed with water and brine, dried (Na₂SO₄) and concentrated at reduced pressure. The residue was purified on column chromatography (heptane-EtOAc, 2:1, 1:1) to give the title compound (84 mg, 66%) as an oil.

R_{f} (heptane-EtOAc, 1:1)=0.50 ¹H NMR (CDCl₃) δ 0.46-0.52 (m, 2H), 0.54-0.61 (m, 1H), 0.73-0.79 (m, 1H), 0.84 (s, 3H), 0.92, 0.95 (2t, J=7.3 Hz, 3H), 2.24-2.37 (m, 3H), 2.41-2.50 (m, 1H), 2.80 (d, J=16.6 Hz, 1H), 2.90, 2.95 (2s, 3H), 2.91-2.98 (m, 1H), 3.24, 3.35 (2t, J=7.5 Hz, 2H), 4.22 (broad s, 1H), 6.93 (d, J=2 Hz, 1H), 6.97 (dd, J=8.6, 2 Hz, 1H), 7.32 (d, J=8.6 Hz, 1H), 7.50 (t, J=7.4 Hz, 2H), 7.63 (t, J=7.4 Hz, 1H), 8.19 (d, J=7.4 Hz, 2H).

### EXAMPLE 4

### 11-(17-(1,2-Ethylene)-3,16α-dihydroxy-estra-1,3,5(10)-triene-7α-yl)-undecanoic acid n-butyl-methyl-amide

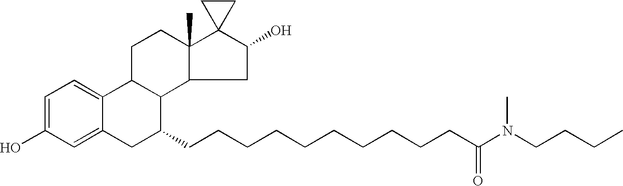

LiOH (1.0 ml, 1.0 M in 50% aq. MeOH,1.0 mmol) was added to a solution of 11-(17-(1,2-ethylene)-3,16α-dihydroxy-estra-1,3,5(10)-triene-7α-yl)-undecanoic acid n-butyl-methyl-amide 3-O-benzoate (84 mg, 0.128 mmol) in THF (2.0 ml). The reaction mixture was stirred for 30 min and was then patitioned between Et₂O (10 ml) and aq. HCl (1.5 ml, 1.0 M) and brine (2 ml). The organic phase was washed with water and brine, dried (Na₂SO₄) and concentrated at reduced pressure. The residue was purified on column chromatography (heptane-EtOAc, 2:1,1:1) to give the title compound (70 mg, 99%) as an oil.

R_{f} (heptane-EtOAc, 1:1)=0.41 ¹H NMR (CDCl₃) δ 0.45-0.51 (m, 2H), 0.53-0.59 (m, 1H), 0.70-0.77 (m, 1H), 0.82 (s, 3H), 0.92, 0.95 (2t, J=7.3 Hz, 3H), 1.82-2.00 (m, 2H), 2.24-2.41 (m, 4H), 2.72 (d, J=16.6 Hz, 1H), 2.86 (dd, J=16.6, 4.9 Hz, 1H), 2.93, 2.98 (2s, 3H), 3.26 (t, J=7.7 Hz, 1H), 3.37 (m, 1H), 4.20 (broad t, J=6 Hz, 1H), 6.36, 6.42 (2s, 1H), 6.60 (d, J=2.3 Hz, 1H), 6.64 (dd, J=8.4, 2.3 Hz, 1H), 7.12 (d, J=8.4 Hz, 1H).

### EXAMPLE 5

### 3,16α-Dihydroxy-17-methylene-7α-[9-[[(4,4,5,5,5-pentafluoro-n-pentyl)sulfinyl]nonyl]-estra-1,3,5(10)-triene

### a. 3,17β-Dihydroxy-7α-[9-(4,4,5,5,5-pentafluoro-n-pentyl)thiononyl]-estra-1,3,5 (10)-triene 3-O-benzoate

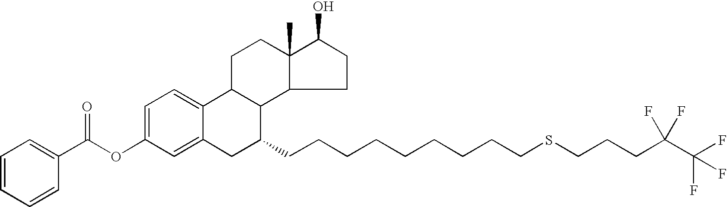

Prepared as described for Example 1-a using 3,17β-dihydroxy-7α-[9-(4,4,5,5,5-pentafluoro-n-pentyl)thiononyl]-estra-1,3,5(10)-triene (250 mg, 0.423 mmol) as starting material to give the title compound (275 mg, 94%) as an oil.

R_{f} (heptane-EtOAc, 2:1)=0.38 ¹H NMR (CDCl₃) δ 0.80 (s, 3H), 1.77 (m, 1H), 1.83-1.97 (m, 3H), 2.09-2.24 (m, 3H), 2.34-2.44 (m, 2H), 2.50 (t, J=7.4 Hz, 2H), 2.58 (t, J=7.0 Hz, 2H), 2.79 (d, J=16.8 Hz, 1H), 2.94 (dd, J=16.8, 4.7 Hz, 1H), 3.76 (t, J=8.5 Hz, 1H), 6.93 (d, J=2.4 Hz, 1H), 6.98 (dd, J=8.4, 2.4 Hz, 1H), 7.34 (d, J=8.4 Hz, 1H), 7.51 (t, J=8 Hz, 2H), 7.63 (t, J=8 Hz, 1H), 8.19 (d, J=8 Hz, 2H).

### b. 3-Hydroxy-17-keto-7α-[9-(4,4,5,5,5-pentafluoro-n-pentyl)thiononyl]-estra-1,3,5(10)-triene 3-O-benzoate

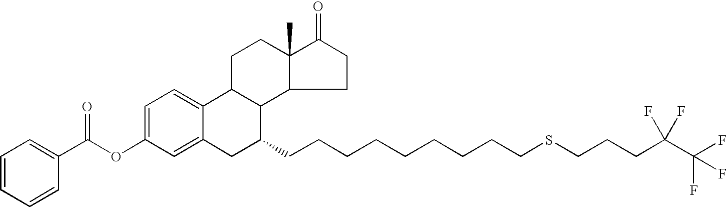

Pyridinium chlorochromate (PCC, 172 mg, 0.800 mmol) was added in portions to a solution of 3,17β-dihydroxy-7α-[9-(4,4,5,5,5-pentafluoro-n-pentyl)thiononyl]-estra-1,3,5(10)-triene 3-O-benzoate (272 mg, 0.391 mmol) in CH₂Cl₂ (2.0 ml) at 0° C. under N₂. The reaction mixture was stirred at 0° C. for 10 min, then at room temperature for 1 h. Et₂O (10 ml) was added and after 5 min stirring, the slurry was purified on column chromatography (Et₂O) to give the title compound (229 mg, 85%) as an oil.

R_{f} (heptane-EtOAc, 2:1)=0.56 ¹H NMR (CDCl₃) δ 0.92 (s, 3H), 2.08-2.24 (m, 3H), 2.40-2.61 (m, 7H), 2.85 (d, J=16.5 Hz, 1H), 2.98 (dd, J=16.5, 5.6 Hz, 1H), 6.95 (d, J=2.2 Hz, 1H), 7.00 (dd, J=8.4, 2.2 Hz, 1H), 7.34 (d, J=8.4 Hz, 1H), 7.51 (t, J=7.5 Hz, 2H), 7.64 (t, J=7.5 Hz, 1H), 8.19 (d, J=7.5 Hz, 2H).

### c. 3-Hydroxy-17-methylene-7α-[9-(4,4,5,5,5-pentafluoro-n-pentyl)thiononyl]-estra-1,3,5(10)-triene

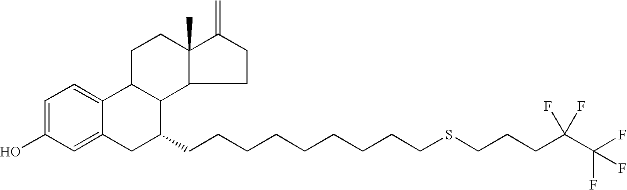

t-BuOK (862 mg, 7.68 mmol) was added to a solution of Ph₃PCH₃Br (2.74 g, 7.68 mmol) in dry DMSO (8.0 ml) under N₂. The temperature was raised to 110° C. during 20 min. This solution was then added portionwise during 5 min to 3-hydroxy-17-keto-7α-[9-(4,4,5,5,5-pentafluoro-n-pentyl)thiononyl]-estra-1,3,5(10)-triene 3-O-benzoate (532 mg, 0.768 mmol) at 110° C. under N₂. The reaction mixture was stirred for another 5 min, cooled and partitioned between Et₂O and water. The organic phase was washed with water acidified with 1M HCl (ca 10 ml) and brine, dried (Na₂SO₄) and concentrated at reduced pressure. The residue was purified on column chromatography (heptane-EtOAc, 10:1) to give the title compound (162 mg, 36%) as an oil.

R_{f} (heptane-EtOAc, 5:1)=0.33 ¹H NMR (CDCl₃) δ 0.82 (s, 3H), 2.17 (m, 2H), 2.50 (t, J=7.4 Hz, 2H), 2.58 (t, J=7.0 Hz, 2H), 2.72 (d, J=16.9 Hz, 1H), 2.88 (dd, J=16.9, 5.3 Hz, 1H), 4.67 (broad s, 2H), 6.55 (d, J=2.6 Hz, 1H), 6.63 (dd, J=8.5, 2.6 Hz, 1H), 7.17 (d, J=8.5 Hz, 1H).

### d. 3,16α-Dihydroxy-17-methylene-7α-[9-[(4,4,5,5,5-pentafluoro-n-pentyl)sulfinyl]nonyl]-estra-1,3,5(10)-triene

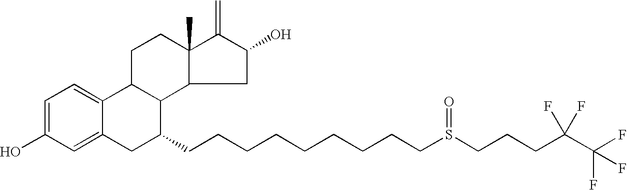

A mixture of 3-hydroxy-17-methylene-7α-[9-(4,4,5,5,5-pentafluoro-n-pentyl)thiononyl]-estra-1,3,5(10)-triene (157 mg, 0.268 mmol), SeO₂ (5 mg, 0.045 mmol) and t-butylhydroperoxide (1.00 ml, 1.00 mmol, 1.0 M in toluene) was stirred for 30 h. The reaction mixture was purified on column chromatography (heptane-EtOAc, 5:1, 3:1, 2:1, 1:2, 1:3) to give the title compound (63 mg, 38%) as an oil.

R_{f} (heptane-EtOAc, 1:3)=0.27 ¹H NMR (CDCl₃) δ 0.83 (s, 3H), 1.94 (broad d, J=8.4 Hz, 1H), 2.10-2.32 (m, 6H), 2.59-2.83 (m, 5H), 2.87 (dd, J=16.8, 5.2 Hz, 1H), 4.72 (broad d, J=6.1 Hz, 1H), 4.92 (d, J=2.0 Hz, 1H), 5.07 (d, J=1.7 Hz, 1H), 5.9, 6.2 (2 broad s, 1H), 6.57 (d, J=2.4 Hz, 1H), 6.64 (m, 1H), 7.14 (d, J=8.3 Hz, 1H).

### EXAMPLE 6

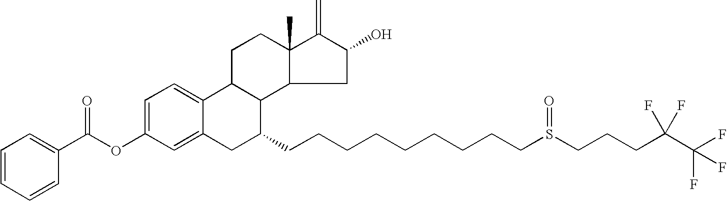

### 3,16α-Dihydroxy-17-methylene-7α-[9-[(4,4,5,5,5-pentafluoro-n-pentyl)sulfinyl]nonyl]-estra-1,3,5(10)-triene 3-O-benzoate

Prepared as described for Example 1-a using 3,16α-dihydroxy-17-methylene-7α-[9-[(4,4,5,5,5-pentafluoro-n-pentyl)sulfinyl]nonyl]-estra-1,3,5 (10)-triene (50 mg, 0.081 mmol) as starting material. The crude product was purified on column chromatography (heptane-EtOAc, 1:1, 1:2) to give the title compound (33 mg, 56%) as an oil.

R_{f} (heptane-EtOAc, 1:3)=0.32 ¹H NMR (CDCl₃) δ 0.84 (s, 3H), 2.10-2.32 (m, 6H), 2.37-2.52 (m, 2H), 2.60-2.77 (m, 4H), 2.80 (d, J=16.4 Hz, 1H), 2.96 (dd, J=16.4, 5.2 Hz, 1H), 4.73 (broad d, J=5.4 Hz, 1H), 4.93 (d, J=1.9 Hz, 1H), 5.09 (d, J=1.4 Hz, 1H), 6.93 (d, J=2.3 Hz, 1H), 6.99 (dd, J=8.6, 2.3 Hz 1H), 7.35 (d, J=8.6 Hz, 1H), 7.51 (t, J=8 Hz, 2H), 7.63 (t, J=8 Hz, 1H), 8.19 (d, J=8 Hz, 2H).

### EXAMPLE 7

### 17-(1,2-Ethylene)-3,16α-dihydroxy-6β-methoxy-7α-[9-(4,4,5,5,5-pentafluoro-n-pentyl)thiononyl]-estra-1,3,5(10)-triene

### a. 16α-(Dimethylthexyl)-silanyloxy-17-(1,2-ethylene)-6-keto-3-tetrahydropyranyloxy-estra-1,3,5(10)-triene

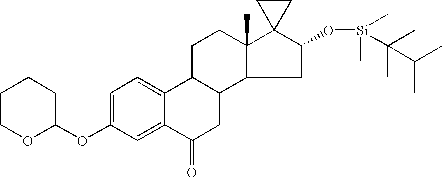

Prepared as described for SM4-c using 16α-(dime-thylthexyl)-silanyloxy-17-(1,2-ethylene)-3-tetrahydro-pyranyloxy-estra-1,3,5(10)-triene (6.62 g, 12.6 mmol) as starting material. The 6-hydroxy derivative (7.01 g, quant., R_{f} (heptane-EtOAc, 5:1)=0.15, contained 20% starting material by NMR). The crude 6-keto product was purified on column chromatography (heptane-EtOAc, 10:1) to give the title compound (4.60 g, 68%) as a syrup.

R_{f} (heptane-EtOAc, 3:1)=0.51 ¹H NMR (CDCl₃) δ 0.01, 0.06 (2s, 6H), 0.35 (m, 2H), 0.48 (m, 1H), 0.80 (m, 1H), 0.82 (s, 3H), 0.82 (s, 6H), 0.87, 0.88 (2d, J=6.8 Hz, 6H), 2.00 (m, 1H), 2.24-2.37 (m, 2H), 2.52 (m, 1H), 2.75 (dd, J=15.8, 2.1 Hz, 1H), 3.60 (m, 1H), 3.88 (m, 1H), 4.28 (d, J=7.8 Hz, 1H), 5.47 (m, 1H), 7.22 (dd, J=8.6, 2.7 Hz, 1H), 7.33 (d, J=8.6 Hz, 1H), 7.72, 7.72 (2d, J=2.7 Hz, 1H).

### b. 16α-(Dimethylthexyl)-silanyloxy-17-(1,2-ethylene)-6-keto-7α-[9-(4,4,5,5,5-pentafluoro-n-pentyl)thiononyl]-3-tetrahydropyranyloxy-estra-1,3,5(10)-triene

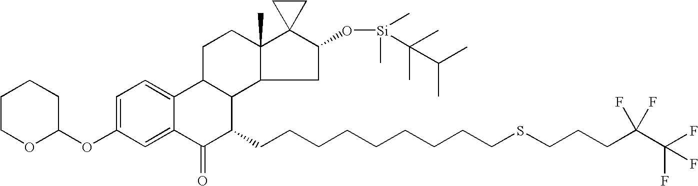

Prepared as described for SM4-c using 16α-(dime-thylthexyl)-silanyloxy-17-(1,2-ethylene)-6-keto-3-tetra-hydropyranyloxy-estra-1,3,5(10)-triene (4.60 g, 8.54 mmol) and 1-iodo-9-(4,4,5,5,5-pentafluoro-pentylsulfanyl)-nonane (4.78 g, 10.7 mmol) as starting materials. The crude product was purified on column chromatography (heptane-EtOAc, 20:1) to give the title compound (4.13 g, 56%) as an oil.

R_{f} (heptane-EtOAc, 10:1)=0.27 ¹H NMR (CDCl₃) δ 0.01, 0.07 (2s, 6H), 0.36 (m, 2H), 0.49 (m, 1H), 0.80 (m, 1H), 0.81 (s, 3H), 0.83 (s, 6H), 0.88 (d, J=6.8 Hz, 6H), 2.17 (m, 2H), 2.34 (m, 1H), 2.44-2.50 (m, 1H), 2.49 (t, J=7.3 Hz, 2H), 2.58 (t, J=7.0 Hz, 2H), 2.75 (td, J=10.4, 3.8 Hz, 1H), 3.61 (m, 1H), 3.91 (m, 1H), 4.23 (d, J=7.4 Hz, 1H), 5.46 (m, 1H), 7.20 (dd, J=8.5, 2.4 Hz, 1H), 7.30 (d, J=8.5 Hz, 1H), 7.69 (d, J=2.4 Hz, 1H).

### c. 16α-(Dimethylthexyl)-silanyloxy-17-(1,2-ethylene)-6α-hydroxy-7α-[9-(4,4,5,5,5-pentafluoro-n-pentyl)thiononyl]-3-tetrahydropyranyloxy-estra-1,3,5(10)-triene

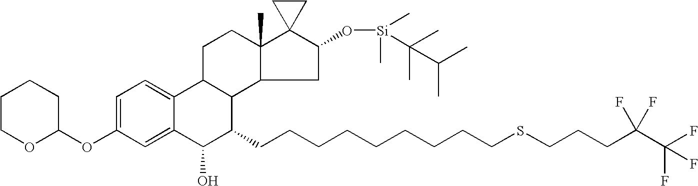

NaBH₄ (285 mg, 7.53 mmol) was added to a solution of 16α-(dimethylthexyl)-silanyloxy-17-(1,2-ethylene)-6-keto-7α-[9-(4,4,5,5,5-pentafluoro-n-pentyl)thiononyl]-3-tetra-hydropyranyloxy-estra-1,3,5(10)-triene (2.85 g, 3.32 mmol) in MeOH (14.0 ml) and THF (7.0 ml). The reaction mixture was stirred over night and was then partitioned between Et₂O and water. The organic phase was washed with water and brine, dried (Na₂SO₄) and concentrated at reduced pressure. The residue was purified on column chromatography (heptane-EtOAc, 10:1,5:1) to give the title compound (2.85 g, quant.) as an oil.

R_{f} (heptane-EtOAc, 5:1)=0.18 ¹H NMR (CDCl₃) δ 0.01, 0.07 (2s, 6H), 0.33 (m, 2H), 0.48 (m, 1H), 0.80 (m, 1H), 0.81 (s, 6H), 0.83 (s, 6H), 0.88, 0.88 (2d, J=6.8 Hz, 6H), 2.09-2.28 (m, 3H), 2.43 (td, J=11, 4 Hz, 1H), 2.49 (t, J=7.3 Hz, 2H), 2.58 (t, J=7.0 Hz, 2H), 3.60 (m, 1H), 3.93 (m, 1H), 4.23 (d, J=7.9 Hz, 1H), 4.88 (m, 1H), 5.40, 5.43 (2t, J=3 Hz, 1H), 6.91 (m, 1H), 7.16 (d, J=8.6 Hz, 1H), 7.33 (d, J=2.5 Hz, 1H).

### d. 16α-(Dimethylthexyl)-silanyloxy-17-(1,2-ethylene)-6β-fluoro-7α-[9-(4,4,5,5,5-pentafluoro-n-pentyl)thiononyl]-3-tetrahydropyranyloxy-estra-1,3,5(10)-triene

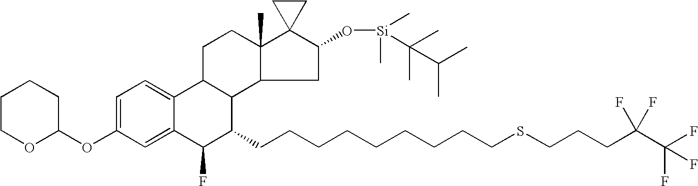

Diethylaminosulfurtrifluoride (DAST, 150 μl, 1.13 mmol) was added to a solution of 16α-(dimethylthexyl)-silanyloxy-17-(1,2-ethylene)-6α-hydroxy-7α-[9-(4,4,5,5,5-pentafluoro-n-pentyl)thiononyl]-3-tetrahydropyranyloxy-estra-1,3,5(10)-triene (780 mg, 0.908 mmol) in CH₂Cl₂ (5.0 ml). The reaction mixture was stirred for 5 min, concentrated at reduced pressure and purified on column chromatography (heptane-EtOAc, 10:1) to give the title compound (629 mg, 80%) as an oil.

R_{f} (heptane-EtOAc, 10:1)=0.41 ¹H NMR (CDCl₃) δ 0.01, 0.07 (2s, 6H), 0.35 (m, 2H), 0.47 (m, 1H), 0.79 (m, 1H), 0.83 (s, 6H), 0.84 (s, 3H), 0.88, 0.88 (2d, J=6.8 Hz, 6H), 2.17 (m, 2H), 2.31 (m, 2H), 2.50 (t, J=7.3 Hz, 2H), 2.58 (t, J=7.0 Hz, 2H), 3.61 (m, 1H), 3.92 (m, 1H), 4.25 (d, J=7.2 Hz, 1H), 5.27, 5.28 (2d, J_{H,F}=51 Hz, 1H), 5.39, 5.42 (2t, J=3.1 Hz, 1H), 7.00-7.09 (m, 2H), 7.25 (d, J=8 Hz, 1H).

### e. 17-(1,2-Ethylene)-3,16α-dihydroxy-6β-methoxy-7α-[9-(4,4,5,5,5-pentafluoro-n-pentyl)thiononyl]-estra-1,3,5(10)-triene

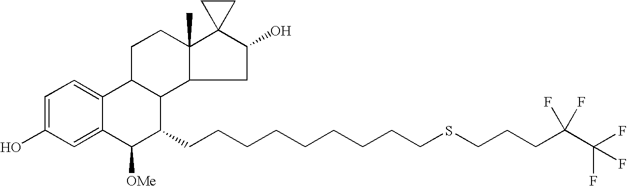

A solution of pyridiniumtosylate in MeOH (0.10 ml, 1.0 M) was added to a solution of 16α-(dimethylthexyl)-silanyloxy-17-(1,2-ethylene)-6β-fluoro-7α-[9-(4,4,5,5,5-pentafluoro-n-pentyl)thiononyl]-3-tetrahydropyranyloxy-estra-1,3,5(10)-triene (248 mg, 0.288 mmol) in MeOH(2.0 ml) and CHCl₃ (2.0 ml). The reaction mixture was stirred for 48 h and was then partitioned between Et₂O and water. The organic phase was washed with water and brine, dried (Na₂SO₄) and concentrated at reduced pressure. The residue was purified on column chromatography (heptane-EtOAc, 3:1, 1:1) to give the title compound (95 mg, 51%).

R_{f} (heptane-EtOAc, 3:1)=0.10 ¹H NMR (CDCl₃) δ 0.46-0.60 (m, 3H), 0.73 (m, 1H), 0.86 (s, 3H), 1.67 (m, 1H), 1.83-2.05 (m, 6H), 2.09-2.32 (m, 4H), 2.50 (t, J=7.4 Hz, 2H), 2.59 (t, J=7.1 Hz, 2H), 3.44 (s, 3H), 3.98 (d, J=1.6 Hz, 1H), 4.23 (t, J=7.2 Hz, 1H), 4.78 (s, 1H), 6.70-6.74 (m, 2H), 7.16 (d, J=8.0 Hz, 1H). MS-ESI [M−H₂O+H]⁺=629

### EXAMPLE 8

### 17-(1,2-Ethylene)-3,16α-dihydroxy-6β-methoxy-7α-[9-[[(4,4,5,5,5-pentafluoro-n-pentyl)sulfinyl]nonyl]-estra-1,3,5(10)-triene

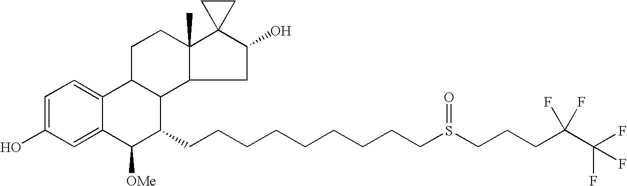

A solution of NaIO₄ in MeOH (0.50 ml, 0.25 mmol, 0.50 M) was added to a solution of 17-(1,2-ethylene)-3,16α-dihydroxy-6β-methoxy-7α-[9-(4,4,5,5,5-pentafluoro-n-pentyl)thiononyl]-estra-1,3,5(10)-triene (79 mg, 0.122 mmol) in MeOH (3.0 ml). The reaction mixture was stirred over night, concentrated at reduced pressure and partitioned between Et₂O and water. The organic phase was washed with water and brine, dried (Na₂SO₄) and concentrated at reduced pressure. The residue was purified on column chromatography (heptane-EtOAc, 1:2, 1:3) to give the title compound (70 mg, 86%).

R_{f} (heptane-EtOAc, 1:3)=0.20 ¹H NMR (CDCl₃) δ 0.45-0.59 (m, 3H), 0.73 (m, 1H), 0.85 (s, 3H), 2.11-2.32 (m, 6H), 2.59-2.84 (m, 4H), 3.42 (s, 3H), 3.98 (s, 1H), 4.22 (broad t, J=7 Hz, 1H), 6.31, 6.51 (2s, 1H), 6.73 (m, 2H), 7.15 (m, 1H). MS-ESI [M−H₂O+H]⁺=645

### EXAMPLE 9

### 17-(1,2-Ethylene)-3,16α-dihydroxy-6-keto-7α-[9-(4,4,5,5,5-pentafluoro-n-pentyl)thiononyl]-estra-1,3,5(10)-triene

### a. 16α-(Dimethylthexyl)-silanyloxy-17-(1,2-ethylene)-3-hydroxy-6-keto-7α-[9-(4,4,5,5,5-pentafluoro-n-pentyl)-thiononyl]-estra-1,3,5(10)-triene

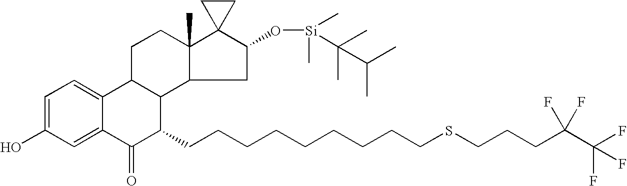

A solution of pyridiniumtosylate in MeOH (0.10 ml, 1.0 M) was added to a solution of 16α-(dimethylthexyl)-silanyloxy-17-(1,2-ethylene)-6-keto-7α-[9-(4,4,5,5,5-pentafluoro-n-pentyl)thiononyl]-3-tetrahydropyranyloxy-estra-1,3,5(10)-triene (160 mg, 0.187 mmol) in MeOH (2.0 ml) and THF (0.5 ml). The reaction mixture was stirred over night, concentrated at reduced pressure and purified on column chromatography (heptane-EtOAc, 10:1, 5:1) to give the title compound (100 mg, 69%).

R_{f} (heptane-EtOAc, 3:1)=0.38 ¹H NMR (CDCl₃) δ 0.01, 0.07 (2s, 6H), 0.37 (m, 2H), 0.49 (m, 1H), 0.80 (m, 1H), 0.81 (s, 3H), 0.83 (s, 6H), 0.89 (d, J=6.9 Hz, 6H), 1.97-2.24 (m, 4H), 2.33 (m, 1H), 2.45-2.50 (m, 1H), 2.49 (t, J=7.5 Hz, 2H), 2.58 (t, J=7.0 Hz, 2H), 2.74 (td, J=11, 4 Hz, 1H), 4.24 (d, J=7.9 Hz, 1H), 5.61 (broad s, 1H), 7.05 (dd, J=8.6, 2.8 Hz, 1H), 7.28 (d, J=8.6 Hz, 1H), 7.56 (d, J=2.8 Hz, 1H).

### b. 17-(1,2-Ethylene)-3,16α-dihydroxy-6-keto-7α-[9-(4,4,5,5,5-pentafluoro-n-pentyl)thiononyl]-estra-1,3,5(10)-triene

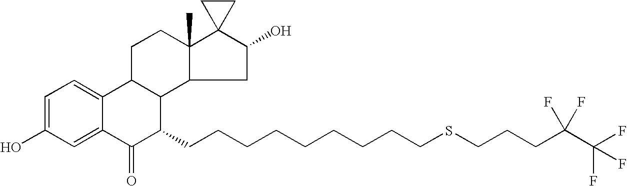

16α-(Dimethylthexyl)-silanyloxy-17-(1,2-ethylene)-3-hydroxy-6-keto-7α-[9-(4,4,5,5,5-pentafluoro-n-pentyl)-thiononyl]-estra-1,3,5(10)-triene (100 mg, 0.129 mmol) was dissolved in a solution of tetrabutylammoniumfluoride trihydrate in THF (0.5 ml, 1.0 M). The reaction mixture was stirred over night at 50° C. and was then partitioned between Et₂O and water. The organic phase was washed with water and brine, dried (Na₂SO₄) and concentrated at reduced pressure. The residue was purified on column chromatography (heptane-EtOAc, 3:1) to give the title compound (70 mg, 86%).

R_{f} (heptane-EtOAc, 2:1)=0.35 ¹H NMR (CDCl₃) δ 0.47-0.62 (m, 3H), 0.78 (m, 1H), 0.82 (s, 3H), 2.02-2.24 (m, 4H), 2.35 (m, 1H), 2.46-2.52 (m, 1H), 2.49 (t, J=7.4 Hz, 2H), 2.58 (t, J=7.0 Hz, 2H), 2.76 (m, 1H), 4.24 (t, J=6.7 Hz, 1H), 6.40 (s, 1H), 7.06 (dd, J=8.5, 2.9 Hz, 1H), 7.28 (d, J=8.5 Hz, 1H), 7.61 (d, J=2.9 Hz, 1H). MS-ESI [M−H₂O+H]⁺=613

### EXAMPLE 10

### 17-(1,2-Ethylene)-3,16α-dihydroxy-6-keto-7α-[9-[(4,4,5,5,5-pentafluoro-n-pentyl)sulfinyl]nonyl]-estra-1,3,5(10)-triene

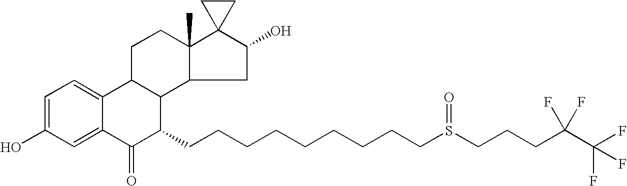

Prepared as described for Example 8 using 17-(1,2-ethylene)-3,16α-dihydroxy-6-keto-7α-[9-(4,4,5,5,5-pentafluoro-n-pentyl)thiononyl]-estra-1,3,5(10)-triene (65 mg, 0.103 mmol) as starting material. The crude product was purified on column chromatography (heptane-EtOAc, 1:2, 1:3) to give the title compound (,46 mg, 69%).

R_{f} (heptane-EtOAc, 1:3)=0.23 ¹H NMR (CDCl₃) δ 0.47-0.61 (m, 3H), 0.77 (m, 1H), 0.82 (s, 3H), 2.47 (broad d, J=1l Hz, 1H), 2.62-2.93 (m, 5H), 4.23 (broad t, J=7 Hz, 1H), 7.03 (m, 1H), 7.25 (d, J=8 Hz, 1H), 7.47-7.55 (m, 2H). MS-ESI [M−H₂O+H]⁺=629

### EXAMPLE 11

### 17-(1,2-Ethylene)-3,6α,16α-trihydroxy-7α-[9-(4,4,5,5,5-pentafluoro-n-pentyl)thiononyl]-estra-1,3,5(10)-triene

### a. 1α-(Dimethylthexyl)-silanyloxy-17-(1,2-ethylene)-3,6α-dihydroxy-7α-[9-(4,4,5,5,5-pentafluoro-n-pentyl)thiononyl]-estra-1,3,5(10)-triene

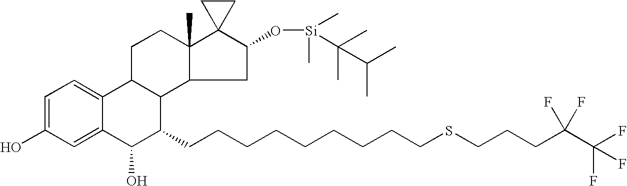

NaBH₄ (20 mg, 0.53 mmol) was added to a solution of 16α-(dimethylthexyl)-silanyloxy-17-(1,2-ethylene)-6-keto-7α-[9-(4,4,5,5,5-pentafluoro-n-pentyl)thiononyl]-3-tetra-hydropyranyloxy-estra-1,3,5(10)-triene (181 mg, 0.211 mmol) in MeOH (1.0 ml) and THF (0.5 ml). The reaction mixture was stirred for 30 min. A solution of pyridiniumtosylate in MeOH (1.0 M, 3.0 ml) was added and the reaction mixture was stirred over night and was then partitioned between Et₂O and water. The organic phase was washed with water and brine, dried (Na₂SO₄) and concentrated at reduced pressure. The residue was purified on column chromatography (heptane-EtOAc, 2:1) to give the title compound (114 mg, 70%).

R_{f} (heptane-EtOAc, 3:1)=0.25 ¹H NMR (CDCl₃) δ 0.01, 0.07 (2s, 6H), 0.34 (m, 2H), 0.47 (m, 1H), 0.80 (m, 1H), 0.82 (s, 3H), 0.83 (s, 6H), 0.88, 0.88 (2d, J=6.9 Hz, 6H), 1.79 (d, J=8.2 Hz, 1H), 1.81-1.96 (m, 4H), 1.99 (m, 1H), 2.09-2.26 (m, 3H), 2.41 (td, J=11, 4 Hz, 1H), 2.49 (t, J=7.4 Hz, 2H), 2.58 (t, J=7.0 Hz, 2H), 4.23 (d, J=7.9 Hz, 1H), 4.87 (s, 1H), 4.88 (m, 1H), 6.70 (dd, J=8.4, 2.8 Hz, lH), 7.07 (d, J=8.4 Hz, 1H), 7.14 (d, J=2.8 Hz, 1H).

### b. 17-(1,2-Ethylene)-3,6α,16α-trihydroxy-7α-[9-(4,4,5,5,5-pentafluoro-n-pentyl)thiononyl]-estra-1,3,5(10)-triene

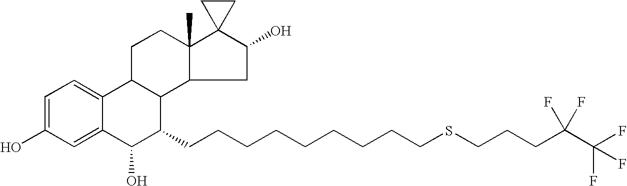

Prepared as described for Example 9-b using 16α-(di-methylthexyl)-silanyloxy-17-(1,2-ethylene)-3,6α-dihydroxy-7α-[9-(4,4,5,5,5-pentafluoro-n-pentyl)thiononyl]-estra-1,3,5(10)-triene (94 mg, 0.121 mmol) as starting material. The crude product was purified on column chromatography (heptane-EtOAc, 2:1) to give the title compound (62 mg, 81%).

R_{f} (heptane-EtOAc, 2:1)=0.22 ¹H NMR (CDCl₃) δ 0.47-0.60 (m, 3H), 0.74 (m, 1H), 0.83 (s, 3H), 1.63 (td, J=11, 2 Hz, 1H), 1.71 (m, 1H), 1.79 (d, J=8.0 Hz, 1H), 1.83-2.04 (m, 4H), 2.09-2.28 (m, 3H), 2.42 (td, J=11, 4 Hz, 1H), 2.49 (t, J=7.4 Hz, 2H), 2.58 (t, J=7.0 Hz, 2H), 4.22 (t, J=7.3 Hz, 1H), 4.87 (s, 1H), 4.90 (broad t, J=6.4 Hz, 1H), 6.71 (dd, J=8.3, 2.7 Hz, 1H), 7.2 (d, J=8.3 Hz, 1H), 7.14 (d, J=2.7 Hz, 1H). MS-ESI [M−H₂O+H]⁺=615

### EXAMPLE 12

### 17-(1,2-Ethylene)-3,6α,16α-trihydroxy-7α-[9-[(4,4,5,5,5-pentafluoro-n-pentyl)sulfinyl]nonyl]-1,3,5(10)-triene

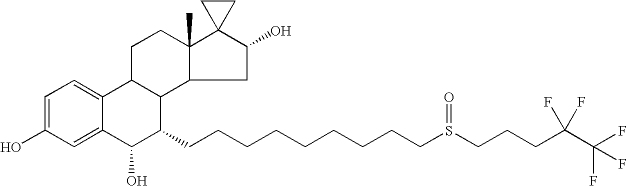

Prepared as described for Example 8 using 17-(1,2-ethylene)-3,6α,16α-trihydroxy-7α-[9-(4,4,5,5,5-penta-fluoro-n-pentyl)thiononyl]-estra-1,3,5(10)-triene (54 mg, 0.085 mmol) as starting material. The crude product was purified on column chromatography (heptane-EtOAc, 1:3, 1:5) to give the title compound (56 mg, quant.).

R_{f} (heptane-EtOAc, 1:3)=0.15 ¹H NMR (CDCl₃) δ 0.44-0.59 (m, 3H), 0.75 (m, 1H), 0.83 (s, 3H), 2.41 (broad t, J=11.5 Hz, 1H), 2.60-2.83 (m, 4H), 4.21 (broad s, 1H), 4.89 (broad t, J=6 Hz, 1H), 6.48, 6.56 (2s, 1H), 6.70 (dd, J=8.5, 2.3 Hz, 1H), 7.10 (d, J=8.5 Hz, 1H), 7.16 (d, J=2.3 Hz, 1H). MS-ESI [M−H₂O+H]⁺=631

### EXAMPLE 13

### 17-(1,2-Ethylene)-3,16α-dihydroxy-6α-methoxy-7α-[9-(4,4,5,5,5-pentafluoro-n-pentyl)thiononyl]-estra-1,3,5(10)-triene

### a. 16α-(Dimethylthexyl)-silanyloxy-17-(1,2-ethylene)-6α-methoxy-7α-[9-(4,4,5,5,5-pentafluoro-n-pentyl)thiononyl]-3-tetrahydropyranyloxy-estra-1,3,5(10)-triene

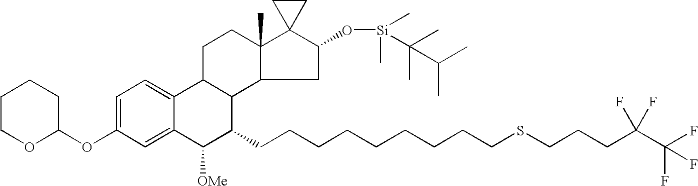

NaH (20 mg, 0.62 mmol) was added to a solution of 16α-(dimethylthexyl)-silanyloxy-17-(1,2-ethylene)-6α-hydroxy-7α-[9-(4,4,5,5,5-pentafluoro-n-pentyl)thiononyl]-3-tetrahydropyranyloxy-estra-1,3,5(10)-triene (232 mg, 0.270 mmol) in THF (2.0 ml) under N₂. MeI (0.150 ml, 2.41 mmol) was added and the reaction mixture was stirred for 4 h, diluted with Et₂O and then filtered through silica gel. The filtrate was concentrated at reduced pressure to give the title compound (205 mg, 87%).

R_{f} (heptane-EtOAc, 3:1)=0.61 ¹H NMR (CDCl₃) δ 0.01, 0.09 (2s, 6H), 0.34 (m, 2H), 0.48 (m, 1H), 0.80 (m, 1H), 0.82 (s, 3H), 0.83 (s, 6H), 0.89, 0.89 (2d, J=6.8 Hz, 6H), 2.45 (td, J=11, 4 Hz, 1H), 2.49 (t, J=7.5 Hz, 2H), 2.58 (t, J=7.0 Hz, 2H), 3.56, 3.56 (2s, 3H), 3.59 (m, 1H), 3.93 (m, 1H), 4.25 (d, J=6.7 Hz, 1H), 4.35 (m, 1H), 5.36, 5.50 (2t, 3 Hz, 1H), 6.89, 6.93 (2dd, J=8.6, 2.8 Hz, 1H), 7.14 (d, J=8.6 Hz, 1H), 7.28 (s, 1H).

### b. 16α-(Dimethylthexyl)-silanyloxy-17-(1,2-ethylene)-3-hydroxy-6α-methoxy-7α-[9-(4,4,5,5,5-pentafluoro-n-pentyl)thiononyl]-estra-1,3,5(10)-triene

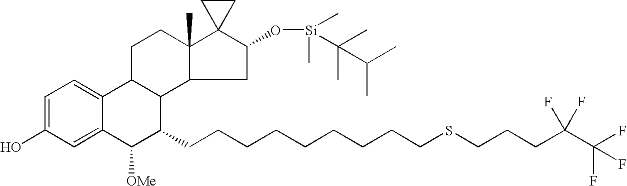

Pyridiniumtosylate (15 mg) was added to a solution of 16α-(dimethylthexyl)-silanyloxy-17-(1,2-ethylene)-6α-methoxy-7α-[9-(4,4,5,5,5-pentafluoro-n-pentyl)thiononyl]-3-tetrahydropyranyloxy-estra-1,3,5(10)-triene (205 mg, 0.235 mmol) in EtOH (2.0 ml). The reaction mixture was stirred over night, concentrated at reduced pressure, redissolved in Et₂O and then filtered through silica gel. The filtrate was concentrated at reduced pressure to give the title compound (178 mg, 96%).

R_{f} (heptane-EtOAc, 3:1)=0.49 ¹H NMR (CDCl₃) δ 0.01, 0.08 (2s, 6H), 0.34 (m, 2H), 0.48 (m, 1H), 0.80 (m, 1H), 0.82 (s, 3H), 0.83 (s, 6H), 0.88, 0.89 (2d, J=6.8 Hz, 6H), 2.09-2.26 (m, 4H), 2.43 (broad t, J=12 Hz, 1H), 2.49 (t, J=7.4 Hz, 2H), 2.58 (t, J=7.0 Hz, 2H), 3.57 (s, 3H), 4.25 (d, J=7.5 Hz, 1H), 4.34 (d, J=4.5 Hz, 1H), 4.64 (s, 1H), 6.68 (dd, J=8.7, 2.7 Hz, 1H), 7.07 (d, J=2.7 Hz, 1H), 7.11 (d, J=8.7 Hz, 1H).

### c. 17-(1,2-Ethylene)-3,16α-dihydroxy-6α-methoxy-7α-[9-(4,4,5,5,5-pentafluoro-n-pentyl)thiononyl]-estra-1,3,5(10)-triene

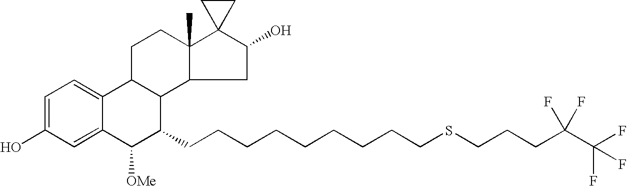

Prepared as described for Example 9-b using 16α-(dimethylthexyl)-silanyloxy-17-(1,2-ethylene)-3-hydroxy-6α-methoxy-7α-[9-(4,4,5,5,5-pentafluoro-n-pentyl)thiononyl]-estra-1,3,5(10)-triene (178 mg, 0.226 mmol) as starting material. The crude product was purified on column chromatography (heptane-EtOAc, 5:1, 3:1) to give the title compound (118 mg, 81%).

R_{f} (heptane-EtOAc, 3:1)=0.29 ¹H NMR (CDCl₃) δ 0.47-0.60 (m, 3H), 0.74 (m, 1H), 0.83 (s, 3H), 2.09-2.28 (m, 4H), 2.43 (td, J=11.0, 3.8 Hz, 1H), 2.49 (t, J=7.4 Hz, 2H), 2.58 (t, J=7.0 Hz, 2H), 3.57 (s, 3H), 4.22 (t, J=7.4 Hz, 1H), 4.36 (d, J=5.0 Hz, 1H), 4.72 (s, 1H), 6.68 (dd, J=8.4, 2.6 Hz, 1H), 7.08 (d, J=2.6 Hz, 1H), 7.11 (d, J=8.4 Hz, 1H). MS-ESI [M−H₂O+H]⁺=629

### EXAMPLE 14

### 17-(1,2-Ethylene)-3,16α-dihydroxy-6α-methoxy-7α-[9-[(4,4,5,5,5-pentafluoro-n-pentyl)sulfinyl]nonyl]-estra-1,3,5(10)-triene

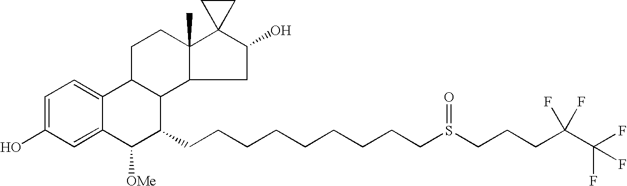

Prepared as described for Example 8 using 17-(1,2-ethylene)-3,16α-dihydroxy-6α-methoxy-7α-[9-(4,4,5,5,5-pentafluoro-n-pentyl)thiononyl]-estra-1,3,5(10)-triene (110 mg, 0.170 mmol) as starting material. The crude product was purified on column chromatography (heptane-EtOAc, 1:2) to give the title compound (94 mg, 83%).

R_{f} (heptane-EtOAc, 1:2)=0.27 ¹H NMR (CDCl₃) δ 0.46-0.60 (m, 3H), 0.74 (m, 1H), 0.83 (s, 3H), 1.87-2.04 (m, 2H), 2.11-2.32 (m, 6H), 2.42 (broad t, J=12 Hz, 1H), 2.60-2.83 (m, 4H), 3.55 (s, 3H), 4.21 (t, J=7.5 Hz, 1H), 4.36 (broad s, 1H), 5.62, 5.87 (2s, 1H), 6.68 (broad d, J=8.5, 1H), 7.10 (m, 2H). MS-ESI [M−H₂O+H]⁺=645

### EXAMPLE 15

### 17-(1,2-Ethylene)-6β-fluoro-3,16α-dihydroxy-7α-[9-(4,4,5,5,5-pentafluoro-n-pentyl)thiononyl]-estra-1,3,5(10)-triene

### a. 16α-(Dimethylthexyl)-silanyloxy-17-(1,2-ethylene)-6β-fluoro-3-hydroxy-7α-[9-(4,4,5,5,5-pentafluoro-n-pentyl)-thiononyl]-estra-1,3,5 (10)-triene

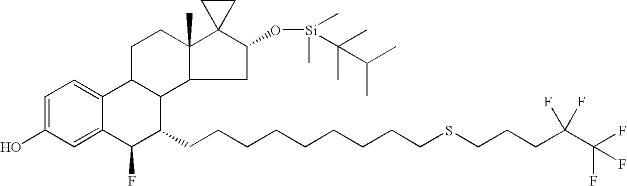

A solution of 16α-(dimethylthexyl)-silanyloxy-17-(1,2-ethylene)-6β-fluoro-7α-[9-(4,4,5,5,5-pentafluoro-n-pentyl)thiononyl]-3-tetrahydropyranyloxy-estra-1,3,5(10)-triene (380 mg, 0.441 mmol) in THF (10 ml) and H₂SO₄ (aq. 1.0 M, 1.0 ml) was stirred for 5 h and was then partitioned between Et₂O and NaHCO₃ (aq.sat.). The organic phase was washed with brine, dried (Na₂SO₄) and concentrated at reduced pressure to give the crude title compound (390 mg).

R_{f} (heptane-EtOAc, 3:1)=0.42 ¹H NMR (CDCl₃) δ 0.01, 0.07 (2s, 6H), 0.35 (m, 2H), 0.48 (m, 1H), 0.79 (m, 1H), 0.83 (s, 6H), 0.84 (s, 3H), 0.88, 0.88 (2d, J=6.9 Hz, 6H), 2.50 (t, J=7.4 Hz, 2H), 2.60 (t, J=7.0 Hz, 2H), 4.26 (d, J=7.4 Hz, 1H), 4.71 (s, 1H), 5.24 (dd, J_{H,F}=51, 1.8 Hz, 1H), 6.79-6.86 (m, 2H), 7.22 (d, J=8.4 Hz, 1H).

### b. 17-(1,2-Ethylene)-6β-fluoro-3,16α-dihydroxy-7α-[9-(4,4,5,5,5-pentafluoro-n-pentyl)thiononyl]-estra-1,3,5(10)-triene

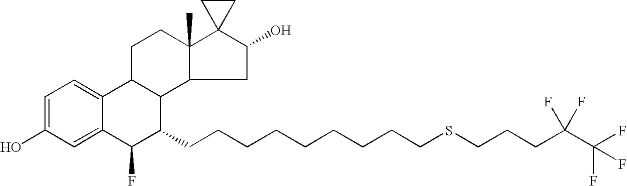

Prepared as described for Example 9-b using 16α-(dimethylthexyl)-silanyloxy-17-(1,2-ethylene)-6β-fluoro-3-hydroxy-7α-[9-(4,4,5,5,5-pentafluoro-n-pentyl)thiononyl]-estra-1,3,5(10)-triene (377 mg) as starting material. The reaction mixture was stirred for 50 h. The crude product was purified on column chromatography (heptane-EtOAc, 5:1, 3:1) to give the title compound (120 mg, 44% in 2 steps).

R_{f} (heptane-EtOAc, 3:1)=0.2 ¹H NMR (CDCl₃) δ 0.47-0.60 (m, 3H), 0.75 (m, 1H), 0.86 (s, 3H), 1.67 (m, 1H), 1.83-2.25 (m, 8H), 2.25-2.38 (m, 2H), 2.50 (t, J=7.4 Hz, 2H), 2.59 (t, J=7.0 Hz, 2H), 4.24 (t, J=6.8 Hz, 1H), 4.82 (s, 1H), 5.26 (dd, J_{H,F}=51, 2 Hz, 1H), 6.80-6.86 (m, 2H), 7.22 (d, J=8.1 Hz, 1H). MS-ESI [M−H₂O+H]⁺=617

### EXAMPLE 16

### 17-(1,2-Ethylene)-6β-fluoro-3,16α-dihydroxy-7α-[9-[(4,4,5,5,5-pentafluoro-n-pentyl)sulfinyl]nonyl]-estra-1,3,5(10)-triene

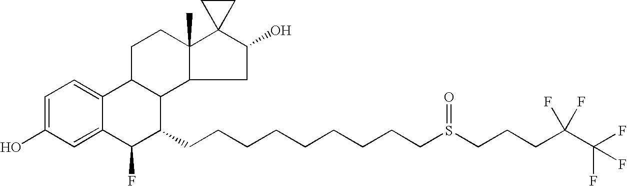

Prepared as described for Example 8 using 17-(1,2-ethylene)-6β-fluoro-3,16α-dihydroxy-7α-[9-(4,4,5,5,5-pentafluoro-n-pentyl)thiononyl]-estra-1,3,5(10)-triene (71 mg, 0.112 mmol) as starting material. The crude product was purified on column chromatography (heptane-EtOAc, 1:2, 1:3) to give the title compound (56 mg, 77%).

R_{f} (heptane-EtOAc, 1:3)=0.28 ¹H NMR (CDCl₃) δ 0.47-0.60 (m, 3H), 0.74 (m, 1H), 0.86 (s, 3H), 2.59-2.85 (m, 4H), 4.23 (t, J=6.7 Hz, 1H), 5.26 (d, J_{H,F}=51 Hz, 1H), 6.32, 6.59 (2s, 1H), 6.81-6.88 (m, 2H), 7.20 (d, J=8.5 Hz, 1H). MS-ESI [M−H₂O+H]⁺=633

### EXAMPLE 17

### 17-(1,2-Ethylene)-3,16α-dihydroxy-7α-[9-(4,4,5,5,5-pentafluoro-n-pentyl)thiononyl]-estra-1,3,5(10)-triene

### a. 6α/β-Chloro-16α-(dimethylthexyl)-silanyloxy-17-(1,2-ethylene)-7α-[9-(4,4,5,5,5-pentafluoro-n-pentyl)thiononyl]-3-tetrahydropyranyloxy-estra-1,3,5(10)-triene

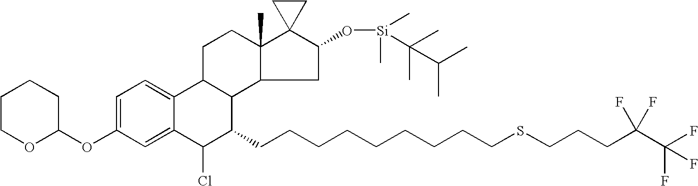

A solution of thionylchloride (59 mg, 0.50 mmol) in CH₂Cl₂ (0.5 ml) was added to a solution of 16α-(dimethylthexyl)-silanyloxy-17-(1,2-ethylene)-6α-hydroxy-7α-[9-(4,4,5,5,5-pentafluoro-n-pentyl)thiononyl]-3-tetrahydropyranyloxy-estra-1,3,5(10)-triene (316 mg, 0.368 mmol) and EtN(iPr)₂ (103 μl, 0.60 mmol) in CH₂Cl₂ (2.0 ml). The reaction mixture was stirred for 30 min and was then partitioned between Et₂O and water. The organic phase was washed with 0.1 M HCl (aq.), water, NaHCO₃ (aq., sat.) and brine, dried (Na₂SO₄) and concentrated at reduced pressure to give the crude title compound (290 mg, 90%).

R_{f} (heptane-EtOAc, 10:1)=0.35 ¹H NMR (CDCl₃) δ 0.01, 0.07 (2s, 6H), 0.34 (m, 2H), 0.47 (m, 1H), 0.79 (m, 1H), 0.81 (s, 3H), 0.82 (s, 6H), 0.88 (d, J=6.8 Hz, 6H), 2.49 (m, 2H), 2.58 (t, J=7.0 Hz, 2H), 3.60 (m, 1H), 3.92 (m, 1H), 4.25 (m, 1H), 5.14 (d, J=8.4 Hz, 1H (6-epimer)), 5.35-5.44 m, 2H (THP, 6-epimer)), 6.90-7.01, 7.13-7.21, 7.41 (3m, 3H).

### b. 16α-(Dimethylthexyl)-silanyloxy-17-(1,2-ethylene)-7α-[9-(4,4,5,5,5-pentafluoro-n-pentyl)thiononyl]-3-tetrahydropyranyloxy-estra-1,3,5(10)-triene

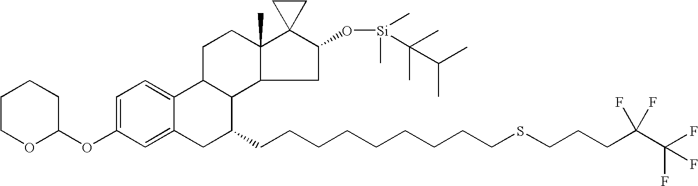

A solution of LiEt₃BH in THF (1.0 ml, 1.0 M) was added to a solution of 6α/β-chloro-16α-(dimethylthexyl)-sila-nyloxy-17-(1,2-ethylene)-7α-[9-(4,4,5,5,5-pentafluoro-n-pentyl)thiononyl]-3-tetrahydropyranyloxy-estra-1,3,5(10)-triene (290 mg, 0.330 mmol) in DME (2.0 ml) under N₂. The temperature was raised to 85° C. and the reaction mixture was stirred for 30 min. Another batch of LiEt₃BH in THF (1.0 ml, 1.0 M) was added and stirring was continued at 85° C. over night. After cooling, the reaction mixture was partitioned between Et₂O and water. The organic phase was washed with water and brine, dried (Na₂SO₄) and concentrated at reduced pressure.

The residue was purified on column chromatography (heptane-EtOAc, 50:1, 20:1) to give the title compound (175 mg, 63%).

R_{f} (heptane-EtOAc, 10:1)=0.39 ¹H NMR (CDCl₃) δ 0.01, 0.07 (2s, 6H), 0.34 (m, 2H), 0.47 (m, 1H), 0.78 (m, 1H), 0.80 (s, 3H), 0.83 (s, 6H), 0.88, 0.88 (2d, J=6.8 Hz, 6H), 2.36 (broad t, J=11.3 Hz, 1H), 2.50 (t, J=7.3 Hz, 2H), 2.58 (t, J=7.0 Hz, 2H), 2.73, 2.74 (2d, J=16.9, 1H), 2.88 (m, 1H), 3.59 (m, 1H), 3.93 (m, 1H), 4.23 (d, J=7.2 Hz, 1H), 5.37 (m, 1H), 6.76 (d, J=2.4 Hz, 1H), 6.83 (m, 1H), 7.17 (d, J=8.5 Hz, 1H).

### c. 16α-(Dimethylthexyl)-silanyloxy-17-(1,2-ethylene)-3-hydroxy-7α-[9-(4,4,5,5,5-pentafluoro-n-pentyl)thiononyl]-estra-1,3,5(10)-triene

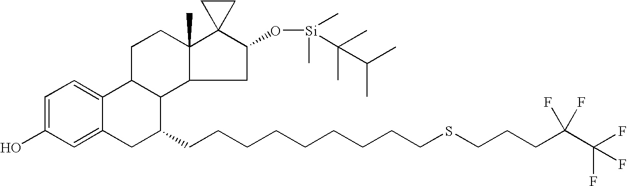

Prepared as described for Example 9-a using 16α-(dimethylthexyl)-silanyloxy-17-(1,2-ethylene)-7α-[9-(4,4,5,5,5-pentafluoro-n-pentyl)thiononyl]-3-tetrahydropyranyloxy-estra-1,3,5(10)-triene (175 mg, 0.208 mmol) as starting material. The crude product was purified on column chromatography (heptane-EtOAc, 10:1, 5:1) to give the title compound (135 mg, 85%).

R_{f} (heptane-EtOAc, 3:1)=0.50 ¹H NMR (CDCl₃) δ 0.01, 0.07 (2s, 6H), 0.34 (m, 2H), 0.48 (m, 1H), 0.79 (m, 1H), 0.81 (s, 3H), 0.83 (s, 6H), 0.88, 0.88 (2d, J=6.8 Hz, 6H), 2.35 (broad t, J=11.4 Hz, 1H), 2.50 (t, J=7.3 Hz, 2H), 2.58 (t, J=7.0 Hz, 2H), 2.71 (d, J=16.7, 1H), 2.86 (dd, J=16.7, 5.2 Hz, 1H), 4.23 (d, J=7.2 Hz, 1H), 4.55 (s, 1H), 6.54 (d, J=2.4 Hz, 1H), 6.60 (dd, J=8.5, 2.4 Hz 1H), 7.14 (d, J=8.5 Hz, 1H).

### d. 17-(1,2-Ethylene)-3,16α-dihydroxy-7α-[9-(4,4,5,5,5-pentafluoro-n-pentyl)thiononyl]-estra-1,3,5(10)-triene

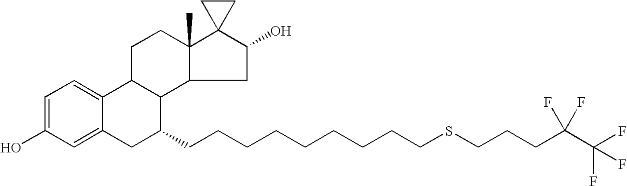

Prepared as described for Example 9-b using 16α-(dimethylthexyl)-silanyloxy-17-(1,2-ethylene)-3-hydroxy-7α-[9-(4,4,5,5,5-pentafluoro-n-pentyl)thiononyl]-estra-1,3,5(10)-triene (85 mg, 0.112 mmol) as starting material. The crude product was purified on column chromatography (heptane-EtOAc, 5:1) to give the title compound (46 mg, 67%).

R_{f} (heptane-EtOAc, 3:1)=0.27 ¹H NMR (CDCl₃) δ 0.47-0.59 (m, 3H), 0.72 (m, 1H), 0.82 (s, 3H), 2.09-2.24 (m, 2H), 2.28 (m, 1H), 2.37 (td, J=11.5, 3.8 Hz, 1H), 2.50 (t, J=7.4 Hz, 2H), 2.58 (t, J=7.0 Hz, 2H), 2.73 (d, J=16.8, 1H), 2.87 (dd, J=16.8, 5.2 Hz, 1H), 4.21 (t, J=6.5 Hz, 1H), 4.61 (s, 1H), 6.54 (d, J=2.6 Hz, 1H), 6.62 (dd, J=8.4, 2.6 Hz, 1H), 7.13 (d, J=8.4 Hz, 1H). MS-ESI [M−H₂O+H]⁺=599

### EXAMPLE 18

### 17-(1,2-Ethylene)-3,16α-dihydroxy-7α-[9-[(4,4,5,5,5-pentafluoro-n-pentyl)sulfinyl]nonyl]-estra-1,3,5(10)-triene

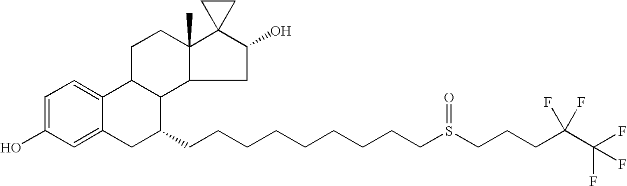

Prepared as described for Example 8 using 17-(1,2-ethylene)-3,16α-dihydroxy-7α-[9-[(4,4,5,5,5-pentafluoro-n-pentyl)sulfinyl]nonyl]-estra-1,3,5(10)-triene (46 mg, 0.075 mmol) as starting material. The crude product was purified on column chromatography (heptane-EtOAc, 1:2) to give the title compound (36 mg, 76%).

R_{f} (heptane-EtOAc, 1:2)=0.25 ¹H NMR (CDCl₃) δ 0.46-0.59 (m, 3H), 0.73 (m, 1H), 0.82 (s, 3H), 1.83-2.00 (m, 2H), 2.12-2.40 (m, 6H), 2.59-2.90 (m, 6H), 4.20 (t, J=6.6 Hz, 1H), 5.95, 6.23 (2s, 1H), 6.56 (d, J=2.4 Hz, 1H), 6.62 (m, 1H), 7.12 (d, J=8.5Hz, 1H). MS-ESI [M−H₂O+H]⁺=615

### EXAMPLE 19

### 17-(1,2-Ethylene)-3,16α-dihydroxy 6-keto-7α-[5-[N-methyl-N-3-(4,4,5,5,5-pentafluoro-n-pentylthio)-propylamino]-pentyl]-estra-1,3,5(10)-triene

### a. 7α-(5-Chloro-n-pentyl)-16α-(dimethylthexyl)-silanyloxy-17-(1,2-ethylene)-6-keto-3-tetrahydropyranyloxy-estra-1,3,5(10)-triene

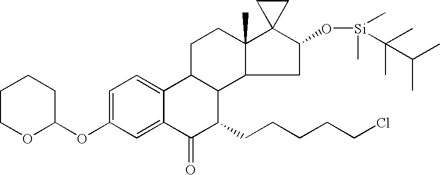

Prepared as described for SM4-c using 16α-(dimethyl-thexyl)-silanyloxy-17-(1,2-ethylene)-6-keto-3-tetrahydropyranyloxy-estra-1,3,5(10)-triene (971 mg, 8.54 mmol) and 1-chloro-5-iodo-pentane (523 mg, 2.25 mmol) as starting materials. The crude product was purified on column chromatography (heptane-EtOAc, 20:1) to give the title compound (511 mg, 44%). R_{f} (heptane-EtOAc, 10:1)=0.26 ¹H NMR (CDCl₃) δ 0.01, 0.07 (2s, 6H), 0.36 (m, 2H), 0.49 (m, 1H), 0.79 (m, 1H), 0.81 (s, 3H), 0.83 (s, 6H), 0.88 (d, J=6.8 Hz, 6H), 2.34 (m, 1H), 2.48 (broad d, J=11.3 Hz, 1H), 2.74 (m, 1H), 3.50 (t, J=6.7 Hz, 2H), 3.61 (m, 1H), 3.90 (m, 1H), 4.23 (d, J=7.8 Hz, 1H), 5.46 (m, 1H), 7.21 (dd, J=8.5 Hz, 1H), 7.31 (d, J=8.5 Hz, 1H), 7.69 (s, 1H).

### b. 16α-(Dimethylthexyl)-silanyloxy-17-(1,2-ethylene)-6-keto-7α-[5-[N-methyl-N-3-(4,4,5,5,5-pentafluoro-n-pentylthio)-propylamino]-pentyl]-3-tetrahydropyranyloxy-estra-1,3,5(10)-triene

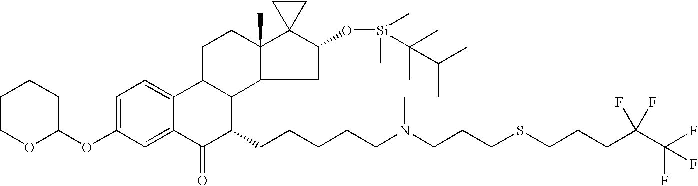

NaI (50 mg, 0.33 mmol) and TBD-methylpolystyrene (350 mg, 0.91 mmol) were added to a solution of 7α-(5-chloro-n-pentyl)-16α-(dimethylthexyl)-silanyloxy-17-(1,2-ethylene)-6-keto-3-tetrahydropyranyloxy-estra-1,3,5(10)-triene (175 mg, 0.272 mmol) and 1-methylamino-3-(4,4,5,5,5-pentafluoro-pentylsulfanyl)-propane (175 mg, 0.660 mmol) in THF (1.0 mL) and MeCN (1.0 mL). The reaction mixture was stirred under microwave-assisted conditions at 180° C. for 1 h. After cooling the reaction mixture was concentrated at reduced pressure and the residue was purified on column chromatography (CHCl₃-MeOH, 40:1, 20:1) to give the title compound (166 mg, 70%) as an oil.

R_{f} (CHCl₃-MeOH, 10:1)=0.50 ¹H NMR (CDCl₃) δ 0.01, 0.06 (2s, 6H), 0.36 (m, 2H), 0.49 (m, 1H), 0.79 (m, 1H), 0.81 (s, 3H), 0.83 (s, 6H), 0.88, 0.89 (2d, J=6.8 Hz, 6H), 2.18 (s, 3H), 2.74 (m, 1H), 3.61 (m, 1H), 3.90 (m, 1H), 4.24 (d, J=7.0 Hz, 1H), 5.46 (m, 1H), 7.20 (d, J=8.6 Hz, 1H), 7.30 (d, J=8.6 Hz, 1H), 7.69 (s, 1H).

### c. 17-(1,2-Ethylene)-16α-hydroxy-6-keto-7α-[5-[N-methyl-N-3-(4,4,5,5,5-pentafluoro-n-pentylthio)-propylamino]-pentyl]-3-tetrahydropyranyloxy-estra-1,3,5(10)-triene

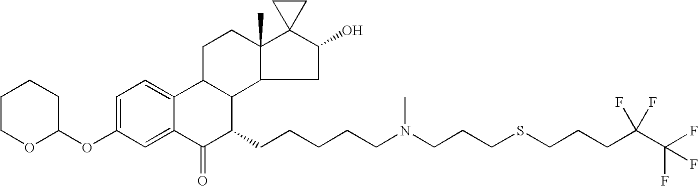

Prepared as described for Example 9-b using 16α-(dimethylthexyl)-silanyloxy-17-(1,2-ethylene)-6-keto-7α-[5-[N-methyl-N-3-(4,4,5,5,5-pentafluoro-n-pentylthio)-propylamino]-pentyl]-3-tetrahydropyranyloxy-estra-1,3,5(10)-triene (179 mg, 0.205 mmol)as starting material. The reaction mixture was stirred under microwave-assisted conditions at 140° C. for 20 min. The crude product was purified on column chromatography (CHCl₃-MeOH, 20:1) to give the title compound (94 mg, 63%) as an oil.

R_{f} (CHCl₃-MeOH, 10:1)=0.40 ¹H NMR (CDCl₃) δ 0.46-0.61 (m, 3H), 0.79 (m, 1H), 0.81 (s, 3H), 2.19 (s, 3H), 2.75 (m, 1H), 3.62 (m, 1H), 3.90 (m, 1H), 4.20 (d, J=7.1 Hz, 1H), 5.47 (m, 1H), 7.21 (dm, J=8.6 Hz, 1H), 7.31 (d, J=8.6 Hz, 1H), 7.69 (m, 1H).

### d. 17-(1,2-Ethylene)-3,16α-dihydroxy-6-keto-7α-[5-[N-methyl-N-3-(4,4,5,5,5-pentafluoro-n-pentylthio)-propylamino]-pentyl]-estra-1,3,5 (10)-triene

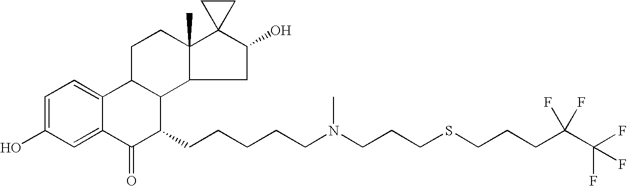

MgCl₂ (19 mg, 0.1 mmol) was added to a solution of 17-(1,2-Ethylene)-16α-hydroxy-6-keto-7α-[5-[N-methyl-N-3-(4,4,5,5,5-pentafluoro-n-pentylthio)-propylamino]-pentyl]-3-tetrahydropyranyloxy-estra-1,3,5(10)-triene (94 mg, 0.129 mmol) in MeOH (2.0 mL). The reaction mixture was stirred under microwave-assisted conditions at 150° C. for 1 h. After cooling the reaction mixture was concentrated at reduced pressure and the residue was partitioned between Et₂O and water. The organic phase was washed with water and brine, dried (Na₂SO₄) and concentrated at reduced pressure. The residue was purified on column chromatography (CHCl₃-MeOH, 20:1) to give the title compound (40 mg, 48%).

R_{f} (CHCl₃-MeOH, 10:1)=0.27 ¹H NMR (CDCl₃) δ 0.46-0.63 (m, 3H), 0.80 (m, 1H), 0.80 (s, 3H), 2.14 (m, 2H), 2.42 (s, 3H), 2.53 (t, J=7.2 Hz, 2H), 2.57 (t, J=7.0 Hz, 2H), 4.19 (d, J=6.9 Hz, 1H), 7.04 (dd, J=8.5, 2.9 Hz, 1H), 7.25 (d, J=8.5 Hz, 1H), 7.41 (d, J=2.9 Hz, 1H). MS-ESI [M+H]⁺=646

### EXAMPLE 20

### 17-(1,2-Ethylene)-3,6α,16α-trihydroxy-7α-[5-[N-methyl-N-3-(4,4,5,5,5-pentafluoro-n-pentylthio)-propylamino]-pentyl]-estra-1,3,5(10)-triene

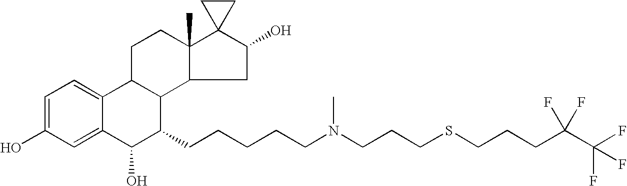

NaBH₄ (50 mg, 1.3 mmol) was added to a solution of 17-(1,2-ethylene)-6-keto-7α-[5-[N-methyl-N-3-(4,4,5,5,5-pentafluoro-n-pentylthio)-propylamino]-pentyl}-estra-1,3,5(10)-triene (29 mg, 0.045 mmol) in MeOH (1.0 ml). The reaction mixture was stirred for 2 h and was then partitioned between Et₂O and water. The organic phase was washed with water and brine, dried (Na₂SO₄) and concentrated at reduced pressure. The residue was purified on column chromatography (CHCl₃-MeOH, 10:1, 5:1) to give the title compound (20 mg, 69%).

R_{f} (CHCl₃-MeOH, 5:1)=0.17 ¹H NMR (CDCl₃) δ 0.44-0.60 (m, 3H), 0.77 (m, 1H), 0.80 (s, 3H), 2.14 (m, 2H), 2.36 (s, 3H), 2.50 (t, J=7.1 Hz, 2H), 2.56 (t, J=7.0 Hz, 2H), 2.63 (m, 2H), 4.19 (d, J=6.7 Hz, 1H) 4.89 (d, J=5.2 Hz, 1H), 6.68 (dd, J=8.5, 2.4 Hz, 1H) 7.07 (d, J=8.5 Hz, 1H), 7.20 (d, J=2.4 Hz, 1H). MS-ESI [M+H]+=648

Biological Models

In Vitro Binding Affinty to the Estrogen Receptor-α (MDS PharmaServices)

Binding affinity was determined in a displacement assay using hER-α (recombinant, insect Sf cells) with 0.5 nM ³H-estradiol as radioligand. The compounds were tested in concentrations from 0.03-10.0 nM. Results are given as IC₅₀ and Ki.

In Vivo Estrogenic Agonism (MDS PharmaServices)

Compounds were administered s.c. (10 mg/kg) for three consecutive days to a group of 5 ICR derived immature female mice weighing approx. 13 g. The animals were sacrificed 24 h after the final dose and wet weight of the uterus was measured. A 50% or greater increase in the uterine weight relative to the vehicle control group indicates possible estrogen agonist activity.

In Vivo Estrogenic Antagonism (MDS PharmaServices)

Compounds were administered s.c. (10 mg/kg) for three consecutive days to a group of 5 ICR derived immature female mice weighing approx. 13 g and challenged with estradiol-benzoate (3 μg/kg s.c.) immediately after each daily dosing. The animals were sacrificed 24 h after the final dose and wet weight of the uterus was measured. A 50% or greater reduction in the estradiol-induced increase in uterine weight indicates possible estrogen antagonist activity.

TABLE 1
Biological effects of representative examples of
the compounds according to the present invention

| | | |
|---|---|---|
| | In vivo | |
| ERα-aff (nM) | agonism | in vivo |

| | Ki | IC₅₀ | (%) | antagonism |
|---|---|---|---|---|
| ICI 164, 384 | 0.76 | 2.67 | 43 | 66 |
| SM4* | | | | |
| ICI 182, 780* | 0.41 | 1.43 | 4 | 66 |
| Ex 1 | 1.00 | 3.50 | 1 | 61 |
| Ex 4 | 0.71 | 2.48 | 4 | 58 |
| Ex 5 | 0.34 | 1.19 | 8 | 55 |
| Ex 8 | 2.91 | 10.2 | | |
| Ex 10 | 1.36 | 4.75 | | |
| Ex 12 | 0.45 | 1.59 | | |
| Ex 14 | >10 | >10 | | |
| Ex 16 | 0.30 | 1.04 | | |
| Ex 18 | 0.26 | 0.92 | | |

*Reference substances.

### REFERENCES

1. Jordan, V. C. J. Med. Chem., Vol. 46, 1081-1111 and 883-908, 2003. Antiestrogens and Selective Estrogen Receptor Modulators as Multifunctional Medicins. 1. Receptor Interactions. and 2. Clinical Considerations and New Agents.

2. Bowler, J. et al. Steroids, Vol. 54, 71-99, 1989. Novel steroidal pure antiestrogens.

3. Brzozowski, A. M. et al. Nature, Vol. 389, 753-8, 1997. Molecular basis of agonism and antagonism in the oestrogen receptor.

4. Pike, A. C. W. et al. Structure, Vol. 9, 145-53, 2001. Structural Insights into the Mode of Action of a Pure Antiestrogen.

5. Tadesco, R. et al. Bioorganic & Biomedicinal Chemistry Letters, Vol. 7, 2919-2924, 1997. 7α,11β-Disubstituted Estrogens: Probes for the Shape of the Ligand Binding Pocket in the Estrogen Receptor. See also refs therein.

6. Tedesco, R. et al. Tetrahedron Letters, Vol. 38, 7997-8000, 1997. An expeditious route to 7α-substituted estradiol derivatives.

1. An anti-estrogenic compound of the general formula I 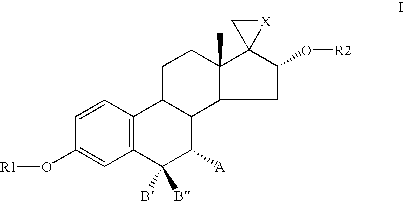
wherein
A is a 8-22 atoms long substituent, wherein substituent A comprises D₁₋₆, wherein D is chosen from the group comprising consisting of R4-C(O)R4, R4S(O)₀₋₂R4, N(R4)₃, R4OR4 and R4(C6H4)R4:
wherein R4 independently represents a bond, of H, or a halogenated or non-halogenated, saturated or unsaturated, mono-, di-, or trivalent C1-C12 hydrocarbon;
B′,B″ are H,H, H,O—R3, O—R3,H, of H,F, or together represent ═O;
R1 is H, or a potentially metabolically unstable group chosen from the group consisting of a straight, branched, or cyclic C1-C6 alkyl, C1-C6 acyl, benzoyl, sulphamoyl, and N-acetyl-sulphamoyl;
R2 is H, or a potentially metabolically unstable group chosen from the group comprising C1-C6 acyl or benzoyl;
R3 is H, C1-C3 alkyl, or a potentially metabolically unstable group chosen from the group comprising C1-C6 acyl, benzoyl, sulphamoyl, or N-acetyl-sulphamoyl; and
X is methylene or a single bond, or
pharmaceutically acceptable salts of the compounds of the general formula I.

2. A compound according to claim 1,
wherein A is
—CH₂)₄₋₆N((CH₂)₀₋₂H)(CH₂)₂₋₄S(O)₀₋₂(CH₂)₂₋₄(CF2)₁₋₃CF₃.

3. A compound according to claim 1,
wherein A is
—CH₂)₇₋₁₁S(O)₀₋₂(CH₂)₂₋₄(CF₂)₁₋₃CF₃.

4. A compound according to claim 1,
wherein A is
—(CH₂)₈₋₁₂C(O)N((CH₂)₀₋₂H)(CY₂)₂₋₆Y
wherein Y is chosen from H or F.

5. A compound according to any of claims 1-4, wherein R1 is hydrogen, methyl, acetyl, benzoyl, sulphamoyl, or N-acetyl-sulphamoyl.

6. A compound according to any of claims 1-4, wherein R3 is H, methyl, or a potentially metabolically unstable group chosen from the group comprising C1-C6 acyl, benzoyl, sulphamoyl, or N-acetyl-sulphamoyl.

7. A compound according to claim 1,
wherein A is
—CH₂)₄₋₆N((CH₂)₀₋₂H)(CH₂)₂₋₄S(O)₀₋₂(CH₂)₂₋₄(CF₂)₁₋₃CF₃,
—CH₂)₇₋₁₁S(O)₀₋₂(CH₂)₂₋₄(CF₂)₁₋₃CF₃,
—CH₂)₈₋₁₂C(O)N((CH₂)₀₋₂H)(CY₂)₂₋₆Y,
wherein Y is chosen from H or F,
—(CH₂)₈₋₉CH(CO₂H)(CH₂)₂₋₅(CF₂)₁₋₃CF₃,
—C₆H₄-p-O(CH₂)₃₋₆S(O)₀₋₂(CH₂)₂₋₄(CF₂)₁₋₃CF₃,
—C₆H₄-p-O(CH₂)₂NMe₂;
R1 is hydrogen, methyl, acetyl, benzoyl, sulphamoyl, or N-acetyl-sulphamoyl;
R2 is hydrogen; and
R3 is H, methyl, or a potentially metabolically unstable group chosen from the group consisting of C1-C6 acyl, benzoyl, sulphamoyl, oand N-acetyl-sulphamoyl.

8. A compound according to claim 1,
wherein A is
—CH₂)₄₋₆N(CH₃)(CH₂)₂₋₄S(O)₀₋₂(CH₂)₂₋₄(CF₂)₁₋₃CF₃,
—CH₂)₇₋₁₁S(O)₀₋₂(CH₂)₂₋₄(CF₂)₁₋₃CF₃,
—(CH₂)₁₀C(O)N(CH₃)(CY₂)₂₋₆Y,
wherein Y is chosen from H or F,
or
—CH₂)₈₋₉CH(CO₂H)(CH₂)₂₋₅(CF₂)₁₋₃CF₃;
B′,B″ are H,H; of H,O—R3; O—R3,H; or H,F;
R1 is H, methyl, acetyl, or sulphamoyl; and
R3 is H, methyl, or acyl;

9. A compound according to claim 1, wherein A is
—(CH₂)₄₋₆N(CH₃)(CH₂)₃S(O)₀₋₂(CH₂)₃CF₃,
—(CH₂)₈₋₁₀S(O)₀₋₂(CH₂)₂₋₄(CF₂)₁₋₃CF₃,
or
—(CH₂)₈₋₉CH(CO₂H)(CH₂)₂₋₅(CF₂)₁₋₃—CF₃
and
R3 is H.

10. A compound according to claim 1 chosen from the group consisting of:
11-(3,16α-Dihydroxy-17-methylene-estra-1,3,5(10)-triene-7α-yl)-undecanoic acid n-butyl-methyl-amide,
11-(3,16α-Dihydroxy-17-methylene-estra-1,3,5(10)-triene-7α-yl)-undecanoic acid n-butyl-methyl-amide 3-O-benzoate,
11-(3,16α-Dihydroxy-17-methylene-estra-1,3,5(10)-triene-7α-yl)-undecanoic acid (2,2,3,3,4,4,4-heptafluoro)-n-butyl-methyl-amide,
3,16α-Dihydroxy-17-methylene-7α-[9-[(4,4,5,5,5-pentafluoro-n-pentyl)thio]nonyl]-estra-1,3,5(10)-triene,
3,16α-Dihydroxy-17-methylene-7α-[9-[(4,4,5,5,5-pentafluoro-n-pentyl)sulfinyl]nonyl]-estra-1,3,5(10)-triene,
3,16α-Dihydroxy-17-methylene-7α-[9-[(4,4,5,5,5-pentafluoro-n-pentyl)sulfinyl]nonyl]-estra-1,3,5(10)-triene 3-0-acetate,
3,16α-Dihydroxy-17-methylene-7α-[9-[(4,4,5,5,5-pentafluoro-n-pentyl)sulfinyl]nonyl]-estra-1,3,5(10)-triene 3-O-sulfamate,
3,16α-Dihydroxy-17-methylene-7α-[9-[(4,4,5,5,5-pentafluoro-n-pentyl)sulfinyl]nonyl]-estra-1,3,5(10)-triene 3-O-benzoate,
3,16α-Dihydroxy-17-methylene-7α-[9-[(4,4,5,5,5-pentafluoro-n-pentyl)sulfonyl]nonyl]-estra-1,3,5(10)-triene,
3,16α-Dihydroxy-17-methylene-7α-[9-[(4,4,5,5,5-pentafluoro-n-pentyl)sulfinyl]octyl]-estra-1,3,5(10)-triene,
7α-[9-[(2,2,3,3,4,4,4-Heptafluoro-n-butyl)sulfinyl]nonyl]-3,16α-dihydroxy-17-methylene-estra-1,3,5(10)-triene,
3,16α-Dihydroxy-17-methylene-7α-[9-[(3,3,4,4,5,5,6,6,6-nonafluoro-n-hexyl)sulfonyl]nonyl]-estra-1,3,5(10)-triene,
3,16α-Dihydroxy-17-methylene-7α-[9-[(4,4,5,5,6,6,7,7,7-nonafluoro-n-heptyl)sulfonyl]nonyl]-estra-1,3,5(10)-triene,
3,16α-Dihydroxy-17-methylene-7α-[5-[N-methyl-N-3-(4,4,5,5,5-pentafluoro-n-pentylthio)-propylamino]-pentyl]-estra-1,3,5(10)-triene,
3,16α-Dihydroxy-17-methylene-7α-[5-[N-methyl-N-3-(4,4,5,5,5-pentafluoro-n-pentylsulfinyl)-propylamino]-pentyl]-estra-1,3,5(10)-triene,
3,16α-Dihydroxy-17-methylene-7α-[5-[N-methyl-N-3-(4,4,5,5,5-pentafluoro-n-pentylsulfinyl)-propylamino]-pentyl]-estra-1,3,5(10)-triene 3-O-sulfamate,
3,16α-Dihydroxy-17-methylene-7α-[5-[N-methyl-N-3-(4,4,5,5,5-pentafluoro-n-pentylsulfinyl)-propylamino]-pentyl]-estra-1,3,5(10)-triene 3-O-benzoate,
3,16α-Dihydroxy-17-methylene-7α-[5-[N-methyl-N-3-(4,4,5,5,5-pentafluoro-n-pentylsulfonyl)-propylamino]-pentyl]-estra-1,3,5(10)-triene,
3,16α-Dihydroxy-17-methylene-7α-[5-[N-methyl-N-3-(3,3,4,4,5,5,6,6,6-nonafluoro-n-hexyl)-propylamino]-pentyl]-estra-1,3,5(10)-triene,
3,16α-Dihydroxy-17-methylene-7α-[5-[N-methyl-N-3-(4,4,5,5,6,6,7,7,7-nonafluoro-n-heptyl)-propylamino]-pentyl]-estra-1,3,5(10)-triene,
11-(3,16α-Dihydroxy-17-methylene-estra-1,3,5(10)-triene-7α-yl)-2-(4,4,5,5,5-pentafluoro-n-pentyl)-undecanoic acid,
11-(3,16α-Dihydroxy-17-methylene-estra-1,3,5(10)-triene-7α-yl)-2-(4,4,5,5,6,6,7,7,7-nonafluoro-n-heptyl)-undecanoic acid,
11-(3,16α-Dihydroxy-17-methylene-estra-1,3,5(10)-triene-7α-yl)-2-(3,3,4,4,5,5,6,6,6-nonafluoro-n-hexyl)-undecanoic acid,
10-(3,16α-Dihydroxy-17-methylene-estra-1,3,5(10)-triene-7α-yl)-2-(3,3,4,4,5,5,6,6,6-nonafluoro-n-hexyl)-decanoic acid,
11-(3,16α-Dihydroxy-17-methylene-estra-1,3,5(10)-triene-7α-yl)-2-(3,3,4,4,5,5,6,6,6-nonafluoro-n-hexyl)-undecanoic acid methylester,
2-[9-(3,16α-Dihydroxy-17-methylene-estra-1,3,5(10)-triene-7α-yl)-nonyl]-2-(3,3,4,4,5,5,6,6,6-nonafluoro-n-hexyl)-malonic acid,
11-(3,6α,16α-Trihydroxy-17-methylene-estra-1,3,5(10)-triene-7α-yl)-undecanoic acid n-butyl-methyl-amide,
3,6α,16α-Trihydroxy-17-methylene-7α-[9-[(4,4,5,5,5-pentafluoro-n-pentyl)thio]nonyl]-estra-1,3,5(10)-triene,
3,6α,16α-Trihydroxy-17-methylene-7α-[9-[(4,4,5,5,5-pentafluoro-n-pentyl)sulfinyl]nonyl]-estra-1,3,5(10)-triene,
3,6α,16α-Trihydroxy-17-methylene-7α-[9-[(4,4,5,5,5-pentafluoro-n-pentyl)sulfinyl]nonyl]-estra-1,3,5(10)-triene 3-O-sulfamate,
3,6α,16α-Trihydroxy-17-methylene-7α-[5-[N-methyl-N-3-(4,4,5,5,5-pentafluoro-n-pentylthio)-propylamino]-pentyl]-estra-1,3,5(10)-triene,
3,6α,16α-Trihydroxy-17-methylene-7α-[5-[N-methyl-N-3-(4,4,5,5,5-pentafluoro-n-pentylthio)-propylamino]-pentyl]-estra-1,3,5(10)-triene 3-O-sulfamate,
3,6α,16α-Trihydroxy-17-methylene-7α-[5-[N-methyl-N-3-(4,4,5,5,5-pentafluoro-n -pentylsulfinyl)-propylamino]-pentyl]-estra-1,3,5(10)-triene,
3,6α,16α-Trihydroxy-17-methylene-7α-[5-[N-methyl-N-3-(3,3,4,4,5,5,6,6,6-nonafluoro-n-hexyl)-propylamino]-pentyl]-estra-1,3,5(10)-triene,
11-(3,6α,16α-Trihydroxy-17-methylene-estra-1,3,5(10)-triene-7α-yl)-2-(3,3,4,4,5,5,6,6,6-nonafluoro-n-hexyl)-undecanoic acid,
10-(3,6α,16α-Trihydroxy-17-methylene-estra-1,3,5(10)-triene-7α-yl)-2-(3,3,4,4,5,5,6,6,6-nonafluoro-n-hexyl)-decanoic acid,
11-(6β-Fluoro-3,16α-dihydroxy-17-methylene-estra-1,3,5(10)-triene-7α-yl)-undecanoic acid n-butyl-methyl-amide,
6β-Fluoro-3,16α-dihydroxy-17-methylene-7α-[9-[(4,4,5,5,5-pentafluoro-n-pentyl)thio]nonyl]-estra-1,3,5(10)-triene,
6β-Fluoro-3,16α-dihydroxy-17-methylene-7α-[9-[(4,4,5,5,5-pentafluoro-n-pentyl)sulfinyl]nonyl]-estra-1,3,5(10)-triene,
6β-Fluoro-3,16α-dihydroxy-17-methylene-7α-[5-[N-methyl-N-3-(4,4,5,5,5-pentafluoro-n-pentylthio)-propylamino]-pentyl]-estra-1,3,5(10)-triene,
6β-Fluoro-3,16α-dihydroxy-17-methylene-7α-[5-[N-methyl-N-3-(4,4,5,5,5-pentafluoro-n -pentylsulfinyl)-propylamino]-pentyl]-estra-1,3,5(10)-triene,
6β-Fluoro-3,16α-dihydroxy-17-methylene-7α-[5-[N-methyl-N-3-(3,3,4,4,5,5,6,6,6-nonafluoro-n-hexyl)-propylamino]-pentyl]-estra-1,3,5(10)-triene,
11-(6β-Fluoro-3,16α-dihydroxy-17-methylene-estra-1,3,5(10)-triene-7α-yl)-2-(3,3,4,4,5,5,6,6,6-nonafluoro-n-hexyl)-undecanoic acid,
10-(6β-Fluoro-3,16α-dihydroxy-17-methylene-estra-1,3,5(10)-triene-7α-yl)-2-(3,3,4,4,5,5,6,6,6-nonafluoro-n-hexyl)-decanoic acid,
3,6β,16α-Trihydroxy-17-methylene-7α-[9-[(4,4,5,5,5-pentafluoro-n-pentyl)sulfinyl]nonyl]-estra-1,3,5(10)-triene,
3,6β,16α-Trihydroxy-17-methylene-7α-[5-[N-methyl-N-3-(4,4,5,5,5-pentafluoro-n-pentylthio)-propylamino]-pentyl]-estra-1,3,5(10)-triene,
3,6β,16α-Trihydroxy-17-methylene-7α-[5-[N-methyl-N-3-(4,4,5,5,5-pentafluoro-n-pentylsulfinyl)-propylamino]-pentyl]-estra-1,3,5(10)-triene,
11-(3,6,β,16α-Trihydroxy-17-methylene-estra-1,3,5(10)-triene-7α-yl)-2-(3,3,4,4,5,5,6,6,6-nonafluoro-n-hexyl)-undecanoic acid,
11-(17-(1,2-Ethylene)-3,16α-dihydroxy-estra-1,3,5(10)-triene-7α-yl)-undecanoic acid n-butyl-methyl-amide,
11-(17-(1,2-Ethylene)-3,16α-dihydroxy-estra-1,3,5(10)-triene-7α-yl)-undecanoic acid n-butyl-methyl-amide 3-O-benzoate,
11-(17-(1,2-Ethylene)-3,16α-dihydroxy-estra-1,3,5(10)-triene-7α-yl)-undecanoic acid (2,2,3,3,4,4,4-heptafluoro)-n-butyl-methyl-amide,
17-(1,2-Ethylene)-3,16α-dihydroxy-7α-[9-[(4,4,5,5,5-pentafluoro-n-pentyl)thio]nonyl]-estra-1,3,5(10)-triene,
17-(1,2-Ethylene)-3,16α-dihydroxy-7α-[9-[(4,4,5,5,5-pentafluoro-n-pentyl)sulfinyl]nonyl]-estra-1,3,5(10)-triene,
17-(1,2-Ethylene)-3,16α-dihydroxy-7α-[9-[(4,4,5,5,5-pentafluoro-n-pentyl)sulfinyl]nonyl]-estra-1,3,5(10)-triene 3-O-acetate,
17-(1,2-Ethylene)-3,16α-dihydroxy-7α-[9-[(4,4,5,5,5-pentafluoro-n-pentyl)sulfinyl]nonyl]-estra-1,3,5(10)-triene 3-O-sulfamate,
17-(1,2-Ethylene)-3,16α-dihydroxy-7α-[9-[(4,4,5,5,5-pentafluoro-n-pentyl)sulfonyl]nonyl]-estra-1,3,5(10)-triene,
17-(1,2-Ethylene)-3,16α-dihydroxy-7α-[9-[(4,4,5,5,5-pentafluoro-n-pentyl)sulfinyl]octyl]-estra-1,3,5(10)-triene,
17-(1,2-Ethylene)-7α-[9-[(2,2,3,3,4,4,4-heptafluoro-n-butyl)sulfinyl]nonyl]-3,16α-dihydroxy-estra-1,3,5(10)-triene,
17-(1,2-Ethylene)-3,16α-dihydroxy-7α-[9[(3,3,4,4,5,5,6,6,6-nonafluoro-n-hexyl)sulfonyl]nonyl]-estra-1,3,5 (10)-triene,
17-(1,2-Ethylene)-3,16α-dihydroxy-7α-[9-[(4,4,5,5,6,6,7,7,7-nonafluoro-n-heptyl)sulfonyl]nonyl]-estra-1,3,5(10)-triene,
17-(1,2-Ethylene)-3,16α-dihydroxy-7α-[5-[N-methyl-N-3-(4,4,5,5,5-pentafluoro-n-pentylthio)-propylamino]-pentyl]-estra-1,3,5(10)-triene,
17-(1,2-Ethylene)-3,16α-dihydroxy-7α-[5-[N-methyl-N-3-(4,4,5,5,5-pentafluoro-n-pentylthio)-propylarnino]-pentyl]-estra-1,3,5(10)-triene 3-O-benzoate,
17-(1,2-Ethylene)-3,16α-dihydroxy-7α-[5-[N-methyl-N-3-(4,4,5,5,5-pentafluoro-n-pentylthio)-propylamino]-pentyl]-estra-1,3,5(10)-triene 3-O-acetate,
17-(1,2-Ethylene)-3,16(-dihydroxy-7α-[5-[N-methyl-N-3-(4,4,5,5,5-pentafluoro-n-pentylthio)-propylamnino]-pentyl]-estra-1,3,5(10)-triene 3-O-sulfamate,
17-(1,2-Ethylene)-3,16α-dihydroxy-7α-[5-[N-methyl-N-3-(4,4,5,5,5-pentafluoro-n-pentylsulfinyl)-propylamino]-pentyl]-estra-1,3,5(10)-triene,
17-(1,2-Ethylene)-3,16α-dihydroxy-7α-[5-[N-methyl-N-3-(4,4,5,5,5-pentafluoro-n-pentylsulfonyl)-propylamino]-pentyl]-estra-1,3,5(10)-triene,
17-(1,2-Ethylene)-3,16α-dihydroxy-7α-[5-[N-methyl-N-3-(3,3,4,4,5,5,6,6,6-nonafluoro-n-hexyl)-propylamino]-pentyl]-estra-1,3,5(10)-triene,
17-(1,2-Ethylene)-3,16α-dihydroxy-7α-[5-[N-methyl-N-3-(4,4,5,5,6,6,7,7,7-nonafluoro-n-heptyl)-propylamino]-pentyl]-estra-1,3,5(10)-triene,
11-(17-(1,2-Ethylene)-3,16α-dihydroxy-estra-1,3,5(10)-triene-7α-yl)-2-(4,4,5,5,5-pentafluoro-n-pentyl)-undecanoic acid,
11-(17-(1,2-Ethylene)-3,16α-dihydroxy-estra-1,3,5(10)-triene-7α-yl)-2-(4,4,5,5,6,6,7,7,7-nonafluoro-n-heptyl)-undecanoic acid,
11-(17-(1,2-Ethylene)-3,16α-dihydroxy-estra-1,3,5(10)-triene-7α-yl)-2-(3,3,4,4,5,5,6,6,6-nonafluoro-n-hexyl)-undecanoic acid,
10-(17-(1,2-Ethylene)-3,16α-dihydroxy-estra-1,3,5(10)-triene-7α-yl)-2-(3,3,4,4,5,5,6,6,6-nonafluoro-n-hexyl)-decanoic acid,
11-(17-(1,2-Ethylene)-3,16α-dihydroxy-estra-1,3,5(10)-triene-7α-yl)-2-(3,3,4,4,5,5,6,6,6-nonafluoro-n-hexyl)-undecanoic acid methylester,
2-[9-(17-(1,2-Ethylene)-3,16α-dihydroxy-estra-1,3,5(10)-triene-7α-yl)-nonyl]-2-(3,3,4,4,5,5,6,6,6-nonafluoro-n-hexyl)-malonic acid,
11-(17-(1,2-Ethylene)-3,6α,16α-trihydroxy-estra-1,3,5(10)-triene-7α-yl)-undecanoic acid n-butyl-methyl-amide,
11-(17-(1,2-Ethylene)-3,6α,16α-trihydroxy-estra-1,3,5(10)-triene-7α-yl)-undecanoic acid (2,2,3,3,4,4,4-heptafluoro)-n-butyl-methyl-amide,
17-(1,2-Ethylene)-3,6α,16α-trihydroxy-7α-[9-[(4,4,5,5,5-pentafluoro-n-pentyl)thio]nonyl]-estra-1,3,5(10)-triene,
17-(1,2-Ethylene)-3,6α,16α-trihydroxy-7α-[9-[(4,4,5,5,5-pentafluoro-n-pentyl)sulfinyl]nonyl]-estra-1,3,5(10)-triene,
17-(1,2-Ethylene)-3,6α,16α-trihydroxy-7α-[9-[(4,4,5,5,5-pentafluoro-n-pentyl)sulfinyl]nonyl]-estra-1,3,5(10)-triene 3-O-sulfamate, 17-(1,2-Ethylene)-3,6α,16α-trihydroxy-7α-[9-[(4,4,5,5,5-pentafluoro-n-pentyl)sulfonyl]nonyl]-estra-1,3,5(10)-triene,
17-(1,2-Ethylene)-7α-[9-[(2,2,3,3,4,4,4-heptafluoro-n-butyl)sulfinyl]nonyl]-3,6α,16α-trihydroxy-estra-1,3,5(10)-triene,
17-(1,2-Ethylene)-3,6α,16α-trihydroxy-7α-[5-[N-methyl-N-3-(4,4,5,5,5-pentafluoro-n-pentylthio)-propylamino]-pentyl]-estra-1,3,5(10)-triene,
17-(1,2-Ethylene)-3,6α,16α-trihydroxy-7α-[5-[N-methyl-N-3-(4,4,5,5,5-pentafluoro-n-pentylthio)-propylamino]-pentyl]-estra-1,3,5(10)-triene 3-O-sulfamate,
17-(1,2-Ethylene)-3,6α,16α-trihydroxy-7α-[5-[N-methyl-N-3-(4,4,5,5,5-pentafluoro-n-pentylsulfinyl)-propylamino]-pentyl]-estra-1,3,5(10)-triene,
17-(1,2-Ethylene)-3,6α,16α-trihydroxy-7α-[5-[N-methyl-N-3-(4,4,5,5,5-pentafluoro-n-pentylsulfonyl)-propylamino]-pentyl]-estra-1,3,5(10)-triene,
11-(17-(1,2-Ethylene)-3,6α,16α-trihydroxy-estra-1,3,5(10)-triene-7α-yl)-2-(4,4,5,5,5pentafluoro-n-pentyl)-undecanoic -acid,
11-(17-(1,2-Ethylene)-3,6α,16α-trihydroxy-estra-1,3,5(10)-triene-7α-yl)-2-(4,4,5,5,6,6,7,7,7-nonafluoro-n-heptyl)-undecanoic acid,
10-(17-(1,2-Ethylene)-3,6α,16α-trihydroxy-estra-1,3,5(10)-triene-7α-yl)-2-(3,3,4,4,5,5,6,6,6-nonafluoro-n-hexyl)-decanoic acid,
11-(17-(1,2-Ethylene)-3,6α,16α-trihydroxy-estra-1,3,5(10)-triene-7α-yl)-2-(4,4,5,5,5-pentafluoro-n-pentyl)-undecanoic acid methylester,
11-(17-(1,2-Ethylene)-3,6α,16α-trihydroxy-estra-1,3,5(10)-triene-7α-yl)-2-(4,4,5,5,6,6,7,7,7-nonafluoro-n-heptyl)-undecanoic acid methylester,
2-[9-(17-(1,2-Ethylene)-3,6α,16α-trihydroxy-estra-1,3,5(10)-triene-7α-yl)-nonyl]-2-(3,3,4,4,5,5,6,6,6-nonafluoro-n-hexyl)-malonic acid,
11-(17-(1,2-Ethylene)-6β-fluoro-3,16α-dihydroxy-estra-1,3,5(10)-triene-7α-yl)-undecanoic acid n-butyl-methyl-amide,
11-(17-(1,2-Ethylene)-6β-fluoro-3,16α-dihydroxy-estra-1,3,5(10)-triene-7α-yl)-undecanoic acid (2,2,3,3,4,4,4-heptafluoro)-n-butyl-methyl-amide,
17-(1,2-Ethylene)-6β-fluoro-3,16α-dihydroxy-7α-[9-[(4,4,5,5,5-pentafluoro-n-pentyl)thio]nonyl]-estra-1,3,5(10)-triene,
17-(1,2-Ethylene)-6β-fluoro-3,16α-dihydroxy-7α-[9-[(4,4,5,5,5-pentafluoro-n-pentyl)sulfinyl]nonyl]-estra-1,3,5(10)-triene,
17-(1,2-Ethylene)-6β-fluoro-3,16α-dihydroxy-7α-[9-[(4,4,5,5,5-pentafluoro-n-pentyl)sulfinyl]nonyl]-estra-1,3,5(10)-triene 3-O-sulfamte,
17-(1,2-Ethylene)-6β-fluoro-3,16α-dihydroxy-7α-[9-[(4,4,5,5,5-pentafluoro-n-pentyl)sulfonyl]nonyl]-estra-1,3,5(10)-triene,
17-(1,2-Ethylene)-6β-fluoro-3,16α-dihydroxy-7α-[5-[N-methyl-N-3-(4,4,5,5,5-pentafluoro-n-pentylthio)-propylamino]-pentyl]-estra-1,3,5(10)-triene,
17-(1,2-Ethylene)-6β-fluoro-3,16α-dihydroxy-7α-[5-[N-methyl-N-3-(4,4,5,5,5-pentafluoro-n-pentylthio)-propylamino]-pentyl]-estra-1,3,5(10)-triene 3-O-sulfamate,
17-(1,2-Ethylene)-6β-fluoro-3,16α-dihydroxy-7α-[5-[N-methyl-N-3-(4,4,5,5,5-pentafluoro-n-pentylsulfinyl)-propylamino]-pentyl]-estra-1,3,5(10)-triene,
17-(1,2-Ethylene)-6β-fluoro-3,16α-dihydroxy-7α-[5-[N-methyl-N-3-(4,4,5,5,5-pentafluoro-n-pentylsulfonyl)-propylamino]-pentyl]-estra-1,3,5(10)-triene,
11-(17-(1,2-Ethylene)-6β-fluoro-3,16α-dihydroxy-estra-1,3,5(10)-triene-7α-yl)-2-(4,4,5,5,5-pentafluoro-n-pentyl)-undecanoic acid,
11-(17-(1,2-Ethylene)-6β-fluoro-3,16α-dihydroxy-estra-1,3,5(10)-triene-7α-yl)-2-(4,4,5,5,6,6,7,7,7-nonafluoro-n-heptyl)-undecanoic acid,
11-(17-(1,2-Ethylene)-6β-fluoro-3,16α-dihydroxy-estra-1,3,5(10)-triene-7α-yl)-2-(4,4,5,5,6,6,7,7,7-nonafluoro-n-heptyl)-undecanoic acid methylester,
17-(1,2-Ethylene)-3,6β,16α-trihydroxy-7α-[9-[(4,4,5,5,5-pentafluoro-n-pentyl)thio]nonyl]-estra-1,3,5(10)-triene,
17-(1,2-Ethylene)-3,6β,16α-trihydroxy-7α-[9-[(4,4,5,5,5-pentafluoro-n-pentyl)sulfinyl]nonyl]-estra-1,3,5(10)-triene,
17-(1,2-Ethylene)-3,6β,16α-trihydroxy-7α-[5-[N-methyl-N-3-(4,4,5,5,5-pentafluoro-n -pentylthio)-propylamino]-pentyl]-estra-1,3,5(10)-triene,
17-(1,2-Ethylene)-3,6β,16α-trihydroxy-7α-[5-[N-methyl-N-3-(4,4,5,5,5-pentafluoro-n -pentylsulfinyl)-propylamino]-pentyl]-estra-1,3,5(10)-triene,
11-(17-(1,2-Ethylene)-3,6β,16α-trihydroxy-estra-1,3,5(10)-triene-7α-yl)-2-(4,4,5,5,5-pentafluoro-n-pentyl)-undecanoic acid,
11-(17-(1,2-Ethylene)-3,6β,16α-trihydroxy-estra-1,3,5(10)-triene-7α-yl)-2-(4,4,5,5,6,6,7,7,7-nonafluoro-n-heptyl)-undecanoic acid,
17-(1,2-Ethylene)-3,16α-dihydroxy-6-keto-7α-[9-(4,4,5,5,5-pentafluoro-n-pentyl)thiononyl]-estra-1,3,5(10)-triene,
17-(1,2-Ethylene)-3,16α-dihydroxy-6-keto-7α-[9-[(4,4,5,5,5-pentafluoro-n-pentyl)sulfinyl]nonyl]-estra-1,3,5(10)-triene,
17-(1,2-Ethylene)-3,16α-dihydroxy-6-keto-7α-[5-[N-methyl-N-3-(4,4,5,5,5-pentafluoro-n-pentylthio)-propylamino]-pentyl]-estra-1,3,5(10)-triene,
17-(1,2-Ethylene)-3,16α-dihydroxy-6α-methoxy-7α-[9-(4,4,5,5,5-pentafluoro-n-pentyl)thiononyl]-estra-1,3,5(10)-triene,
17-(1,2-Ethylene)-3,16α-dihydroxy-6α-methoxy-7α-[9-[(4,4,5,5,5-pentafluoro-n-pentyl)sulfinyl]nonyl]-estra-1,3,5(10)-triene,
17-(1,2-Ethylene)-3,16α-dihydroxy-6β-methoxy-7α-[9-(4,4,5,5,5-pentafluoro-n-pentyl)thiononyl]-estra-1,3,5(10)-triene, and
17-(1,2-Ethylene)-3,16α-dihydroxy-6β-methoxy-7α-[9-[(4,4,5,5,5-pentafluoro-n-pentyl)sulfinyl]nonyl]-estra-1,3,5(10)-triene.

11. An intermediate compound of the general formula VIII: 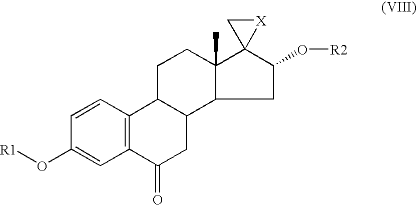
wherein
R1 is H, or a potentially metabolically unstable group chosen from the group consisting of a straight, branched, or cyclic C1-C6 alkyl, C1-C6 acyl, benzoyl. sulphamoyl, and N-acetyl-sulphamoyl;
R2 is H, or a potentially metabolically unstable group chosen from the group comprising C1-C6 acyl or benzoyl; and
X is methylene or a single bond.

12. A compound according to claim 1 for use as a medicament.

13. Use of a compound according to claim 1 for the manufacturing of a medicament for the treatment of an estrogen related disorder or condition that benefits from antiestrogen treatment.

14. Use according to claim 13, wherein the estrogen related disorder or condition is chosen from the group consisting of estrogen dependent breast cancer, anovulatory infertility, menstrual disorders, male pattern baldness, dysfinctional uterine bleeding, endometrial polyps, benign breast disease, uterine leiomyomas, adenomyosis, ovarian cancer, endometrial cancer, melanoma, prostate cancer, cancers of the colon, CNS cancers, endometriosis, polycystic ovary syndrome, infertility and contraception in males.

15. Use according to claim 13 or 14, wherein the estrogen related disorder is estrogen dependent breast cancer.

16. A pharmaceutical composition comprising a compound according to claim 1, admixed with one or more pharmaceutically acceptable excipients or carriers.

17. A pharmaceutical composition according to claim 16, wherein the excipients are chosen from the group comprising filling agents, lubricants, flavours, colourings, sweetenings, buffers, acidifying agents, diluents and preservatives.

18. A pharmaceutical composition according to any one of claims 16-17, for administration orally, intramuscularly, intravenously, intraperitoneally or subcutaneously, via implants, rectally, intranasally, transdermally, or vaginally.

19. A method of treatment comprising administration of a pharmaceutically effective amount of compound according to claim 1 or a pharmaceutical composition according to claim 16 to a subject suffering from an estrogen dependent disorder or condition.

20. A method of treatment according to claim 19, wherein the estrogen dependent disorder or condition is chosen from the group comprising estrogen dependent breast cancer, anovulatory infertility, menstrual disorders, male pattern baldness, dysfimctional uterine bleeding, endometrial polyps, benign breast disease, uterine leiomyomas, adenomyosis, ovarian cancer, endometrial cancer, melanoma, prostate cancer, cancers of the colon, CNS cancers, endometriosis, polycystic ovary syndrome, infertility and contraception in males.

21. A method of treatment according to claim 19, wherein the estrogen dependent disorder is estrogen dependent breast cancer.
